(19) United States
(12) Patent Application Publication
(10) Pub. No.: US 2024/0424175 A1
(43) Pub. Date: Dec. 26, 2024

(54) ANTICOAGULANT COMPOUNDS COMPRISING CHELATING AGENTS AND CATIONIC ANTI-COAGULATION ENHANCERS AND METHODS AND DEVICES FOR THEIR USE

(71) Applicant: Elixir Medical Corporation, Milpitas, CA (US)

(72) Inventors: John YAN, Los Gatos, CA (US); Xiaoxia ZHENG, Fremont, CA (US); Vinayak D. BHAT, Cupertino, CA (US); Motasim SIRHAN, Los Altos, CA (US)

(73) Assignee: Elixir Medical Corporation, Milpitas, CA (US)

(21) Appl. No.: 18/672,288

(22) Filed: May 23, 2024

Related U.S. Application Data
(63) Continuation of application No. PCT/US2022/050099, filed on Nov. 16, 2022.
(60) Provisional application No. 63/282,281, filed on Nov. 23, 2021.
(60) Provisional application No. 63/354,552, filed on Jun. 22, 2022.

(51) Int. Cl. A61L 31/16; A61L 31/08

(52) U.S. Cl. CPC A61L 31/16; A61L 31/08; A61L 2300/208; A61L 2300/214; A61L 2300/42; A61L 2300/434; A61L 2300/45; A61L 2300/602; A61L 2300/606

(57) ABSTRACT

Devices, systems, and methods are provided including a structure having one or more surfaces configured for internal use within a patient's body and one or more therapeutic compositions comprising one or more active substances including at least one of a chelating agent, a direct factor Xa inhibitor, a direct factor IIa inhibitor, and a factor XI/XIa inhibitor disposed in or on the structure. The structure is configured to be positioned adjacent a target site in the patient's body. The therapeutic composition is formulated to release the one or more active substances to the target site to provide one or more of inhibit clot formation, promote clot dissolution, inhibit or dissolute inflammation, inhibit vessel injury, increase time before clotting, and/or inhibit cell proliferation. Delayed or other controlled release of the therapeutic composition from the structure may be provided.

### CROSS-REFERENCE TO RELATED APPLICATIONS

This application is a continuation of PCT Application No. PCT/US2022/050099 (Attorney Docket No. 32016-729.601), filed Nov. 16, 2022, which claims the benefit of U.S. Provisional Application 63/282,281 (Attorney Docket No. 32016-729.101), filed on Nov. 23, 2021, and of U.S. Provisional Application 63/354,552 (Attorney Docket No. 32016-729.102), filed on Jun. 22, 2022, the full disclosures of which are incorporated herein by reference.

### BACKGROUND

### Field of the Disclosure

The present disclosure relates generally to medical devices and methods and more particularly to the coating of implantable and other devices with anticoagulant compositions.

Blood coagulation is a process designed to stop bleeding from a damaged blood vessel. This process requires coagulation factors, calcium and phospholipids. It is initiated by extrinsic tenase, which forms when factor VIIa binds to tissue factor. Extrinsic tenase activates factors IX and X. In the presence of calcium, factor IXa binds to negatively charged phospholipid surfaces where it interacts with factor VIIIa to form intrinsic tenase, a complex that efficiently activates factor X. Factor Xa binds to factor Va on negatively charged phospholipid surfaces to form prothrombinase, the complex that activates prothrombin (factor II) to thrombin (factor IIa). Thrombin then converts fibrinogen to fibrin. Activated platelets or monocytes provide negatively charged phospholipid surfaces on which these clotting reactions occur. The intrinsic pathway is initiated by negatively charged surfaces-mediated activation of factor XII (FXII). Such contact activation further propagates thrombin generation by sequential activation of FXI, FIX, FX, and prothrombin. Importantly, thrombin can further activate FXI in a feedback mechanism. Thrombin also activates platelets, which can subsequently support FXI activation. Activation of FXI leads to enhanced thrombin formation, thus forming a positive feedback loop for thrombin formation and consolidation of coagulation. Disorders of coagulation can lead to obstructive clotting (thrombosis) or occlusion of the blood vessel.

Damage to a blood vessel can be caused by, e.g., injurious contact of a device employed in a surgery or intervention with the blood vessel (e.g., a surgical knife cutting a tissue containing the blood vessel, or a deployed stent embedding into the wall of the blood vessel). Damage to the blood vessel can lead to abnormal or undesired recruitment, activation, and/or proliferation of proteins (e.g., fibrin) and cells (e.g., platelets) involved in the coagulation process and other processes at the site of injury, which can result in obstructive clotting or occlusion of the blood vessel.

After vascular injury von Willebrand factor acts as a bridge between endothelial collagen and platelet surface receptors GpIb and promotes platelet adhesion. After adhesion, degranulation from both types of granules takes place with the release of various factors. Release of calcium occurs here simultaneously. Calcium binds to the phospholipids that appear secondary to the platelet activation and provides a surface for assembly of various coagulation factors.

Calcium ions play an important role in the tight regulation of coagulation cascade that is paramount in the maintenance of hemostasis. Calcium ions are essential in coagulation cascade as it is a cofactor for membrane-bound complexes, including intrinsic tenase (FIXa-FVIIIa), extrinsic tenase (FVIIa-TF) and prothrombinase (FXa-FVa) complex.

Under normal physiological conditions, normal vascular endothelium minimizes contact between tissue factor (TF) and plasma procoagulants, but vascular insult expose tissue factor which binds with factor VIIa and calcium to promote the conversion of factor X to Xa.

Other than platelet activation, calcium ions are responsible for complete activation of several coagulation factors, including coagulation Factor XIII (FXIII). FXIII is responsible for covalently cross-linking preformed fibrin clots preventing their premature fibrinolysis, by maintaining the clot architecture and strength. The time lag in generation of first FXIIIa molecule is about 10 minutes to 20 minutes. To prevent the crosslinking of premature fibrin clot, this 10 minutes to 20 minutes is highly critical.

Anticoagulants can be used to prevent the formation of blood clots. Some are used for the prevention or treatment of disorders characterized by abnormal blood clots and emboli. By reducing blood clotting, anticoagulants can prevent deep vein thrombosis, pulmonary embolism, myocardial infarction, and ischemic stroke.

Blood coagulation process requires coagulation factors, calcium and phospholipids. There are three strategies to prevent coagulation. (1) inhibits the clotting factors. (2) removal of free calcium in the blood. (3) change of negatively charged phospholipid surfaces as neutral or positive charged such that the clotting reactions cannot occur because these clotting factors can only bind to a negatively charged phospholipid surface which is provided by activated platelets or monocytes. Some anticoagulant drugs act by inactivating thrombin and several other clotting factors that are required for a clot to form. There are other anticoagulant drugs act by removal free calcium ions or inhibit phospholipid such as platelet activating factor. Removal of free calcium ions can be accomplished by the chelating agent EDTA, citate or oxalate. EDTA inhibits the clotting factors intrinsic XII, XI, IX, X and extrinsic VIIa/TF activation and inhibit initial the clotting cycles by depleting/chelating free calcium ions. This is critical for extrinsic pathway, which is the only step (VIIa/TF activated by Ca2+) involved in the cascade of coagulation. The mechanism of EDTA as an agent to prevent clotting induced by a medicated stent is that its inhibition of adenosine, epinephrine, and thrombin-induced platelet aggregation might be more effective than mechanisms that inhibit platelet aggregation more narrowly, as is the case of clopidogrel, which inhibits only adenosine-induced aggregation.

FXIa inhibitor is an active-site inhibitor, and it achieves antithrombotic activity without increasing bleeding risk. It also delays the time to clot formation, decreases fibrin incorporation into the clot, and reduces the resistance of clots to fibrinolysis.

Systemic administration of an anticoagulant may be ineffective in preventing or treating disorders associated with coagulation. For example, the concentration of the anticoagulant at or adjacent the site of injury may be insufficient at the appropriate time to prevent or treat disorders associated with coagulation. Furthermore, deficiencies of systemic administration of an anticoagulant can be exacerbated where the patient has a condition (e.g., cardiovascular disease, hypercholesterolemia, or diabetes) that renders the patient more susceptible to a vaso-occlusive event.

Previous attempts to provide local administration of an anticoagulant have had limited to no success in preventing coagulation disorders and/or preventing thrombus (clot) formation particularly after local tissue injury. Furthermore, local injury to a tissue is commonly associated with additional injury to the tissue adjacent (e.g., proximal, distal, etc.) the site of the first injury.

To reduce the partial or total occlusion of the artery by plaque or the collapse of the arterial lining and to reduce the chance of restenosis, a stent or drug coated balloon may be used in the artery to keep the artery open. The agents coated on stent or balloon unfortunately may delay the healing period of the injured tissue, increase tissue factor which may generate or amplify thrombin, fibrin, and/or clot formation, especially within the first 3 hours to 72 hours or more: however, the time lag to generate the first molecule FXIIIa, which is responsible for covalently cross-linking pre-matured fibrin clots and activated by calcium ions is about 10 minutes to 20 minutes. To prevent the crosslinking of premature fibrin clot, this 10 minutes to 20 minutes is highly critical.

Anticoagulants can be used to prevent the formation of blood clots. Some are used for the prevention or treatment of disorders characterized by abnormal blood clots and emboli. By reducing blood clotting, anticoagulants can prevent deep vein thrombosis, pulmonary embolism, myocardial infarction, and ischemic stroke.

The purpose of adding implanted medication to a stent is to prevent thrombin accumulation and restenosis. However, due to an increase in thrombosis at the site of the stent, the risk of death and the risk of myocardial infarction (MI) increased. Although long term treatment with clopidogrel bisulfate plus aspirin for at least 12 months has been suggested as a preventive treatment, there is no evidence that this treatment is effective for more than six months. Clopidogrel also increases the risk of major bleeding episodes.

There is still a need to develop specialized therapeutic compositions for medical devices that can rapidly deliver therapeutic agents, drugs, or bioactive materials directly into a localized tissue area during or following a medical procedure, so as to treat or prevent vascular and nonvascular diseases or conditions such as restenosis or thrombosis. The device should release the therapeutic agent in an effective and efficient manner at the desired target location, where the therapeutic agent should rapidly permeate the target tissue at a local therapeutic level to inhibit one or more of thrombin, fibrin, and/or clot formation prior to amplification of the clotting factors.

It would therefore be desirable to provide devices that locally deliver thrombin/clot formation-inhibiting agents, and optionally other kinds of biologically active agents (e.g., anti-proliferative agents, anti-inflammatory agents, etc.), to the site of injury of a body part or to an area adjacent thereto before, during, and/or after injury. The disclosure also provides methods of using such devices and other forms of therapy in treating clotting, and in improving or promoting wound healing, at the injury site or at an area adjacent thereto.

### Listing of Background Art

Relevant background art includes U.S. Pat. Nos. 9,770,349; 9,901,663; 6,500,855; 8,409,272; 8,946,219; US2003/0158120; US2005004663; US2005/0064006; US2009/0075949; US2010/0003542; US2010/0130543; US2010/0184729; US2010/049328; US2013/189329; US2015/157771; US2018/0000490; EP 1849434; CA2464290; WO2013/007840; WO2013/056060; WO2020/210613; and WO2020/210629.

The subject matter of this application is also related to that of the following commonly owned applications: International Patent Application PCT/US2021/034108 filed May 25, 2021, entitled “Anticoagulant compositions and methods and devices for their use” (Attorney Docket No. 32016-720.601): International Patent Application PCT/US2021/044414 filed Aug. 3, 2021, entitled “Anticoagulant compositions and methods and devices for their pulmonary use” (Attorney Docket No. 32016-730.601); International Patent Application PCT/US2021/049964 filed Sep. 10, 2021, entitled “Anticoagulant compositions and methods and devices for their ophthalmic use” (Attorney Docket No. 32016-720.601): International Patent Application No. PCT/US2007/078317, filed Sep. 12, 2007, entitled “Macrocyclic lactone compounds and methods for their use” (Attorney Docket No. 32016-704.601): International Patent Application No. PCT/US2008/056501, filed Mar. 11, 2008, entitled “Macrocyclic lactone compounds and methods for their use” (Attorney Docket No. 32016-704.602): International Patent Application No. PCT/US2009/059396, filed Oct. 2, 2009, entitled “Macrocyclic lactone compounds and methods for their use” (Attorney Docket No. 32016-709.601); International Patent Application No. PCT/US2011/054637, filed Oct. 3, 2011, entitled “Macrocyclic lactone compounds and methods for their use” (Attorney Docket No. 32016-711.601); each of which are incorporated herein by reference for all purposes in their entireties.

### SUMMARY OF THE INVENTION

In a first aspect, the present invention provides an implantable scaffold comprising a scaffold structure having a surface configured to be expanded in a patient's body. A therapeutic composition, comprising at least one chelating agent is present on a surface of the scaffold, where the therapeutic composition is formulated for a rapid release of the chelating agent into an environment surrounding the scaffold upon implantation of the scaffold structure in said environment.

In some examples, the scaffold structure may be configured to be expanded in a vascular lumen or any other target site in the patient's body. The therapeutic composition may be present at least partly on the surface of scaffold structure. Alternatively, the therapeutic composition is present at least partly within a cavity or reservoir within the scaffold structure.

Exemplary chelating agents in the therapeutic composition may be formulated to deplete calcium in the environment surrounding the scaffold upon implantation of the scaffold structure in said environment.

In specific instances, the therapeutic composition may be formulated to release at least 50%, preferably at least 75%, by weight of the at least one chelating agent into the vascular environment within 72 hours of implantation, preferably within 24 hours of implantation, more preferably within 6 hours of implantation, and even more preferably within 4 hour of implantation.

In additional instances, the therapeutic composition may be formulated to release additional amounts of the at least one chelating agent into the environment for a period of at least 3 days, preferably at least 7 days, more preferably 21 days, still more preferably at least 28 days, even more preferably at least 3 months, and often 6 months or more after implantation.

Exemplary chelating agents may be selected from a group consisting of ethylenediaminetetraacetic acid (EDTA), calcium disodium edetate, magnesium dipotassium edetate, and magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA). 1,2-bis(2-aminophenoxy) ethane-N,N,N′,N′-tetraacetic acid (BAPTA), deferoxamine (DFO), a surfactant-EDTA complex, quaternary ammonium EDTA salt, benzalkonium EDTA salt. EDTA complex, salts, analogue, solvate, hydrate and derivatives thereof.

In particular instances, the chelating agent consists essentially of ethylenediaminetetraacetic acid (EDTA).

The therapeutic composition that may comprise, consist essentially of, or consist of chelating agent, where the chelating agent may be present in the therapeutic composition at a weight percent from 10% to 100%. In specific instances, the therapeutic composition consists essentially of chelating agent, where the only active ingredient in the therapy composition will be the chelating agent optionally present with other inactive components and ingredients. In other instances, the therapeutic composition comprises the chelating agent in combination with additional active and/or inactive substances. In such instances, the additional active and/or inactive substances may be present in the therapeutic composition at a weight percent from 20% to 90%.

In some instances, the therapeutic compositions of the present invention may further comprise a cationic anti-coagulation enhancer, where the cationic anti-coagulation enhancer may selected from a group consisting of magnesium stearate and other magnesium salts, monoammonium salts, diammonium salts, triammonium salts, benzyldimethyltetradecylammonium salts, tridodecylmethylammonium salt, other benzalkonium, analogue, solvate, hydrate and derivatives thereof. Alternatively or additionally, the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan. PAMAM dendrimers, and poly(2-dimethylamino)ethyl methacrylate (pDMAEMA), protamine, polylysine, a poly betaaminoester (PBAE). Histone, ethylenediamine, methylenediamine, ammonium chloride, melamine, histamine, histidine, analogue, solvate, hydrate and derivatives thereof.

In specific examples, the cationic anti-coagulation enhancer may comprise, consist essentially of, or consist of benzyldimethyltetradecylammonium chloride.

In specific examples, the cationic anti-coagulation enhancer may comprise, consist essentially of, or consist of linear polyethyleneimine (PEI).

Any of the above described therapeutic compositions may further comprise at least one anti-coagulant. In such instances, the therapeutic composition may be formulated to release at least one anti-coagulant at a rate equal to that of the chelating agent. Alternatively, the therapeutic composition may be formulated to release at least one anti-coagulant at a rate slower than that of the chelating agent. Alternatively, the therapeutic composition may be formulated to release at least one anti-coagulant at a rate faster than that of the chelating agent.

In specific examples, the anti-coagulant may be selected from the group consisting of a direct factor IIa inhibitor and a direct factor Xa inhibitor. Exemplary direct factor IIa inhibitors may be selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. A preferred, direct factor IIa inhibitor comprises argatroban, or a salt, isomer, solvate, analog, derivative, metabolite, or prodrugs thereof.

Exemplary direct factor Xa inhibitors may be selected from the group consisting of apixaban, betrixaban, edoxaban, otamixaban, razaxaban, rivaroxaban. (r)-n-(2-(4-(1-methylpiperidin-4-yl) piperazin-1-v1)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribavaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). A first preferred direct factor Xa inhibitor comprises apixaban, or a salt, isomer, solvate, analog, derivative, metabolite, or prodrugs thereof. A second preferred direct factor Xa inhibitor comprises rivaroxaban, or a salt, isomer, solvate, analog, derivative, metabolite, or prodrugs thereof.

Any of the above described therapeutic compositions may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. A preferred MTOR inhibitor comprises sirolimus, or a salt, isomer, solvate, analog, derivative, metabolite, or prodrugs thereof.

Any of the above described therapeutic compositions may further comprise paclitaxel, or a salt, isomer, solvate, analog, derivative, metabolite, or prodrug thereof.

Any of the above described therapeutic compositions may further comprise an antiplatelet drug. Any of the above described therapeutic compositions may further comprise an antiproliferative agent selected from the group consisting of mycophenolate mofetil, mycophenolate sodium, azathioprine.

The implantable scaffolds of present invention may have anyone of a wide variety of known structures suitable for implantation and expansion at a target site in the patient's body. Often, the scaffold will have at least an outer surface, an inner surface, and one or more edge surfaces between the outer and inner surfaces. In such instances, at least a portion of the outer surface may be coated with the therapeutic compositions. In other instances, at least a portion of the inner surface may be coated with the therapeutic compositions. In still other instances, a portion of the edge surfaces may be coated with the therapeutic compositions. As an alternative or in addition to surface coating, at least some of the surfaces may have receptacles formed therein and at least some of said receptacles have therapeutic agent therein. For example, the receptacles comprise one or more of wells, channels, holes, and surface texture.

In a second aspect, the present invention provides a method for treating a vascular tissue injury in a patient. The method comprises implanting a scaffold structure at a target location in the patient's vasculature proximate the tissue injury and releasing a drug composition including at least one chelating agent from the implanted scaffold structure into the vasculature, wherein the chelating agent is released sufficiently rapidly into the vasculature to prevent blood clotting and inhibit the fibrin formation.

In particular examples, at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In the further examples of the methods herein, the chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA). 1,2-bis(2-aminophenoxy) ethane-N,N,N′,N′-tetraacetic acid (BAPTA), deferoxamine (DFO), a surfactant-EDTA complex, quaternary ammonium EDTA salt, benzalkonium EDTA salt. EDTA complex, salts, analogue, solvate, hydrate and derivative thereof. In one specific instance, the chelating agent consists essentially of ethylenediaminetetraacetic acid (EDTA).

In some instances, therapeutic composition further comprises a cationic anti-coagulation enhancer. For example, the cationic anti-coagulation enhancer may be selected from a group consisting of magnesium stearate and other magnesium salts, monoammonium salts, diammonium salt, triammonium salt, benzyldimethyltetradecylammonium salt, tridodecylmethylammonium salt, other benzalkonium, analogue, solvate, hydrate and derivatives thereof. In another example,
the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a polybetaaminoester (PBAE). Histone, ethylenediamine, methylenediamine, ammonium chloride, melamine, histamine, histidine, analogue, solvate, hydrate and derivatives thereof. In a specific instance, the cationic anti-coagulation enhancer consists essentially of benzyldimethyltetradecylammonium chloride. In another specific instance, the cationic anti-coagulation enhancer consists essentially of linear polyethyleneimine (PEI).

The therapeutic composition delivered by the methods of the present invention may further comprise at least one anti-coagulant. For example, the at least one anti-coagulant may be selected from the group consisting of a direct factor IIa inhibitor and a direct factor Xa inhibitor. Alternatively, the at least one anti-coagulant may comprise a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. An exemplary direct factor IIa inhibitor may comprises argatroban, or a salt, isomer, solvate, analog, derivative, metabolite, or prodrugs thereof.

In other examples of the methods herein, the at least one anti-coagulant may comprise a direct factor Xa inhibitor selected from the group consisting of apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In one instance, the direct factor Xa inhibitor may comprise apixaban, or a salt, isomer, solvate, analog, derivative, metabolite, or prodrugs thereof. In another instance, the direct factor Xa inhibitor may comprise rivaroxaban, or a salt, isomer, solvate, analog, derivative, metabolite, or prodrugs thereof.

The therapeutic composition delivered by the methods of the present invention may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. An exemplary mTOR inhibitor comprises sirolimus, or a salt, isomer, solvate, analog, derivative, metabolite, or prodrugs thereof.

The therapeutic composition delivered by the methods of the present invention may further comprise paclitaxel, or a salt, isomer, solvate, analog, derivative, metabolite, or prodrug thereof.

The therapeutic composition delivered by the methods of the present invention may further comprise further comprises an antiplatelet drug.

In all of the methods described herein, the therapeutic composition may be positioned on at least one of an internal surface and an external surface of the implantable scaffold and/or may be positioned on both an external and an internal surface of the implantable scaffold.

The methods of the present invention are generally suitable for treating tissue injury caused by expanding the scaffold at the location but are also useful for treating tissue injury that preexists deploying the structure at the location.

In a third aspect, the present invention provides an implant comprising a body structure having a surface configured to be implanted in a patient's body. A therapeutic composition is present on a surface of the body structure, where the therapeutic composition comprises at least one drug selected from the group consisting of a chelating agent, a direct factor IIa inhibitor, an a direct factor Xa inhibitor, wherein the therapeutic composition is formulated for a delayed release into an environment surrounding the body structure upon implantation of the body structure into said environment.

In some examples, the therapeutic composition may be formulated for a rapid release into the environment surrounding the body structure a preselected time period after implantation of the body structure into said environment.

Implants may be any type of therapeutic, diagnostic, or other structure intended for implantation in the patient's body, typically being an expandable scaffold, such as a vascular stent, a prosthetic heart valve, a patent foramen ovale (PFO) occlusion device, an atrial septal defect (ASD) occlusion device, a left atrial appendage (LAA) occlusion device, or similar expandable structure, or being an orthopedic implant.

In such instances, the therapeutic composition may be present at least partly on the surface of the body structure. Alternatively or additionally, the therapeutic composition may be present at least partly within a cavity or reservoir within the body structure.

The therapeutic composition may be formulated to inhibit release of the at least one drug into the environment surrounding the body structure for a time period in a range having a lower time limit selected from 5 minutes, 10 minutes, 15 minutes, 30 minutes, and 45 minutes and an upper time limit selected from 1 hour, 2 hours, 3 hours, and 4 hours, and all combinations thereof.

The therapeutic composition may be formulated to release at least 50% by weight, preferably at least 75% by weight of the at least one drug into the environment surrounding the body structure within 72 hours of implantation, preferably within 24 hours of implantation, more preferably within 6 hours of implantation, and even more preferably within 4 hour of implantation.

The therapeutic composition may be formulated to release additional amounts of the at least one drug into the environment for a period of at least 3 days, preferably at least 7 days, more preferably 21 days, still more preferably at least 28 days, even more preferably at least 3 months, and often 6 months or more after implantation.

In some examples, the drug in the therapeutic composition may comprise at least a chelating agent in the therapeutic composition is formulated to deplete calcium in the environment surrounding the body structure upon implantation of the body structure in said environment. Such a chelating agent may be selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy) ethane-N,N,N′,N′-tetraacetic acid (BAPTA), deferoxamine (DFO), a surfactant-EDTA complex, quaternary ammonium EDTA salt, benzalkonium EDTA salt. EDTA complex, salts, analogue, solvate, hydrate and derivatives thereof. A specific instance, the chelating agent consists essentially of ethylenediaminetetraacetic acid (EDTA).

In some instances, the therapeutic composition may further comprise a cationic anti-coagulation enhancer. For example, the cationic anti-coagulation enhancer may be selected from a group consisting of magnesium stearate and other magnesium salts, monoammonium salts, diammonium salts, triammonium salts, benzyldimethyltetradecylammonium salts, tridodecylmethylammonium salts, other benzalkonium, analogue, solvate, hydrate and derivatives thereof. Alternatively or additionally, the cationic anti-coagulation enhancer may be selected from a group consisting of cationic polymer or compounds including but not limit to poly(L-lysine) (PLL), linear polyethyleneimine (PEI), branch polyethyleneimine (PEI), chitosan. PAMAM dendrimers, and poly(2-dimethylamino)ethyl methacrylate (pDMAEMA), protamine, polylysine, a polybetaaminoester (PBAE). Histone, ethylenediamine, methylenediamine, ammonium chloride, melamine, histamine, histidine, analogue, solvate, hydrate and derivatives thereof. In a specific instance, the cationic anti-coagulation enhancer consists essentially of benzyldimethyltetradecylammonium chloride. In another specific instance, the cationic anti-coagulation enhancer consists essentially of linear polyethyleneimine (PEI).

In some instances, the at least one drug may comprise a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin. For example, the at least one direct factor IIa inhibitor may comprise argatroban, or a salt, isomer, solvate, analog, derivative, metabolite, or prodrugs thereof.

In some examples, the at least one drug may comprise a direct factor Xa inhibitor selected from the group consisting of apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). For example, the direct factor Xa inhibitor may comprise apixaban, or a salt, isomer, solvate, analog, derivative, metabolite, or prodrugs thereof. In another example, the direct factor Xa inhibitor may comprise rivaroxaban, or a salt, isomer, solvate, analog, derivative, metabolite, or prodrugs thereof.

In some examples, the at least one drug may comprise a factor XI/XIa inhibitor selected from the group consisting of protein Z-dependent protease inhibitors (ZPI).

In specific examples, a ZPI-containing cationic anti-coagulation enhancer may comprise, consist essentially of, or consist of milvexian, a small molecule factor XIa inhibitor having the formula [(6r,10s)-10-{4-[5-chloro-2-(4-chloro-1h-1,2,3-triazol-1-yl)phenyl]-6-oxo-1 (6h)-pyrimidinyl}-1-(difluoromethyl)-6-methyl-1,4,7,8,9,10-hexahydro-11,15-(metheno) pyrazolo [4,3-b][1,7] diazacyclotetradecin-5 (6h)-one] and described in WO2020/210613 and WO2020/210629, the full disclosures of which are incorporated herein by reference.

In other examples, therapeutic composition may further comprise an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. The particular instance, the mTOR inhibitor comprises sirolimus, or a salt, isomer, solvate, analog, derivative, metabolite, or prodrugs thereof.

In still other examples, the composition may further comprise paclitaxel, or a salt, isomer, solvate, analog, derivative, metabolite, or prodrug thereof.

In still other examples, the therapeutic composition may further comprise an antiplatelet drug.

In still other examples, the therapeutic composition may further comprise an antiproliferative agent selected from the group consisting of mycophenolate mofetil, mycophenolate sodium, azathioprine.

The delayed composition release implantable scaffolds may have at least an outer surface, an inner surface, and one or more edge surfaces between the outer and inner surfaces, where at least a portion of the outer surface may be coated with the therapeutic compositions. Additionally or alternatively, at least a portion of the inner surface may be coated with the therapeutic compositions. Additionally or alternatively, at least a portion of the edge surfaces may be coated with the therapeutic compositions. Additionally or alternatively, at least some of the surfaces may have receptacles formed therein and at least some of said receptacles have therapeutic agent therein. For example, the receptacles comprise one or more of wells, channels, holes, and surface texture.

In another aspect, the present invention provides an implantable scaffold comprising a scaffold structure having a surface configured to be expanded in the patient's body. A first therapeutic composition is coated, layered, bonded, or otherwise affixed to the scaffold and comprises a first drug formulation including at least one drug selected from the group consisting of a calcium chelating agent, a direct factor IIa inhibitor and a direct factor Xa inhibitor. A second therapeutic composition is also coated, layered, bonded, or otherwise affixed to the scaffold structure and or the first therapeutic composition and comprises a second drug formulation including at least one drug selected from the group consisting of a calcium chelating agent, a direct factor IIa inhibitor and a direct factor Xa inhibitor. The first therapeutic composition is formulated for a rapid release of the first drug formulation into a vascular environment and the second therapeutic composition is formulated for an extended release of the second drug formulation into the vascular environment.

The implantable scaffold may have any conventional or novel structure intended for implantation in a patient's vasculature, including, the arterial and venous coronary, peripheral and cerebral vasculature. The scaffolds may be intended for direct implantation, for example comprising or consisting of vascular stents intended to maintain patency in in a vascular lumen. Additionally, the scaffolds may be part of assemblies including additional components, such as vascular grafts, prosthetic valves, and the like. Depending on the intended purpose, the scaffold may be non-degradable in the vascular environment, for example being formed from or otherwise comprising a metal or a polymer which is non-degradable in the vascular environment. In other instances, the scaffold may be degradable in the vascular environment, for example being formed from or otherwise comprising a metal or polymer which is degradable in the vascular environment.

In addition to such implantable scaffolds, the therapeutic compositions and drug formulations described below may also find use with a wide variety of other implantable and non-implantable devices and tools which may be subject to unwanted clotting, as described elsewhere herein.

The rapid release of the first drug formulation and extended release of the second drug formulation will typically act in combination to accelerate dissolution of one or more of inflammation, cell proliferation, internal elastic lamina (IEL) injury, thrombin, fibrin formation, platelet aggregation, platelet activation, and clot or thrombus formation; and/or inhibit one or more of inflammation, cell proliferation, internal elastic lamina (IEL) injury, thrombin, fibrin formation, platelet aggregation, platelet activation, and clot or thrombus formation; and/or increase or prolong time before blood forms clot or thrombus.

In specific instances, at least one of the first drug formulation, the second drug formulation, and the third drug formulation may comprise a calcium chelating agent, a direct factor IIa inhibitor and a direct factor Xa inhibitor. In other instances, the first drug formulation, the second drug formulation, and the third drug formulation may each comprise a calcium chelating agent, a direct factor IIa inhibitor, and a direct factor Xa inhibitor.

In specific instances, the at least one drug of the first (rapid release) drug formulation is released from the first therapeutic composition over a first time period (duration) is in a range from 5 minutes to 28 days after implantation, usually from 5 minutes to 3 days after implantation, and preferably from 5 minutes to 1 days after implantation. The first therapeutic composition is typically configured to release the at least one drug of the first drug formulation at a mean rate in the range from 2 μg/hour to 40 μg/hour, usually from 2 μg/hour to 30 μg/hour, preferably from 2 μg/hour to 10 μg/hour over a 24-hour period following exposure to the vascular environment, where the mean rate may be determined based on the amount (weight) of drug released over the total duration of the release.

In specific instances, the at least one drug of the second drug formulation (sustained release) is released from the second therapeutic composition over a second time period is in a range from 30 days to 12 months after implantation, usually from 30 days to 9 months after implantation, and preferably from 30 days to 6 months after implantation. The second therapeutic composition is typically configured to delay release the at least one drug of the second drug formulation for at least one 24-hour period following exposure to the vascular environment. The second therapeutic composition is typically configured to release the at least one drug of the second drug formulation at a mean rate not exceeding 2 μg/hour, usually 1 μg/hour, preferably 0.5 μg/hour, and more preferably 0.1 μg/hour after the 24 hour period following exposure to the vascular environment, where the mean rate may be determined based on the amount (weight) of drug released over the total duration of the release.

The first and second therapeutic composition will typically but not necessarily comprise a carrier, matrix or coating, usually but not always including a polymer, to sequester and control the release rate and duration of the drugs. In some instances, the drugs may be coated, layered, or otherwise deposited on or in surfaces or receptacles on the implantable structure without a polymer or other carrier but optionally with excipients, coating agents, and other conventional drug coating materials.

In some instances, one of the first and second therapeutic compositions may comprise a polymer while the other is free from polymer. For example, the first therapeutic (rapid release) composition may free from polymer and the second (sustained release) therapeutic composition may comprises a polymer to maintain or control the release rate and duration. For example, the first therapeutic composition may be coated on the scaffold structure or over the second therapeutic composition to affect a burst release.

In instances where the first and second therapeutic compositions each comprise a polymer, the first therapeutic composition will have a first drug-to-polymer weight ratio and the second therapeutic composition will a second drug-to-polymer weight ratio. The ratios may be the same but will more often be different. For example, the first drug-to-polymer weight ratio may be in a range from 5:1 to 1:3, usually from 5:2 to 1:2, and preferably from 5:3 to 1:1, and the second drug-to-polymer weight ratio may in a range from 5:2 to 1:5, usually from 5:3 to 2:5, and preferably from 1:1 to 1:2. The first drug-to-polymer weight ratio is usually greater than the second drug-to-polymer weight ratio (greater loading can enhance the burst effect in the first therapeutic composition), but in some instances the first drug-to-polymer weight ratio may less than the second drug-to-polymer weight ratio (greater loading can also enhance duration of release).

While drug release from the first and second therapeutic compositions may commence simultaneously, in many instances the first therapeutic composition and the second therapeutic composition are configured to delay start of release of the second drug formulation for a time period after release of the first drug formulation has started. For example, the first therapeutic composition may be layered over the second therapeutic composition to delay release of the second drug formulation, e.g, the first therapeutic composition may initially cover at least a portion of the second therapeutic composition and may be configured to dissolve over the time period in the vascular environment to expose the second therapeutic composition and allow release of the second drug formulation.

Alternatively, a sacrificial layer may present over at least one of the first therapeutic composition and the second therapeutic composition or between the first therapeutic composition and the second therapeutic composition to delay release of one or more drugs from either or both of the first therapeutic composition and the second therapeutic compositions.

Alternatively, a diffusion-rate controlling layer may be present over at least one of the first therapeutic composition and the second therapeutic composition or between the first therapeutic composition and the second therapeutic composition to control a release rate of one or more drugs from either or both of the first therapeutic composition and the second therapeutic compositions.

The polymer(s) may be configured to release the first and/or second drug formulation at least partly by dissolution of the polymer when exposed to the vascular environment. For example, the polymer of the first therapeutic composition may dissolve at a faster rate than dissolution of the second therapeutic composition in the vascular environment. Alternatively, the polymer may be configured to release the first and/or second drug formulation at least partly by a diffusion mechanism through the polymer when exposed to the vascular environment. Alternatively, the polymer may be configured to release the first and/or second drug formulation through a combination of dissolution of and diffusion through the polymer when exposed to the vascular environment.

Usually, but not necessarily, one or more polymer will be porous where the first and/or second drug formulation are sequestered in pores of the polymer(s). Often, a release rate of the first and/or second drug formulation may at least partly be determined by a pore size of the polymer. In some instances, the polymers of the first and second drug formulations may have different pore sizes which provide different release rates.

In other instances, the first and second drug formulations may be at least partially separated in different regions within the porous polymer. Alternatively or additionally, the first and second drug formulations may at least partially present in overlapping regions of the porous polymer.

In preferred instances, the implantable scaffold of the present invention will further comprise an anti-proliferative drug. The anti-proliferative drug is present in either or both of the first and second drug formulations or may be present in a third drug formulation or may be separately coated, coupled, bonded or attached to the scaffold. For example, the anti-proliferative drug may present in a third therapeutic composition formulated to release the anti-proliferative drug into a vascular environment when the scaffold is present in the vascular environment.

The first, second, and optionally third or additional therapeutic compositions of the present invention may be positioned on an external, internal, edge, and/or other surface of the implantable scaffold. Optionally but not necessarily, the scaffold surfaces will be roughened, scored, etched, or otherwise treated to enhance attachment of the therapeutic compositions. In some instances, the therapeutic compositions may be sequestered in wells, indentations or other receptacles formed on or in the scaffold surfaces.

Exemplary calcium chelating agents of the present invention is selected from a group consisting of ethylenediaminetetraacetic acid (EDTA), calcium disodium edetate to calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA). 1,2-bis(2-aminophenoxy) ethane-N,N,N′,N′-tetraacetic acid (BAPTA), citrate, oxalate, the surfactant-EDTA complex, quaternary ammonium EDTA salt, benzalkonium EDTA salt, EDTA complex, salts, analogue, solvate, hydrate and derivative thereof.

Exemplary of EDTA complex the present invention include monoammonium EDTA salt, diammonium EDTA salt, triammonium EDTA salt, benzyldimethyltetradecylammonium EDTA salt, tridodecylmethylammonium EDTA salt, other benzalkonium EDTA salt, and any chemical could complex with EDTA.

Exemplary direct factor IIa inhibitors of the present invention include argatroban, dabigatran, ximelagatran, melagatran, efegatran, inogatran, atecegatran metoxil (AZD-0837), hirudin, hirudin analogs, bivalirudin, desirudin, and lepirudin, which may be used individually or in combination. Preferred direct factor IIa inhibitors comprise argatroban, or a salt, isomer, solvate, analog, derivative, metabolite, or prodrugs thereof.

Exemplary direct factor Xa inhibitors of the present invention include apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), which may be used individually or in combination. Preferred direct factor Xa inhibitor comprise (1) apixaban, or a salt, isomer, solvate, analog, derivative, metabolite, or prodrugs thereof and (2) rivaroxaban, or a salt, isomer, solvate, analog, derivative, metabolite, or prodrugs thereof.

Exemplary anti-proliferative agents of the present invention include mycophenolate mofetil, mycophenolate sodium, azathioprine, mTOR inhibitors selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof, which may be used individually or in combination. Preferred anti-mTOR proliferative agents comprise sirolimus, or a salt, isomer, solvate, analog, derivative, metabolite, or prodrugs thereof.

Exemplary anti-proliferative agents of the present invention also include paclitaxel, or a salt, isomer, solvate, analog, derivative, metabolite, or prodrug thereof.

In addition to the first, second, and optional third therapeutic compositions as discuss above, the implantable scaffolds of the present invention may further comprise at least one additional drug, typically an antiplatelet drug. The additional drug will not necessarily be incorporated as a drug formulation or as part of a therapeutic composition.

In specific examples of the present invention, the direct factor IIa inhibitor comprises argatroban and the direct factor Xa inhibitor comprises apixaban or rivaroxaban. In other specific examples of the present invention, the direct factor IIa inhibitor comprises argatroban or an analogue of argatroban, the direct factor Xa inhibitor comprises apixaban or rivaroxaban or an analogue of apixaban or rivaroxaban, and the anti-proliferative agent comprises sirolimus or an analogue of sirolimus.

In some instances, at least one of the therapeutic compositions may comprises an excipient, an adjuvant, a carrier, a wetting agent. In some instances, the first and second therapeutic compositions may be formed contiguously. In some instances, the first and second therapeutic compositions are separated by barrier, for example a polymer layer.

In some examples, a third therapeutic composition comprises a third drug formulation including at least one drug selected from the group consisting of a calcium chelating agent, a direct factor IIa inhibitor and a direct factor Xa inhibitor. The third drug formulation may comprise any one of the previously discussed drugs and/or an additional drug. The third therapeutic composition may be disposed at least partially over the first therapeutic composition which may disposed at least partially over the second therapeutic composition, where the third therapeutic composition may be configured to effect a burst release which is more rapid than the release of either the first or second therapeutic compositions.

In specific examples, the first and second therapeutic compositions may comprise polymer and the third therapeutic composition may be free from polymer and coated or otherwise deposited over at least a portion of the first therapeutic composition.

In other examples, the third drug formulation may comprise at least one polymer, where at least one polymer in the third formulation may be the same and/as or different from at least one polymer in the first and second drug formulations. For example, the at least one polymer in the third formulation may provide a different release rate than provided by at least one polymer in the first and second drug formulations. In other examples, the at least one polymer in the third formulation provides substantially the same release rate as provided by at least one polymer in the first and second drug formulations.

In specific instances, the first, second, or optional third therapeutic compositions may comprise a plurality of drug different formulations for at least one drug. For example, a single drug type may be sequestered in formulations with polymers have different release rates and/or drug loadings, allowing further control of the drug release characteristics.

In yet another aspect, the present invention provides an implantable scaffold comprising a scaffold structure having a surface configured to be expanded in the patient's body. A first therapeutic composition comprising a first drug formulation including at least of a calcium chelating agent or EDTA complex, argatroban, at least one of apixaban and rivaroaxaban, and sirolimus is present in a polymer configured to rapidly release the first drug formulation into a vascular environment. A second therapeutic composition comprises a second drug formulation including at least argatroban, at least one of apixaban and rivaroaxaban, and sirolimus present in a polymer or other carrier configured for extended release of the second drug formulation into the vascular environment.

Carriers will typically comprise polymers, usually biodegradable polymers, more usually but not always being synthetic polymers synthesized from petroleum and other hydrocarbon feedstocks. Exemplary biodegradable, synthetic polymers are selected from the group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

An exemplary biodegradable polymer comprises poly(lactic-co-glycolic acid) (PLGA), where the PLGA is present at 5 μg to 15 μg per mm of scaffold structure length in the first therapeutic composition and from and from 5 μg to 20 μg per mm of scaffold structure length in the second therapeutic composition.

Alternatively, the polymer may comprises a non-degradable polymer, for example being selected from the group consisting of polyacrylates, polymethacrylates, poly(n-butyl methacrylate), poly(hydroxyethylmethacrylate), polyamides, nylons, nylon 12, dacron, polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers thereof.

In specific examples, the argatroban, at least one of apixaban and rivaroaxaban, and the sirolimus may be sequestered in a porous structure of the PLGA, and the release of the argatroban, the direct factor Xa inhibitor including at least one of apixaban and rivaroaxaban, and the sirolimus into the vascular environment occurs through a combination of diffusion and dissolution.

In a particular example, (1) a calcium chelating agent may be present in the first therapeutic composition at a concentration in a range from 2 μg to 15 μg per mm of scaffold structure length, the argatroban may be present in the first therapeutic composition at a concentration in a range from 0.5 μg to 3 μg per mm of scaffold structure length, the direct factor Xa inhibitor including at least one of apixaban and rivaroaxaban may be present at a concentration in a range from 0.5 μg to 3 μg per mm of scaffold structure length, and the sirolimus may be present at a concentration in a range from 0.5 μg to 3 μg per mm of scaffold structure length in the first therapeutic composition and (2) the argatroban may be present at a concentration in a range from 2 μg to 10 μg per mm of scaffold structure length, the direct factor Xa inhibitor including at least one of apixaban and rivaroxaban may be present at a concentration in a range from 2 μg to 10 μg per mm of scaffold structure length, and the sirolimus may be present at a concentration in a range from 2 μg to 10 μg per mm of scaffold structure length in the second therapeutic composition.

In a particular example, (1) the argatroban may be present in the first therapeutic composition at a concentration in a range from 0.5 μg to 3 μg per mm of scaffold structure length, the direct factor Xa inhibitor including at least one of apixaban and rivaroaxaban may be present at a concentration in a range from 0.5 μg to 3 μg per mm of scaffold structure length, and the sirolimus may be present at a concentration in a range from 0.5 μg to 3 μg per mm of scaffold structure length in the first therapeutic composition and (2) a calcium chelating agent may be present in the first therapeutic composition at a concentration in a range from 2 μg to 15 μg per mm of scaffold structure length, the argatroban may be present at a concentration in a range from 2 μg to 10 μg per mm of scaffold structure length, the direct factor Xa inhibitor including at least one of apixaban and rivaroxaban may be present at a concentration in a range from 2 μg to 10 μg per mm of scaffold structure length, and the sirolimus may be present at a concentration in a range from 2 μg to 10 μg per mm of scaffold structure length in the second therapeutic composition.

In other examples, the first therapeutic composition may be coated on one or more surfaces of the scaffold structure and the second therapeutic composition may be coated over at least a portion of the first therapeutic composition. For example, the first and second therapeutic compositions cover at least 75% of the area of inner and outer surfaces of the scaffold structure.

In a still further aspect, the present invention comprises a method for treating a vascular tissue injury in a patient. The method comprises expanding a scaffold structure at a target location in the patient's vasculature proximate a tissue injury. A first drug formulation including at least one of a drug selected from the group consisting of a calcium chelating agent, a direct factor IIa inhibitor and a direct factor Xa is released from a first therapeutic composition on the scaffold, and a second drug formulation including at least one drug selected from the group consisting of a calcium chelating agent, a direct factor IIa inhibitor and a direct factor Xa is released from a second therapeutic composition on the scaffold to the location of injury. The first therapeutic composition may be formulated to rapidly release the first drug formulation into a vascular environment, and the second therapeutic composition may be formulated to provide an extended release of the second drug formulation into the vascular environment.

In different instances of the methods of the present invention, the therapeutic compositions may be positioned on an external surface of the implantable scaffold, on an internal surface of the implantable scaffold, or on both external and internal surfaces of the implantable scaffold.

While the tissue injury will frequently be caused by expanding the scaffold at the location, in other cases the tissue injury may preexist, deploying the structure at the location.

In specific instances, at least one of the first drug formulation and the second drug formulation may comprise either or both or three of a calcium chelating agent, a direct factor IIa inhibitor and a direct factor Xa inhibitor.

In specific instances, the first (rapid release) drug formulation may release drug from the first therapeutic composition over a first time period is in a range from 3 hours to 28 days after implantation, usually from 3 hours to 7 days after implantation, preferably from 3 hours to 3 days after implantation, where the at least one drug of the first drug formulation is typically at a mean rate in the range from 1 μg/hour to 10 μg/hour, usually from 1 μg/hour to 5 μg/hour, preferably from 2 μg/hour to 4 μg/hour over a 24 hour period following exposure to the vascular environment, where the mean rate may be determined based on the amount (weight) of drug released over the total duration of the release.

In specific instances, the at least one drug of the second drug formulation is released from the second (sustained release) therapeutic composition over a second time period is in a range from 30 days to 12 months after implantation, usually from 30 days to 9 months after implantation, preferably from 30 days to 6 months after implantation, where the second therapeutic composition is typically configured to release the at least one drug of the second drug formulation for at a mean rate not exceeding 2 μg/hour, usually 1 μg/hour, preferably 0.5 μg/hour, and more preferably 0.1 μg/hour after the 24 hour period following exposure to the vascular environment, where the mean rate may be determined based on the amount (weight) of drug released over the total duration of the release.

In preferred instances, the therapeutic compositions are formulated to locally release the first and second drug formulation of a calcium chelating agent to the injury site at a rate or a concentration sufficient to begin to inhibit one or more of inflammation, cell proliferation, internal elastic lamina (IEL) injury, fibrin formation, and clot formation within about 1 hours to about 7 days after the structure is deployed.

In preferred instances, the therapeutic compositions are formulated to locally release the first and second drug formulation Xa to the injury site at a rate or a concentration sufficient to begin to inhibit one or more of inflammation, cell proliferation, internal elastic lamina (IEL) injury, fibrin formation, and clot formation within about 3 hours to about 7 days after the structure is deployed.

In other instances, the methods may further comprise releasing an antiplatelet drug from at least one of the first and second therapeutic compositions.

In another aspect, the present invention provides an implantable scaffold comprising a scaffold structure having a surface configured to be expanded in the patient's body. At least one therapeutic composition comprising a drug formulation including at least one drug selected from the group consisting of a calcium chelating agent, a direct factor IIa inhibitor and a direct factor Xa inhibitor s coated, layered, or otherwise bonded or affixed to the scaffold, wherein the therapeutic composition is formulated for an extended release of the drug formulation into a vascular environment.

Often, the drug formulation includes a calcium chelating agent, a direct factor IIa inhibitor and a direct factor Xa inhibitor, and in some instances, the drug formulation may include one or more additional drugs as described elsewhere herein.

Usually, the drug formulation comprises a polymer, and the drug(s) are incorporated into the polymer. The polymer is typically non-degradable in the vascular environment, where the drugs are loaded into a porous structure of the polymer and released by diffusion over an extended period. Alternatively, the polymer may be degradable in the vascular environment, and the drugs may be released by a combination of diffusion through and dissolution of the polymer.

Typically, the scaffold comprises a metal or a polymer which is non-degradable in the vascular environment, but in other instances the scaffold may be partly of wholly degradable, particularly when the polymer of the drug formulation is also degradable.

In specific examples, at least one drug of the drug formulation is released from the therapeutic composition over a time period of at least 28 days after implantation, usually at least 3 months after implantation, and preferably at least one year after implantation.

In other examples the therapeutic composition may be configured to release the at least one drug of the drug formulation at a mean rate not exceeding 2 μg/hour, usually 1 μg/hour, preferably 0.5 μg/hour, and more preferably 0.1 μg/hour following exposure to the vascular environment.

In many or all instances, the extended release of the drug formulation acts to accelerate dissolution of one or more of inflammation, cell proliferation, internal elastic lamina (IEL) injury, thrombin, fibrin formation, platelet aggregation, platelet activation, and clot or thrombus formation; and/or inhibit one or more of inflammation, cell proliferation, internal elastic lamina (IEL) injury, thrombin, fibrin formation, platelet aggregation, platelet activation, and clot or thrombus formation; and/or increase or prolong time before blood forms clot or thrombus.

In one aspect, a medical device may comprise a structure having at least one surface configured for internal use within a patient's body and a therapeutic composition comprising one or more active substances. These active substances include but not limited to a direct factor Xa inhibitor such as Apixaban, Betrixaban, Edoxaban. Otamixaban. Rivaroxaban, Razaxaban, (r)-n-(2-(4-(1-methylpiperidin-4-yl) piperazin-1-yl)-2-oxo-1-phenylethyl)-1h-indole-6-carboxamide (LY-517717), Daraxaban (YM-150)). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052) or others; and/or a direct IIa inhibitor such as Hirudin. Bivalirudin such as Angiomax. Desirudin. Lepirudin, atecegatran metoxil (AZD-0837). Argatroban. Dabigatran. Efegatran. Inogatran. Melagatran. Ximelagatran, or others: Vitamin K antagonist such as Acenocoumarol.

Coumatetralyl. Dicoumarol, Ethyl biscoumacetate. Phenprocoumon. Warfarin, Clorindione. Diphenadione. Phenindione. Tioclomarol, or others; and/or other anti-coagulant drug such as Antithrombin III. Defibrotide. Protein C (Drotrecogin alfa). Ramatroban. REGI, or others; and/or an antiplatelet drug such as Abciximab. Eptifibatide. Orbofiban. Roxifiban. Sibrafiban. Tirofiban. Clopidogrel. Prasugrel. Cangrelor. Elinogrel. Ticagrelor. Beraprost. Iloprost. Prostacyclin. Treprostinil. Acetylsalicylic acid/Aspirin. Aloxiprin. Carbasalate calcium. Indobufen. Triflusal. Dipyridamole/aspirin. Picotamide. Terbogrel. Terutroban. Cilostazol. Dipyridamole. Triflusal. Cloricromen. Ditazole. Vorapaxar. Ticlopidine, or others; and/or thrombolytic drugs/fibrinolytics drug such as Plasminogen activators r-tPA. Alteplase. Reteplase. Tenecteplase. Desmoteplase. Saruplase. Urokinase. Anistreplase. Monteplase. Streptokinase. Ancrod. Brinase. Fibrinolysin, or others; and/or Ethylenediaminetetraacetic acid (EDTA), calcium disodium edetate, magnesium dipotassium edetate, magnesium disodium edetate. Disodium edetate. Tetrasodium edetate. Trisodium edetate, monoammonium EDTA salt. Diammonium EDTA salt. Triammonium EDTA salt. Benzyldimethyltetradecylammonium EDTA salt. Tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA). 1,2-bis(2-aminophenoxy) ethane-N,N,N′,N′-tetraacetic acid (BAPTA), citrate, oxalate, the surfactant-EDTA complex, quaternary ammonium EDTA salt, benzalkonium EDTA salt. EDTA complex, salts, analogue, solvate, hydrate and derivative thereof; and/or an inhibitor for intrinsic pathway of coagulation and thrombosis such as a FX/FXIa inhibitor, protein Z-dependent protease inhibitor; and/or anti-proliferative drug such as Paclitaxel (Taxol), or salts, isomers, solvates, analogs, derivatives, metabolites, or prodrugs, and/or an m-TOR inhibitor such as sirolimus, biolimus, everolimus, myolimus, novolimus, ridaforolimus, temsirolimus, zotarolimus, or salts, isomers, solvates, analogs (including deuterated analogs), derivatives, metabolites, or prodrugs, and combinations thereof. In a preferred example, a medical device comprising a structure having at least one surface configured for internal use within a patient's body and a therapeutic composition comprising one or more active substances, wherein the one or more active substances comprises one of Apixaban. Rivaroxaban, or Argatroban. In a preferred example, a medical device comprising a structure having at least one surface configured for internal use within a patient's body and a therapeutic composition comprising one or more active substances, wherein the one or more active substances comprises Apixaban and Argatroban. Apixaban and an anti-platelet agent. Rivaroxaban and an anti-platelet agent, or Argatroban and an anti-platelet agent. In a preferred example, a medical device comprising a structure having at least one surface configured for internal use within a patient's body and a therapeutic composition comprising one or more active substances, wherein the one or more active substances comprises one of Apixaban or Rivaroxaban or an analogue thereof, and Argatroban or an analogue of it. In another preferred example, a medical device comprising a structure having at least one surface configured for internal use within a patient's body and a therapeutic composition comprising one or more active substances, wherein the one or more active substances comprises one of Apixaban or Rivaroxaban or an analogue thereof. Argatroban or its analogue, and one of Taxol or sirolimus or an analogue thereof analogues.

In one aspect, a medical device may comprise a structure having an external surface configured for internal use within a patient's body and a therapeutic composition comprising one or more active substances including a calcium chelating agent disposed on at least one surface, preferably disposed on the entire external surface of the structure. In some examples, the external surface of the structure is configured to be positioned adjacent to an injury site in the patient's body, preferably expanding such site to a larger configuration. In some examples, the therapeutic composition is formulated to locally release the one or more active substances to the injury site at a rate sufficient to generate a tissue concentration of about 2 ng/mg tissue to about 50 ng/mg tissue of the one or more active substances at the injury site within about 3 hours after the external surface of the structure is positioned adjacent the injury site.

In one aspect, a medical device may comprise a structure having an external surface configured for internal use within a patient's body and a therapeutic composition comprising one or more active substances including a direct factor IIa inhibitor disposed on at least one surface, preferably disposed on the entire external surface of the structure. In some examples, the external surface of the structure is configured to be positioned adjacent to an injury site in the patient's body, preferably expanding such site to a larger configuration. In some examples, the therapeutic composition is formulated to locally release the one or more active substances to the injury site at a rate sufficient to generate a tissue concentration of about 2 ng/mg tissue to about 200 ng/mg tissue of the one or more active substances at the injury site within about 3 hours after the external surface of the structure is positioned adjacent the injury site.

In some examples, the therapeutic composition is formulated to reduce, inhibit, and/or maintain reduced cell inflammatory at implantation injury site for an extended period, usually at least about 28 days after the external surface of the structure is positioned adjacent the injury site, typically from 1 month to about 12 months.

While the drug carriers of the present invention will often be synthetic, biodegradable polymers, as described above, in other examples, the drug carrier may be formulated with other biocompatible, biodegradable or non-biodegradable materials, including non-synthetic polymers, such as biological polymers, such as proteins, polypeptides, nucleic acids, carbohydrates, and the like, formulated in coating or other sequestration materials.

Such biological and other biocompatible coatings can reduce the foreign body inflammatory response induced by the intraluminal device. In some examples, such biocompatible drug carriers can deliver effective drug concentration within the vessel walls, provide a reservoir for abluminal drug elution, direct a drug toward a vessel wall, enable enhanced drug-tissue permeation to achieve enhanced drug bioavailability, improve homogeneous drug distribution, and/or improve drug stability.

In some examples, the therapeutic composition is formulated to improve drug delivery to target cells, such as diabetic cells.

In some examples, the therapeutic composition is formulated to improve drug lipophilicity and carrier hydrophobicity to facility drug effective delivery to vessel wall and extend drug release rate.

In some examples, instead of being a component of the coating, the therapeutic agent may also be chemically combined with the coating, carrier, or matrix by any chemical combination technique.

In some examples, the therapeutic composition is formulated with a biocompatible carrier having a surface-binding cell adhesion polypeptide deposited on the stent surface forming an amino-containing hydrophobic bond by binding moiety included but not limit to 3,4-dihydroxyphenylalanine (DOPA) or having adhesives peptide or polypeptide such as in the form of L-DOPA-containing proteins. These positive charged amino-terminal region polypeptides inhibit platelet activation and degranulation and limit platelet adhesion in the stent surface.

In some examples, the therapeutic composition is formulated with a biocompatible carrier deposited on the surface, forming a hydrophobic coating to enhance the corrosion resistance, avoiding the aggregation of platelets in the blood vessels and appropriate proliferation of endothelial cells and controlled proliferation of smooth muscle cells, which reduces the development of pathology, such as neointimal hyperplasia, thrombosis, and restenosis.

In some examples, the therapeutic composition is formulated with a biocompatible carrier with “mussel-inspired.” catechol-functionalized hydrogels. In some examples, the therapeutic composition is formulated with a biocompatible hydrogel or soft gel carrier when it contacted with body fluid, the coating will reduce, inhibit, and/or maintain reduced cell inflammatory at the injury site and extend drug release rate. In some examples, the therapeutic composition is formulated with polyunsaturated fatty acids (PUFAs) such as Omega-3 or Omega-6 to modify platelet responsiveness with anticoagulants.

The biocompatible drug carriers include but not limit to low water solubility amino acid, peptide, polypeptide, modified peptide conjugated with a linker or a spacer, modified polypeptide conjugated with a linker or a spacer, fatty acid including Omega-3 or Omega-6 polyunsaturated fatty acids, crosslinked fatty acid, crosslinked oil, including fish oil. Vitamin E, hazelnut oil, avocado oil, macadamia nut oil, grapeseed oil, groundnut oil (peanut oil), sesame oil, corn oil, almond oil, sunflower oil, hemp oil, tea-oil camellia, pectin and gelatin. Exemplary amino acids are selected from the group consisting of phenylalanine, valine, threonine, tryptophan, methionine, leucine, isoleucine, lysine, histidine, arginine, cysteine, glycine, glutamine, proline, tyrosine, alanine, aspartic acid, asparagine, glutamic acid, serine, and selenocysteine, and derivatives and combinations thereof. Exemplary low-solubility amino acid having a solubility in unbuffered water of less than 40 mg/mL are selected from the group consisting of asparagine, aspartic acid, cystine, eptifibatide, isoleucine, leucine, methionine, phenylalanine, tryptophan, tyrosine, and combinations thereof. Exemplary of peptides include but not limit to lysine, ornithine, arginine, histidine, glutamic acid, aspartic acid, histidine, polyornithine, serine, threonine, tyrosine, leucine, analogues including D and L isomers, oligomers, copolymers, block polymers, derivatives, and any peptide with different amino acid sequence. Exemplary of peptides include but not limit to signaling peptides, carrier peptides, enzyme-inhibiting peptides, neurotransmitter-inhibiting peptides, antimicrobial peptides, analogs, derivatives, and combinations thereof, Exemplary of polypeptides include but not limit to poly(lysine), poly(ornithine), poly(arginine), poly(histidine), poly(glutamic acid), poly(aspartic acid), poly(histidine), poly(ornithine), poly(serine), poly(threonine), poly(tyrosine), poly(leucine), analogues including D and L isomers, copolymers, block polymers, derivatives, and combinations thereof. Exemplary of cell adhesion polypeptides include but not limit to fibronectin, vitronectin, laminin, elastin, fibrinogen, and collagens, such as types I, II, and V, and any peptide derived from any of the peptides, including a cell adhesive peptide fragment having the amino acid sequence, and any peptide with different amino acid sequence. Exemplary of drug carries are containing at least one multiple bonds, i.e, preferably one unsaturated fatty acid moiety, fatty acids, cross-linked fatty acid, fatty acid esters, fatty acid derivatives, ethers, diethers, tetraethers, lipids, oils, fats, glycerides, tri-glycerides, glycol esters, glycerin esters, fish oil or derivatives thereof, vitamin E or derivatives thereof, peanut oil, cotton-seed oil, oleic acid or combinations thereof as well as mixtures of the aforementioned substances. Suitable saturated fatty acids include but not limited to, those selected from the group consisting of butyric acid, valeric acid caproic acid, enanthic acid, caprylic acid, pelargonic acid, capric acid, lauric acid, myristic acid, pentadecanoic acid, palmitic acid, margaric acid, stearic acid, arachidic acid, behenic acid, lignoceric acid, cerotic acid, linoleic acid, oleic acid and stearic acid. Suitable unsaturated fatty acids include but not limited to those selected from the group consisting of arachidonic acid, oleic acid, erucic acid, nervonic acid, linolenic acid, arachidonic acid, eicosapentanoic acid (EPA), docosahexanoic acid (DHA), palmitoleic acid and myristoleic acid.

In some examples, the biocompatible carrier comprises polypeptides conjugated with an adhesive moiety and/or a linker to enhance hydrophobicity and ease drug delivery. Adhesive moieties, including but not limited to catechol moieties and L-DOPA-containing proteins, may be joined to each other and to cell adhesive polypeptides by linkers including but not limit to hyaluronic acid, polyethylene glycol/poly lysine, dopamine. 1,6-diaminohexane. 1,5-diaminopentane. 1,4-diaminobutane, or 1,3-diaminopropane or any compound having at least two hydroxyl group or two amine group that could reacted with polypeptide amnio acid group. In some examples, the biocompatible carrier is peptides and conjugated with a spacer and/or a linker to make peptide more hydrophobic and having controlled delivery of therapeutic compounds or an extended-release rate of therapeutic compounds. In some examples, the biocompatible carrier is a controlled release layer contains one or more matrix forming gelling agents selected from group consisting of hydroxypropyl methylcellulose, methylcellulose, hydroxypropyl cellulose, carbomer, carboxy methylcellulose, gum tragacanth, gum acacia, guar gum, pectin, modified starch derivatives, xanthan gum, locusta bean gum, sodium alginate, which on contact with gastric fluid swells and gels, forming matrix structure that entraps the gas released and also releases the active agent in a controlled manner. In some examples, the biocompatible carrier is an absorption promoter selected from the group consisting of propylene glycol, propylene glycol monolaurate, isopropyl palmitate, 1,2,6-hexanetriol, polyethylene glycol, diisopropyl adipate, polyethylene glycol 400 acetate, and ethylene glycol monoether. In some examples, the therapeutic composition comprises a coating disposed on the external surface of the structure, and the coating comprises plasmid DNA loaded biodegradable polymer such as Polylactic-polyglycolic acid (PLGA) as stent coating. This gene therapy on the vessel wall by effective transfection of neointimal cells by local delivery of DNA. The carriers include but not limit to DNA fragments, nucleic acids, genetic material, oligonucleotides, radioisotopes, or combinations of these classes of compounds.

For example, the therapeutic composition is preferably formulated to locally release the one or more active substances to the injury site at a rate sufficient to generate a tissue concentration of about 20 ng/mg tissue to about 200 ng/mg tissue, or more preferably about 40) ng/mg tissue to about 200 ng/mg tissue, of the one or more active substances at the injury site within about 3 hours after the external surface of the structure is positioned adjacent the injury site. In another example of this aspect, the medical device structure has an internal (inner) surface, wherein one or more agents are coated on at least one region of the inner structure surface, preferably coated on the entire inner structure surfaces. In yet another example of this aspect, the medical device structure has more than two surfaces, and wherein the one or more agents are coated on all or some of these surfaces. In yet another example of this aspect, the coating thickness may be uniform between surfaces or vary between surfaces of the structure. In yet another example of this aspect, the device may have a partial or full covering or a sleeve on one or more surfaces of the device (such as PTFE. Dacron, or other type material) wherein said material comprises the one or more agents. In some examples, the therapeutic composition is formulated to release substantially all of the one or more active substances within about 1 to about 90 days. In some examples, the therapeutic composition is formulated to release substantially all of the one or more active substances about 90 to about 180 days or more. In some examples, the therapeutic composition is formulated to locally release the one or more active substances to the injury site at a rate sufficient to generate a tissue concentration of 0.1 ng/mg or more for a period ranging from 3 hours to 90 days, 3 hours to 180 days, or 3 hours to 270) days or more.

In some examples, the therapeutic composition is formulated to release the one or more agents, configured to release the two or more agents, or is configured to release the three or more agents, in one or more of the following: a burst release phase and an extended release phase, wherein the release of a first phase comprises a faster release rate than a second release phase, or other.

In some examples, the therapeutic composition is formulated to release the one or more agents, wherein the therapeutic composition comprises a first therapeutic composition formulated to release said agents at a faster rate, and a second therapeutic composition formulated to release said agents at a slower release rate.

In some examples, the device comprises one therapeutic composition formulated to release one or more of calcium chelating agent such as of a calcium chelating agent, direct factor Xa inhibitor, direct factor IIa inhibitor, and/or an anti-proliferative, wherein the formulation formulated to release the drugs over an extended period ranging from 7 days to 6 months, preferably ranging from 14 days to 6 months, more preferably ranging from 21 days to 6 months, and most preferably ranging from 30 days to 1 year from exposure to vascular environment. Optionally, the formulation is configured to have a bolus drug release rate within the first 1 hour, 3 hours, or first 24 hours, from exposure to vascular environment.

In some examples, the therapeutic composition is formulated to release the one or more active substances at a rate sufficient to generate a tissue concentration at the injury site within a range of about 2 ng/mg to about 800 ng/mg within about 3 hours. In some examples, the therapeutic composition is formulated to release the one or more active substances at a rate sufficient to generate a tissue concentration at the injury site within a range of about 10 ng/mg to about 100 ng/mg within about 3 hours.

In some examples, the therapeutic composition is formulated to release the one or more active substances at a rate sufficient to generate a tissue concentration at the injury site within a range of about 2 ng/mg to about 100 ng/mg within about 24 hours. In some examples, the therapeutic composition is formulated to release the one or more active substances at a rate sufficient to generate a tissue concentration at the injury site within a range of about 3 ng/mg to about 50) ng/mg within about 24 hours. In some examples, the therapeutic composition is formulated to release the one or more active substances at a rate sufficient to generate a tissue concentration at the injury within a range of about 4 ng/mg to about 25 ng/mg within about 24 hours.

In some examples, the therapeutic composition is formulated to release the one or more active substances at a rate sufficient to generate a tissue concentration at the injury site within a range of about 1 ng/mg to about 30 ng/mg within about 7 days. In some examples, the therapeutic composition is formulated to release the one or more active substances at a rate sufficient to generate a tissue concentration at the injury site within a range of about 1.5 ng/mg to about 20 ng/mg within about 7 days. In some examples, the therapeutic composition is formulated to release the one or more active substances at a rate sufficient to generate a tissue concentration at the injury site within a range of about 2 ng/mg to about 25 ng/mg within about 7 days.

In some examples, the therapeutic composition is formulated to release the one or more active substances at a rate sufficient to generate a tissue concentration at the injury site within a range of about 0.5 ng/mg to about 30 ng/mg within about 28 days. In some examples, the therapeutic composition is formulated to release the one or more active substances at a rate sufficient to generate a tissue concentration at the injury site within a range of about 1 ng/mg to about 20 ng/mg within about 28 days. In some examples, the therapeutic composition is formulated to release the one or more active substances at a rate sufficient to generate a tissue concentration at the injury site within a range of about 1.5 ng/mg to about 25 ng/mg within about 28 days.

In some examples, the therapeutic composition is formulated to release the one or more active substances at a rate sufficient to generate a tissue concentration at the injury site within a range of about 0.1 ng/mg to about 10 ng/mg within about 90 days or about 180 days.

In some examples, the therapeutic composition is formulated to release the one or more active substances at a rate sufficient to generate a tissue concentration at a location proximal or distal a proximal end of the structure or a distal end of the structure, respectively, within a range of about 0.5 ng/mg to about 500 ng/mg within about 3 hours. In some examples, the therapeutic composition is formulated to release the one or more active substances at a rate sufficient to generate a tissue concentration at the location proximal or distal the proximal end of the structure or the distal end of the structure, respectively, within a range of about 1 ng/mg to about 35 ng/mg within about 3 hours. In some examples, the therapeutic composition is formulated to release the one or more active substances at a rate sufficient to generate a tissue concentration at the location proximal or distal the proximal end of the structure or the distal end of the structure, respectively, within a range of about a range of about 1.5 ng/mg to about 30 ng/mg within about 3 hours. In some examples, the therapeutically effective dose is sufficient to generate a tissue concentration of the direct factor IIa inhibitor of about 0.001 ng/g tissue to about 100 mg/g tissue, preferably, the therapeutically effective dose is sufficient to generate a tissue concentration of the direct factor IIa inhibitor of about 0.01 ng/g tissue to about 100 mg/g tissue, more preferably, the therapeutically effective dose is sufficient to generate a tissue concentration of the direct factor IIa inhibitor of about 0.1 ng/g tissue to about 100 mg/g tissue. For example, the therapeutically effective dose may be sufficient to generate a tissue concentration of the direct factor IIa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue: in about 1 day. 30 days. 60 days, 90 days, or 120 days after introducing the therapeutically effective dose. In some examples, the therapeutically effective dose is sufficient to generate a blood concentration of the direct factor IIa inhibitor which is less than about 200 ng/ml. 100 ng/ml. 50 ng/ml 25 ng/ml, or 10 ng/ml. In some examples, the therapeutically effective dose is sufficient to generate a blood concentration of the direct factor IIa inhibitor which is less than a systemic therapeutic concentration of the direct factor IIa inhibitor for any systemic indication. In some examples, the therapeutically effective dose is sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory ophthalmic condition or disease for more than about 6 hours to about 3 days.

In some examples, the therapeutically effective dose is sufficient to maintain a tissue concentration of the direct factor IIa inhibitor of about 0.1 ng/g tissue to about 100 mg/g tissue for about 1 day to about 120 days, for about 1 day to about 1 year. 30 days to about 1 year. 3 months to about 1 year, or 6 months to about 1 year.

In some examples, the therapeutic composition comprises at least three therapeutic active substances comprising a calcium chelating agent, a direct factor Xa inhibitor and a direct factor IIa inhibitor.

In some examples, the therapeutic composition comprising a factor Xa inhibitor further comprises at least one additional therapeutically active substance. In some examples, the at least one additional therapeutically active substance comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban, or a salt, isomer, solvate, analog, derivative, metabolite, or prodrugs thereof. In some examples, the direct factor Xa inhibitor comprises apixaban and the direct factor IIa inhibitor comprises argatroban. In some examples, the therapeutically effective dose of the direct factor IIa inhibitor is within a range of about 50 micrograms to about 10 mg. In some examples, the therapeutically effective dose is sufficient to generate a tissue concentration of the direct factor IIa inhibitor of about 0.1 ng/g tissue to about 100 mg/g tissue. For example, the therapeutically effective dose may be sufficient to generate a tissue concentration of the direct factor IIa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue. In some examples, the therapeutically effective dose is sufficient to generate a blood concentration of the direct factor IIa inhibitor which is less than about 200 ng/ml. 100 ng/ml. 50 ng/ml 25 ng/ml, or 10 ng/ml. In some examples, the therapeutically effective dose is sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory ophthalmic condition or disease. In some examples, the therapeutically effective dose is sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory ophthalmic condition or disease for more than about 6 hours to about 3 days. In some examples, the therapeutically effective dose is sufficient to maintain a tissue concentration of the direct factor IIa inhibitor of about 0.1 ng/g tissue to about 100 mg/g tissue for about 1 day to about 1 year. 30 days to about 1 year. 3 months to about 1 year, or 6 months to about 1 year. In some examples, the weight compositional ratio of the direct factor Xa inhibitor to the direct factor IIa inhibitor in the therapeutic composition is within a range of about 3:1 to about 1:3. For example, the weight compositional ratio of the direct factor Xa inhibitor to the direct factor IIa inhibitor in the therapeutic composition may be about 1:1.

In some examples, the therapeutic composition comprises one or more anticoagulant agents that has an IC50 to inhibit factor Xa and factor II at a dose ranging from 0.0001 nM to 1000 nM, preferably at a dose ranging from 0.0001 nM to 100 nM, more preferably at a dose ranging from 0.0001 nM to 10 nM, and most preferably at a dose ranging from 0.0001 nM to 1 nM.

In some examples, the therapeutic composition is formulated to release the one or more active substances at a rate sufficient to generate a tissue concentration of about 0.5 ng/mg to about 10 ng/mg of tissue adjacent to the device structure within about 28 days or about 90 days or about 180 days.

In some examples, the therapeutic composition is formulated to release the direct factor Xa inhibitor and the anti-proliferative agent at the same rate. In some examples, the therapeutic composition is formulated to release the direct factor Xa inhibitor, and the anti-proliferative agent at different rates. In other examples, the therapeutic composition is formulated to release the direct factor Xa inhibitor at a faster rate than the anti-proliferative agent within the first 3 hours, 1 day, or 72 hour. In yet another example, the therapeutic composition is formulated to release the direct factor Xa inhibitor at a slower rate than the anti-proliferative agent within the first 3 hours, 1 day, or 72 hour.

In some examples, the release rate ratio of the direct factor Xa inhibitor to the anti-proliferative agent is within a range of about 3:2 to about 6:1, or about 2.2:2 to about 6:1, or about 2.5:2 to about 6:1. In some examples, the release rate ratio of the direct factor Xa inhibitor to the anti-proliferative agent is within a range of about 3:2 to about 6:1, about 2.2:2 to about 6:1, or about 2.5:2 to about 6:1 within about 3 hours, about 24 hours, about 7 days, or about 28 days. In some other examples, the release rate ratio of the direct factor Xa inhibitor to the anti-proliferative agent is within a range of about 1:1 to about 2:1 within about 3 hours, 1 day, about 3 days, about 7 days, or about 28 days.

In some examples, the therapeutic composition is formulated to release the anti-proliferative agent at a rate of about 1 μg/second/mm device to about 50 μg/day/mm device, of about 1 μg/min/mm device to about 10 μg/day/mm device, or of about 1 μg/hour/mm device to about 7 μg/day/mm device within about 3 hours, about 1 day, or about 3 days.

In some examples, the therapeutic composition is formulated to release the anti-proliferative agent at a rate of about 1 μg/hour/mm device to about 4 μg/day/mm device.

In some examples, the weight compositional ratio of the direct factor Xa inhibitor to the anti-proliferative agent in the therapeutic composition is about 5:2, about 2:1, about 1.25:1, or about 1:1. In some examples, the weight compositional ratio of the direct factor Xa inhibitor to the anti-proliferative agent in the therapeutic composition is within a range of about 5:1 to about 3:1 or about 5:1 to about 1:1.

In some examples, the therapeutic composition comprises a coating disposed on one or more surfaces of the device structure, and the coating comprises a first layer and a second layer. In some examples, the first layer comprises the direct factor Xa inhibitor. In some examples, the first layer comprises the anti-proliferative agent and the second layer comprises the direct factor Xa inhibitor. In some examples, the therapeutic composition further comprises a top layer or coat of the same or different material as the first layer or the second layer. In some examples, the first layer comprises the direct factor Xa inhibitor and the anti-proliferative agent. In some examples, the second layer comprises a top layer or coat of the same or different material as the first layer. In some examples, the therapeutic composition comprises a coating disposed on one or more surfaces the device structure, and the coating further comprises a biodegradable polymer carrier. In some examples, the first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers maybe the same or different.

In some examples, the weight compositional ratio of the biodegradable polymer carrier to the one or more active substances is about 1:5 to about 3:2, about 0.5:1 to about 1:1, or about 1:5 to about 1.25:1. In a preferred example, the polymer is biodegradable.

In some other examples, the weight compositional ratio of the carrier to the one or more active substances is about 1:5 to about 3:2, about 0.5:1 to about 1:1, or about 1:5 to about 1.25:1. In one example the carrier is one or more excipients.

In some examples, the therapeutic composition is disposed on at least one surface of the device, preferably on at least the external and/or the inner surfaces of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the therapeutic composition comprises a coating disposed on the external surface of the structure, and the coating further comprises a non-degradable polymer carrier. In some examples, the therapeutic composition comprises a coating disposed on the external surface of the structure, and the coating comprises at least one layer of a polymeric material containing the direct factor Xa inhibitor. In some examples, the therapeutic composition comprises a coating disposed on the external surface of the structure, and the coating consists of a single layer of a polymeric material which releasably contains the direct factor Xa inhibitor. In some examples, the therapeutic composition further comprises a top layer or coat comprising the same or different polymeric material. In some examples, the direct factor Xa inhibitor is uniformly distributed in the polymeric material. In some examples, the direct factor Xa inhibitor is non-uniformly distributed in the polymeric material.

In some examples, the therapeutic composition comprises a coating disposed on at least on surface of the structure, and the coating comprises at least one layer of a polymeric material holding one or more of the direct factor Xa inhibitor and the anti-proliferative agent. In some examples, the therapeutic composition comprises a coating disposed on the external surface of the structure, and the coating consists of a single layer of a polymeric material which releasably contains the direct factor Xa inhibitor and the anti-proliferative agent. In some examples, the therapeutic composition further comprises a top layer or coat comprising the same or different polymeric material. In some examples, the direct factor Xa inhibitor, and the anti-proliferative agent are uniformly distributed in the polymeric material. In some examples, the direct factor Xa inhibitor, and the anti-proliferative agent are non-uniformly distributed in the polymeric material. In some examples, the one or more active substances is present in the polymeric material at weight ratios within a range of about 1:1 to about 6:1 of direct factor Xa inhibitor to anti-proliferative agent.

In some examples, the polymeric material is porous. In some examples, the polymeric material has a porosity within a range of about 10 nm to about 10 μm. In some examples, the polymeric material is non-degradable. In some examples, the polymeric material is biodegradable. In some examples, the polymeric material has a degradation rate within a range of about 1 month to about 36 months. In some examples, the polymeric material comprises a material selected from a group consisting of polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-&-caprolactone) (including 70:30 to 99:1 PLA-co-PCL, such as 90:10 PLA-co-PCL), poly(glycolide-co-8-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide. D-lactide and D,L-lactide. In some examples, the polymeric material comprises a material selected from a group of non-degradable polymeric materials consisting of polyacrylates, polymethacrylates, poly(n-butyl methacrylate), poly(hydroxyethylmethacrylate), polyamides, nylons, nylon 12. Dacron. Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

In some examples, the therapeutic composition is disposed within a drug reservoir fluidly coupled to the external surface of the structure.

In another aspect, a medical device may comprise a structure having at least one surface configured for internal use within a patient's body and a therapeutic composition comprising two or more active substances including a calcium chelating agent, a direct factor Xa inhibitor and a direct factor IIa inhibitor. In some examples, the at least one surface of the structure is configured to be positioned adjacent an injury site in the patient's body. In some examples, the therapeutic composition is formulated to locally release the two or more active substances to the injury site at a rate or a concentration sufficient to reduce cell proliferation at the injury site within about 3 hours to about 7 days, or within about 28 days to about 12 months, after the external surface of the structure is positioned adjacent the injury site. For example, the therapeutic composition is preferably formulated to locally release the two or more active substances to the injury site at a rate sufficient to generate a tissue concentration of about 20 ng/mg tissue to about 200 ng/mg tissue, or more preferably about 40 ng/mg tissue to about 200 ng/mg tissue, of the one or more active substances at the injury site within about 3 hours after the external surface of the structure is positioned adjacent the injury site. In some examples, the therapeutic composition is formulated to locally release the two or more active substances to the injury site at a rate sufficient to generate a tissue concentration of about 2 ng/mg tissue to about 200 ng/mg tissue of the one or more active substances at the injury site within about 3 hours after the external surface of the structure is positioned adjacent the injury site. For example, the therapeutic composition is preferably formulated to locally release the one or more active substances to the injury site at a rate sufficient to generate a tissue concentration of about 20 ng/mg tissue to about 200 ng/mg tissue, or more preferably about 40 ng/mg tissue to about 200 ng/mg tissue, of the one or more active substances at the injury site within about 3 hours after the external surface of the structure is positioned adjacent the injury site.

In some examples, the therapeutic composition further comprises an anti-proliferative agent. In some examples, the direct factor IIa inhibitor comprises argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises argatroban, or a salt, isomer, solvate, analog (including deuterated analog), derivative, metabolite, or prodrugs thereof. In some examples, the direct factor IIa inhibitor comprises dabigatran, or a salt, isomer, solvate, analog (including deuterated analog), derivative, metabolite, or prodrugs thereof.

In some examples, the direct factor Xa inhibitor comprises apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In some examples, the direct factor Xa inhibitor comprises rivaroxaban, or a salt, isomer, solvate, analog (including deuterated analog), derivative, metabolite, or prodrugs thereof. In some examples, the direct factor Xa inhibitor comprises apixaban, or a salt, isomer, solvate, analog (including deuterated analog), derivative, metabolite, or prodrugs thereof. In some examples, the anti-proliferative agent comprises Paclitaxel (Taxol), or salts, isomers, solvates, analogs, derivatives, metabolites, or prodrugs thereof. In some examples, the anti-proliferative agent comprises an m-TOR inhibitor. In some examples, the anti-proliferative agent comprises sirolimus, biolimus, everolimus, myolimus, novolimus, ridaforolimus, temsirolimus, zotarolimus, or salts, isomers, solvates, analogs (including deuterated analogs), derivatives, metabolites, or prodrugs thereof. In some examples, the anti-proliferative agent comprises sirolimus, or a salt, isomer, solvate, analog (including deuterated analog), derivative, metabolite, or prodrugs thereof.

In some examples, the direct factor IIa inhibitor comprises Argatroban and the direct factor Xa inhibitor comprises apixaban. In some examples, the direct factor IIa inhibitor comprises Argatroban, the direct factor Xa inhibitor comprises apixaban, and the anti-proliferative agent comprises sirolimus. In yet another examples the therapeutic composition comprises one of Apixaban. Rivaroxaban, or an analogue thereof, and one of Sirolimus or an analogue of Sirolimus.

In some examples, the therapeutic composition of a calcium chelating agent, a direct factor IIa inhibitor and a direct factor Xa inhibitor is formulated to reduce cell proliferation compared to either the direct factor IIa inhibitor or the direct factor Xa inhibitor alone. In some examples, the therapeutic composition is formulated to reduce, inhibit, and/or maintain reduced cell proliferation at the injury site at about 28 days after the external surface of the structure is positioned adjacent the injury site to about 12 months. In some examples, the therapeutic composition is formulated to reduce smooth muscle cell proliferation at the injury site. In some examples, the therapeutic composition of a calcium chelating agent, a direct factor IIa inhibitor and a direct factor Xa inhibitor and an antiproliferative is formulated to reduce cell proliferation compared to an antiproliferative alone. In some examples, the therapeutic composition is formulated to reduce, inhibit, and/or maintain reduced cell proliferation at the injury site at about 28 days after the external surface of the structure is positioned adjacent the injury site to about 12 months

In other examples, the therapeutic composition comprising a calcium chelating agent, a direct factor IIa inhibitor and a direct factor Xa inhibitor is formulated to release said agents at a rate and/or concentration sufficient to accelerate dissolution or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a calcium chelating agent, a direct factor IIa inhibitor and a direct factor Xa inhibitor is formulated to release said agents to accelerate dissolution of or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a calcium chelating agent, a direct factor IIa inhibitor and a direct factor Xa inhibitor formulated to have a weight composition ratio of factor Xa inhibitor to factor IIa inhibitor in the ratio ranging from about 1:1:1 to about 10:1:1 In other examples, the therapeutic composition comprising a calcium chelating agent, a direct factor IIa inhibitor and a direct factor Xa inhibitor formulated to have a weight composition ratio of factor Xa inhibitor to factor IIa inhibitor in the ratio ranging from about 0.5:1 to about 5:1.

In some examples, the therapeutic composition is formulated to reduce one or more of cell proliferation or fibrin formation within 7 days or longer.

In some examples, the therapeutic composition is formulated to release the two or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device. In some examples, the therapeutic composition is formulated to begin releasing the two or more active substances prior to positioning of the device adjacent to the injury site, or immediately after, or within about 5, about 15, or about 30 minutes after the at least one surface of the structure is positioned adjacent the injury site. In some examples, the therapeutic composition is formulated to begin releasing the two or more active substances before the external surface of the structure is positioned adjacent the injury site. In some examples, the therapeutic composition is formulated to release substantially all of the two or more active substances within about 1 to about 90 days or more. In some examples, the therapeutic composition is formulated to release substantially all of the two or more active substances within about 90 to about 180 days or more. In some examples, the therapeutic composition is formulated to release substantially all of the two or more active substances within about 7 days or about 28 days. In some examples, the therapeutic composition is formulated to release substantially all of the two or more active substances within about 3 hours or about 6 hours or about 12 hours or about 1 day or about 3 days. In some examples, the therapeutic composition is formulated to release at least 50% or at least 60% or at least 70% of the two or more active substances within about 3 hours or about 6 hours or about 12 hours or about 1 day or about 3 days or about 7 days or about 28 days.

In some examples, the therapeutic composition is formulated to release the two or more active substances at a rate sufficient to generate a tissue concentration at the injury site within a range of about 2 ng/mg to about 100 ng/mg within about 24 hours. In some examples, the therapeutic composition is formulated to release the two or more active substances at a rate sufficient to generate a tissue concentration at the injury site within a range of about 3 ng/mg to about 50 ng/mg within about 24 hours. In some examples, the therapeutic composition is formulated to release the two or more active substances at a rate sufficient to generate a tissue concentration at the injury within a range of about 4 ng/mg to about 25 ng/mg within about 24 hours.

In some examples, the therapeutic composition is formulated to release the two or more active substances at a rate sufficient to generate a tissue concentration at the injury site within a range of about 1 ng/mg to about 30 ng/mg within about 7 days. In some examples, the therapeutic composition is formulated to release the two or more active substances at a rate sufficient to generate a tissue concentration at the injury site within a range of about 1.5 ng/mg to about 20 ng/mg within about 7 days. In some examples, the therapeutic composition is formulated to release the two or more active substances at a rate sufficient to generate a tissue concentration at the injury site within a range of about 2 ng/mg to about 25 ng/mg within about 7 days.

In some examples, the therapeutic composition is formulated to release the two or more active substances at a rate sufficient to generate a tissue concentration at the injury site within a range of about 0.5 ng/mg to about 30 ng/mg within about 28 days. In some examples, the therapeutic composition is formulated to release the two or more active substances at a rate sufficient to generate a tissue concentration at the injury site within a range of about 1 ng/mg to about 20 ng/mg within about 28 days. In some examples, the therapeutic composition is formulated to release the two or more active substances at a rate sufficient to generate a tissue concentration at the injury site within a range of about 1.5 ng/mg to about 25 ng/mg within about 28 days.

In some examples, the therapeutic composition is formulated to release the two or more active substances at a rate sufficient to generate a tissue concentration at the injury site within a range of about 0.1 ng/mg to about 10 ng/mg within about 90 days or about 180 days.

In some examples, the release rate ratio of the direct factor IIa inhibitor to the direct factor Xa inhibitor to the anti-proliferative agent is within a range of about 1:1:1 to about 4:4:1. In some examples, the therapeutic composition is formulated to release the direct factor IIa inhibitor at a rate of about 4 μg/hour/mm device to about 14 μg/day/mm device. In some examples, the therapeutic composition is formulated to release the direct factor Xa inhibitor at a rate of about 4 μg/hour/mm device to about 14 μg/day/mm device. In some examples, the therapeutic composition is formulated to release the anti-proliferative agent at a rate of about 1 μg/hour/mm device to about 4 μg/day/mm device.

In some examples, the weight compositional ratio of the direct factor IIa inhibitor to the direct factor Xa inhibitor in the therapeutic composition is about 1:1. In some examples, the weight compositional ratio of the direct factor IIa inhibitor to the direct factor Xa inhibitor in the therapeutic composition is within a range of about 3:1 to about 1:3, for example about 1:1. In some examples, the weight compositional ratio of the direct factor IIa inhibitor to the direct factor Xa inhibitor to the anti-proliferative agent in the therapeutic composition is about 5:5:2. In some examples, the weight compositional ratio of the direct factor IIa inhibitor to the direct factor Xa inhibitor to the anti-proliferative agent in the therapeutic composition is within a range of about 6:6:1 to about 1:3:1.

In some other examples, the weight compositional ratio of the carrier to the two or more active substances is about 1:5 to about 3:1, about 0.5:1 to about 1:1, or about 1:5 to about 1.25:1. In one example the carrier is one or more excipients.

In some examples, the therapeutic composition is disposed on the external surface of the structure and on the internal (inner) surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the therapeutic composition comprises a coating disposed on at least one surface of the structure, and the coating comprises a first layer and a second layer. In some examples, the first layer comprises the direct factor IIa inhibitor and the direct factor Xa inhibitor. In some examples, the first layer comprises the direct factor IIa inhibitor and the second layer comprises the direct factor Xa inhibitor. In some examples, the therapeutic composition further comprises a top layer or coat of the same or different material as the first layer or the second layer.

In some examples, the therapeutic composition comprises a coating disposed on at least one surface of the structure, and the coating comprises a first layer and a second layer. In some examples, the first layer comprises the anti-proliferative agent, the direct factor IIa inhibitor, and the direct factor Xa inhibitor. In some examples, the second layer comprises a top layer or coat of the same or different material as the first layer. In some examples, the first layer comprises the anti-proliferative agent and the second layer comprises the direct factor IIa inhibitor and the direct factor Xa inhibitor. In some examples, the first layer comprises the anti-proliferative agent and the direct factor Xa inhibitor and the second layer comprises the direct factor IIa inhibitor. In some examples, the first layer comprises the direct factor IIa inhibitor and the direct factor Xa inhibitor and the second layer comprises the anti-proliferative agent. In some examples, the first layer comprises apixaban and argatroban and the second layer comprises sirolimus. In some examples, the therapeutic composition further comprises a top layer or coat of the same or different material as the first layer or the second layer.

In some examples, the coating further comprises a third layer. In some examples, the first layer comprises the direct factor IIa inhibitor, the second layer comprises the direct factor Xa inhibitor, and the third layer comprises the anti-proliferative agent. In some examples, the therapeutic composition further comprises a top layer or coat of the same or different material as the first layer, the second layer, or the third layer.

In some examples, the therapeutic composition comprises a coating disposed on at least one surface of the structure, and the coating further comprises a biodegradable polymer carrier. In some examples, the weight compositional ratio of the biodegradable polymer carrier to the two or more active substances is about 1:5 to about 3:2. In some examples, the therapeutic composition comprises a coating disposed on the external surface of the structure, and the coating comprises at least one layer of a polymeric material holding one or more of the direct factor IIa inhibitor and the direct factor Xa inhibitor.

In some examples, the therapeutic composition comprises a coating disposed on at least one surface of the structure, and the coating consists of a single layer of a polymeric material which releasably holds each of the direct factor IIa inhibitor and the direct factor Xa inhibitor. In some examples, the therapeutic composition further comprises a top layer or coat comprising the same or different polymeric material. In some examples, the direct factor IIa inhibitor and the direct factor Xa inhibitor are uniformly distributed in the polymeric material. In some examples, the direct factor IIa inhibitor and the direct factor Xa inhibitor are non-uniformly distributed in the polymeric material.

In some examples, the therapeutic composition comprises a coating disposed on at least one surface of the structure, and the coating comprises at least one layer of a polymeric material holding one or more of the direct factor IIa inhibitor, the direct factor Xa inhibitor, and the anti-proliferative agent. In some examples, the therapeutic composition comprises a coating disposed on at least one surface of the structure, and the coating consists of a single layer of a polymeric material which releasably holds each of the direct factor IIa inhibitor, the direct factor Xa inhibitor, and the anti-proliferative agent. In some examples, the therapeutic composition further comprises a top layer or coat comprising the same or different polymeric material. In some examples, the direct factor IIa inhibitor, the direct factor Xa inhibitor, and the anti-proliferative agent are uniformly distributed in the polymeric material. In some examples, the direct factor IIa inhibitor, the direct factor Xa inhibitor, and the anti-proliferative agent are non-uniformly distributed in the polymeric material.

In some examples, the two or more active substances are present in the polymeric material at weight ratios of about 4:1:3:1; about 5:3:2:1; about 4:2:2:1; about 5:2:3:1; about 6:3:3:1; about 10:5:5:1; or about 12:6:6:1 of a calcium chelating agent, direct factor IIa inhibitor to direct factor Xa inhibitor to anti-proliferative agent.

In another aspect, a method of treating one or more of inflammation, cell proliferation, smooth muscle cell proliferation, or clotting in a patient may comprise providing a structure having an external surface: deploying the structure at a target location in the patient's body so as to cause an injury at the location; and releasing from at least one surface of the deployed structure to the location of injury in the patient's body therapeutically effective amounts of a therapeutic composition including at least a calcium chelating agent, a direct factor IIa inhibitor, a direct factor Xa inhibitor, and an anti-proliferative agent.

In other examples, the therapeutic composition comprising a calcium chelating agent, a direct factor IIa inhibitor and a direct factor Xa inhibitor and an anti-proliferative is formulated to release said agents at a rate sufficient to inhibit one or more of inflammation, smooth muscle cell proliferation, cell proliferation, thrombin formation, fibrin formation, or clot formation, within about 3 hours to about 28 days or longer, or within about 3 hours to about 3 months or longer.

In another example, the therapeutic composition is formulated to release the two or more active substances, wherein the two or more substances comprise a calcium chelating agent, a direct IIa inhibitor, a direct Xa inhibitor, and an antiproliferative, to an injury site in a body lumen. In another example, the therapeutic composition is formulated to release the two or more active substances, wherein the two or more substances include of a calcium chelating agent, a direct IIa inhibitor, a direct Xa inhibitor, and an antiproliferative, to an injury site in a body lumen. In another example, the therapeutic composition is formulated to release the two or more active substances, wherein the two or more substances include of a calcium chelating agent, a direct IIa inhibitor, and an antiproliferative, to an injury site in a body lumen. In another example, the therapeutic composition is formulated to release the two or more active substances, wherein the two or more substances include of a calcium chelating agent, a direct IIa inhibitor, a direct Xa inhibitor, to an injury site in a body lumen. In another example, the therapeutic composition is formulated to release the two or more active substances, wherein the two or more substances include of a calcium chelating agent, a direct Xa inhibitor and an anti-proliferative, to an injury site in a body lumen.

In some examples, include of a calcium chelating agent, the direct factor IIa inhibitor comprises argatroban, the direct factor Xa inhibitor comprises apixaban, and the anti-proliferative agent comprises sirolimus.

In some examples, include of a calcium chelating agent, the direct factor IIa inhibitor comprises argatroban, the direct factor Xa inhibitor comprises Rivaroxaban, and the anti-proliferative agent comprises sirolimus.

In some examples, the therapeutic composition comprises a coating on the external surface of the structure or at least one surface of the structure and releasing the therapeutic composition comprises releasing the therapeutic composition from the coating. In some examples, the coating comprises one or more layers. In some examples, the coating comprises a biodegradable porous polymeric material, a degradable polymeric material, or a non-degradable polymeric material. In some examples, a calcium chelating agent, the direct factor IIa inhibitor and the direct factor Xa inhibitor are released faster than the anti-proliferative agent. In some examples, the direct factor IIa inhibitor and the direct factor Xa inhibitor enhance an anti-proliferative effect of the anti-proliferative agent. In some examples, the therapeutic composition is disposed within a drug reservoir fluidly coupled to the external surface of the structure and releasing the therapeutic composition comprises delivering the therapeutic from the drug reservoir to the external surface of the deployed structure.

In some examples, the injury is at least partially caused before deployment of the structure. In some examples, deployment of the structure causes the injury and the therapeutic composition is formulated to release a calcium chelating agent, the direct factor IIa inhibitor, the direct factor Xa inhibitor, or the anti-proliferative agent before the injury occurs.

In some examples, the therapeutic composition comprises a first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers may be the same or different.

In another example of any of the examples in this application, a therapeutic composition comprising two or more active substances on at least one surface of the device is configured to be positioned adjacent to an injury site in the patient's body, wherein adjacent to comprises one or more of the following: next to, touching, deployed at, expanded at, pushing against, placed against, or other. In a preferred example, the active substances are a calcium chelating agent, a direct factor IIa inhibitor and a direct factor Xa inhibitor. In another example the active substances are a calcium chelating agent, a direct factor IIa inhibitor, a direct factor Xa inhibitor and an anti-proliferative. In yet another example, the active substances are one of Argatroban. Rivaroxaban or Apixaban, and Sirolimus or Sirolimus analogue.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

These and other embodiments are described in further detail in the following description related to the appended drawing figures.

In still other instances, the therapeutic compositions of the present invention may be formulated to commence release of a calcium chelating agent and the direct factor IIa inhibitor before commencing release of and the direct factor Xa inhibitor. For example, the release of a calcium chelating agent may commence from 1 minute to 3 days, usually 3 hours to 1 day, after release of the direct factor IIa inhibitor has commenced.

In still other instances, the therapeutic compositions of the present invention may be formulated to commence release of a calcium chelating agent before commencing release of the direct factor IIa inhibitor and the direct factor Xa inhibitor. For example, the release of the of the direct factor Xa inhibitor may commence from 1 minute to 3 days, usually 3 hours to 1 day, after release of the direct factor IIa inhibitor and the direct factor IIa inhibitor has commenced.

In still other instances, the therapeutic compositions of the present invention may be formulated to commence release of the direct factor Xa inhibitor before commencing release of the direct factor IIa inhibitor. For example, release of the of the direct factor IIa inhibitor masy commence from 1 minute to 3 days, usually 3 hours to 1 day, after release of the direct factor Xa inhibitor has commenced.

Exemplary direct factor IIa inhibitors suitable for incorporation into the therapeutic compositions of the present invention include at least one of argatroban, dabigatran, ximelagatran, melagatran, efegatran, inogatran, atecegatran metoxil (AZD-0837), hirudin, hirudin analogs, bivalirudin, desirudin, and lepirudin. Presently preferred is argatroban, or a salt, isomer, solvate, analog, derivative, metabolite, or prodrugs thereof.

Exemplary direct factor Xa inhibitors suitable for incorporation into the therapeutic compositions of the present invention include at least one of apixaban, betrixaban, edoxaban, otamixaban, razaxaban, rivaroxaban, (r)-n-(2-(4-(1-methylpiperidin-4-yl) piperazin-1-yl)-2-oxo-1-phenylethyl)-1h-indole-6-carboxamide (LY-517717), daraxaban (YM-150), 2-1 (7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD (348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Presently preferred are rivaroxaban and apixaban, or a salt, isomer, solvate, analog, derivative, metabolite, or prodrugs thereof.

Exemplary anti-proliferative agents suitable for incorporation into the therapeutic compositions of the present invention include at least m-tor inhibitors selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. Preferred m-tor inhibitors include sirolimus, or a salt, isomer, solvate, analog, derivative, metabolite, or prodrugs thereof.

Exemplary anti-proliferative agents suitable for incorporation into the therapeutic compositions of the present invention also include paclitaxel, or a salt, isomer, solvate, analog, derivative, metabolite, or prodrug thereof, as well as antiplatelet drugs.

Preferred combinations of active agent pairs include a calcium chelating agent, argatroban as the direct factor IIa inhibitor and apixaban or rivaroxaban as the direct factor Xa inhibitor comprises.

In specific instances, the structure may comprise a scaffold having at least an outer surface, an inner surface, and one or more edge surfaces between the outer and inner surface. In such instances at least a portion of the outer surface may coated with the therapeutic composition, at least a portion of the inner surface may be coated with the therapeutic composition, at least a portion of the edge surfaces is coated with the therapeutic composition, and frequently two or three of such surfaces will be coated.

In specific instances, at least some of the surfaces, including the outer, inner, and edge surfaces, may have receptacles formed therein, and at least some of these receptacles may have therapeutic agent(s) therein. The receptacles may comprise one or more of wells, channels, holes, surface texture, and the like.

In specific instances, the therapeutic compositions may further comprise an excipient, an adjuvant, a polymeric carrier, or the like.

In specific instances, the three or more active substances may be mixed uniformly with each other. Alternatively or additionally, the three or more active substances may be layered separately from each other. Each layer may comprise an excipient mixed with the therapeutic agent, where the excipient(s) in two or more layers may the same or may be different in at least two of the three layers.

In specific instances, the, the devices may further comprise a control-release layer formed over the at least two, three or more active substances.

In specific instances, the therapeutic composition may include a base layer formed over a surface of the structure and a top layer formed over the base layer. The base layer and top layer may differ in at least some properties. For example, the base layer and top layer differ in at least one of drug dose, drug release rate, and drug release duration.

In preferred examples, the top layer of the therapeutic composition will be formulated to commence release of the active substances before commencing of the active substances from the base layer. For example, the active substances may be released from the top layer over a time period in the range from 1 hour to 7 days after the surface of the structure is positioned adjacent the injury site and/or the active substances may be released from the base layer over a time period in the range from 7 days to 12 months after the active substances have been substantially completely released from the top layer. For example, each of the base and top layers may comprise the at least three active substances are mixed in a biodegradable polymeric matrix.

In another aspect, the present invention provides a method for treating tissue injury in patients. The method comprises deploying a structure at a target tissue injury location in the patient's body lumen. A therapeutic composition is released from the deployed structure to the location of injury, where the therapeutic composition comprises at least a calcium chelating agent, a direct factor IIa inhibitor, a direct factor Xa inhibitor, and optionally an anti-proliferative agent.

The therapeutic composition may be positioned on an external surface of the device, on an internal surface of the device, or on both external and internal surfaces of the device.

The tissue injury may be caused by deploying the structure at the location or may preexists deploying the structure at the location.

In specific instances, the body lumen comprises a blood vessel and the therapeutic composition is formulated to locally release the at least two, three or more active substances to the injury site at a rate or a concentration sufficient to begin to inhibit or resolve one or more of inflammation, cell proliferation, internal elastic lamina (IEL) injury, fibrin formation, platelet aggregation, platelet activation, and clot formation or dissolution within about 3 hours to about 7 days after the structure is deployed.

In specific instances, the at least two, three or more active substances may be released to the injury site at a rate or a concentration sufficient to inhibit one or more of inflammation, cell proliferation, internal elastic lamina (IEL) injury, fibrin formation, platelet aggregation, platelet activation, and clot formation or dissolution for a period of at least 1 day, for a period of at least one week, for a period of at least one month, for a period of at least three months, for a period of at least six months, or for a period of at least one year after the surface of the structure is positioned adjacent the injury site.

In specific instances, the two, three or more active substances are released substantially simultaneously.

Alternatively or additionally, the direct factor IIa inhibitor and the direct factor Xa inhibitor are released substantially simultaneously and the anti-proliferative is released after the release of the direct factor IIa inhibitor and the direct factor Xa inhibitor has commenced. For example, the release of the of the anti-proliferative agent may commence in a period of 1 minute to 3 days, usually 6 hours to 1 day, after release of the direct factor IIa inhibitor and the direct factor Xa inhibitor has commenced.

Alternatively or additionally, the therapeutic composition may commences release of the direct factor IIa inhibitor before commencing release of the direct factor Xa inhibitor. For example, the release of the of the direct factor Xa inhibitor commences from 1 minute to 3 days, usually 6 hours to 1 day, after release of the direct factor IIa inhibitor has commenced.

Alternatively or additionally, the therapeutic composition may commence release of the direct factor Xa inhibitor before commencing release of the direct factor IIa inhibitor. For example, wherein the release of the of the direct factor IIa inhibitor commences from 1 minute to 3 days, usually 6 hours to 1 day, after release of the direct factor Xa inhibitor has commenced.

In some instances, the direct factor IIa inhibitor comprises at least one of argatroban, dabigatran, ximelagatran, melagatran, efegatran, inogatran, atecegatran metoxil (AZD-0837), hirudin, hirudin analogs, bivalirudin, desirudin, and lepirudin. In presently preferred instances, the direct factor IIa inhibitor comprises argatroban, or a salt, isomer, solvate, analog, derivative, metabolite, or prodrugs thereof.

In some instances, the direct factor Xa inhibitor comprises at least one of apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD (348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). In a presently preferred example, the direct factor Xa inhibitor comprises rivaroxaban, or a salt, isomer, solvate, analog, derivative, metabolite, or prodrugs thereof. In a second preferred example, the direct factor Xa inhibitor comprises apixaban, or a salt, isomer, solvate, analog, derivative, metabolite, or prodrugs thereof.

In some instances, the anti-proliferative agent comprises an m-Tor inhibitormay be selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof. A presently preferred anti-proliferative agent comprises sirolimus, or a salt, isomer, solvate, analog, derivative, metabolite, or prodrugs thereof.

In other instances, the anti-proliferative agent may comprise paclitaxel, or a salts, isomer, solvate, analog, derivative, metabolite, or prodrug thereof.

In still other instances, the anti-proliferative agent may comprise an antiplatelet drug.

Preferred combinations and pairings of active substances in the methods herein, comprise: a calcium chelating agent (1), the direct factor IIa inhibitor comprising (2) argatroban and the direct factor Xa inhibitor comprising apixaban and (3) the direct factor IIa inhibitor comprising argatroban, the direct factor Xa inhibitor comprises apixaban, and the anti-proliferative agent comprising sirolimus.

In some instances, the structure comprises a scaffold having at least an outer surface, an inner surface, and one or more edge surfaces between the outer and inner surfaces and wherein deploying comprises expanding the scaffold in the body lumen. Typically, at least a portion of the outer surface is coated with the therapeutic composition. Optionally, at least a portion of the inner surface is coated with the therapeutic composition. Further optionally, a portion of the edge surfaces is coated with the therapeutic composition.

In some instances, at least some of the surfaces may have receptacles formed therein and at least some of these receptacles have therapeutic agent therein wherein the receptacles may comprise one or more of wells, channels, holes, and surface texture.

In some instances, the release rate(s) of the active substances will be controlled. For example, excipients with different degradation or release rates can be added to different layers and/or combined with different active substances. Additionally or alternatively, a control-release layer may be formed over therapeutic composition to control the release of the three or more active substances.

In some, the therapeutic composition may include a base layer formed over the surface and a top layer formed over the base layer, wherein the base layer and top layer differ in at least some properties. In such instances, the therapeutic composition may be formulated to release the active substances substantially completely from the top layer before releasing the active substances from the base layer. For example, the active substances may be released from the top layer over a time period in the range from 1 hour to 7 days after the surface of the structure is positioned adjacent the injury site, and the active substances are released from the base layer over a time period in the range from 7 days to 12 months after the after the active substances have been substantially completely released from the top layer. In some instances, each of the base and top layers comprises the at least three active substances are mixed in a biodegradable polymeric matrix.

Often, the injury is at least partially caused before deployment of the structure, but more commonly deployment of the structure, e.g, stent expansion in an artery, causes the injury and wherein the therapeutic composition is formulated to release the calcium chelating agent EDTA the direct factor IIa inhibitor, the direct factor Xa inhibitor, or the anti-proliferative agent before, during, and/or following the injury occurs. Specific examples include vascular wall injury during vascular interventions, including, angioplasty, atherectomy, stent placement, graft placement, and the like.

In other instances, injury occurs during placement, implantation, or other introduction of a temporary or non-temporary device which is selected from the group consisting of access devices, infusion devices, tools, surgical instruments and tools, implants, bodily implants, hip implants, shoulder implants, knee implants, organ implants, luminal implants, vascular implants, stent-delivery systems, stents, stent-grafts, catheters, balloons, graft implants, grafts, aneurysm coils, valves, valve implants, shunts, left atrial appendage implants, foramen implants, leads, closure devices, clips, wound-closure devices and implants, sutures, patches, injection devices, needles inserted in the body, and needles inserted from outside the body.

Each class and type of anti-coagulation agent described and claimed herein, including but not limited to the inhibitors of the clotting cascade, such as the specific factor IIa inhibitor, factor Xa inhibitor, factor XI inhibitor, and factor XIa inhibitor listed herein, as well as the specific chelating agents and the specific anti-coagulation enhancers, may be combined one or more of the other classes and types of anti-coagulation agents described and claimed herein. Moreover, each class and type of anti-coagulation agent described and claimed herein may be formulated for any release profile as described and claimed such as bolus release, dual bolus release, and extended release.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a plot of HAoSMC cell proliferation in the presence of rapamycin and varying concentrations of Apixaban, in accordance with examples:

FIG. 1B shows a plot of HAoSMC cell proliferation in the presence of rapamycin and varying concentrations of Argatroban, in accordance with examples:

FIG. 1C shows a plot of HAoSMC cell proliferation in the presence of rapamycin and varying concentrations of Apixaban and Argatroban, in accordance with examples:

FIG. 1D shows a plot of HAoSMC cell proliferation in the presence of difference concentrations of Apixaban, in accordance with examples:

FIG. 1E shows a plot of HAoSMC cell proliferation in the presence of difference concentrations of Argatroban, in accordance with examples:

FIG. 2A shows a plot of activated clotting time (ACT) versus drug concentration, in accordance with examples:

FIG. 2B shows a plot of activated clotting time (ACT) versus drug concentration, in accordance with examples:

FIG. 2C shows a plot of activated clotting time (ACT) versus drug concentration, showing the synergistic effects of Apixaban in combination with Argatroban, in accordance with examples; and

FIG. 2D shows a plot of various synergistic effects of drug combination ratios between Apixaban and Argatroban, in accordance with examples.

### DETAILED DESCRIPTION OF THE DISCLOSURE

In the following detailed description, reference is made to the accompanying figures, which form a part hereof. In the figures, similar symbols typically identify similar components, unless context dictates otherwise. The illustrative examples described in the detailed description, figures, and claims are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain examples and examples are disclosed below, inventive subject matter extends beyond the specifically disclosed examples to other alternative examples and/or uses, and to modifications and equivalents thereof. Thus, the scope of the claims appended hereto is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples, however, the order of description should not be construed to imply that these operations are order dependent. Additionally, the structures, systems, and/or devices described herein may be embodied as integrated components or as separate components.

For purposes of comparing various examples, certain aspects and advantages of these examples are described. Not necessarily all such aspects or advantages are achieved by any particular example or embodiment. Thus, for example, various examples may be carried out in a manner that achieves or optimizes one advantage or group of advantages as taught herein without necessarily achieving other aspects or advantages as may also be taught or suggested herein.

Every example of the present invention may optionally be combined with any one or more of the other examples described herein. Every patent literature, and every non-patent literature, cited herein is incorporated herein by reference in its entirety.

The present invention disclosure is described in relation to drug-coated stents, drug-coated balloons, balloon reservoirs, heart implants, hip implants, knee implants, shoulder implants, and the like. However, one of skill in the art will appreciate that this is not intended to be limiting and the devices and methods disclosed herein may be used in other anatomical areas and in other surgical procedures or in other devices whether used as temporary devices or permanent devices.

As used herein, the term coagulation comprises one or more of thrombin formation, fibrin formation, platelet activation, platelet aggregation, and/or thrombus/clot formation. Coagulation typically arises in response to a body part injury and/or to a foreign body such as a device. This may lead to one or more of inflammation, injury, blockage of a lumen or vessel partially or fully, degradation of the device function, formation of clot, and/or adverse clinical events. In some examples, any of the devices described herein may, at least partially, cause an injury to the tissue which may initiate the coagulation cascade.

As used herein, the term anti-coagulant refers to an agent that inhibits one or more of thrombin formation, fibrin formation, platelet activation (typically indirectly), platelet aggregation (typically indirectly), thrombus (clot) formation, thrombin dissolution, fibrin dissolution, or thrombus dissolution, thereby inhibiting one or more of blockage of a lumen or vessel partially or fully, degradation of the device function, formation of clot, and/or adverse clinical events.

Inhibiting one or more of thrombin formation, fibrin formation, platelet activation, and/or platelet aggregation enables the inhibition of one or more of blockage of a lumen or vessel partially or fully, degradation of the device function, formation of thrombus (clot) formation, inflammation, and/or adverse clinical events.

Described herein are devices and methods for locally delivering a therapeutic composition to a patient. The therapeutic composition includes one or more agents which inhibit one or more of thrombin, fibrin, and/or thrombus formation or promote one or more of thrombin, fibrin, and/or thrombus dissolution. In preferred examples, the therapeutic composition includes one or more of a calcium chelating agent, a direct Xa inhibitor and a direct IIa inhibitor. In another preferred example, an anti-proliferative agent may be added to the therapeutic composition of the direct Xa inhibitor and/or the direct IIa inhibitor.

In some examples, the device or the implant comprising: a body structure having a surface configured to be implanted in a patient's body; and a therapeutic composition present on a surface of the body structure, therapeutic composition comprising at least one drug selected from the group consisting of a chelating agent, a direct factor IIa inhibitor, an a direct factor Xa inhibitor, wherein the therapeutic composition is formulated for a delayed release into an environment surrounding the body structure upon implantation of the body structure into environment.

In some examples, the implantable scaffold with the therapeutic composition is formulated for a rapid release into the environment surrounding the body structure a preselected time period after implantation of the body structure into environment.

In some examples, the implantable scaffold with the body structure comprises an expandable scaffold, an orthopedic implant or any type of therapeutic, diagnostic, or other structure intended for implantation in the patient's body, typically being an expandable scaffold, such as a vascular stent, a prosthetic heart valve, a patent foramen ovale (PFO) occlusion device, an atrial septal defect (ASD) occlusion device, a left atrial appendage (LAA) occlusion device, or similar expandable structure. Orthopedic implants include artificial joints, spinal implants, medullary screws, and the like.

In some examples, the implantable scaffold with the therapeutic composition is present at least partly on the surface of the body structure.

In some examples, the implantable scaffold with the therapeutic composition is present at least partly within a cavity or reservoir within the body structure.

In some examples, the implantable scaffold with the therapeutic composition is formulated to inhibit release of the at least one drug into the environment surrounding the body structure for a time period in a range from 5 minutes to 240 minutes, preferably from 5 minutes to 180 minutes, and more preferably from 5 minutes to 60) minutes.

In some examples, the implantable scaffold with the therapeutic composition is formulated to release at least 50% by weight, preferably at least 75% by weight of the at least one drug into the environment surrounding the body structure within 72 hours of implantation, preferably within 24 hours of implantation, more preferably within 6 hours of implantation, and even more preferably within 4 hour of implantation.

In some examples, the implantable scaffold with the therapeutic composition is formulated to release additional amounts of the at least one drug into the environment for a period of at least 3 days, preferably at least 7 days, more preferably 21 days, still more preferably at least 28 days, even more preferably at least 3 months, and often 6 months or more after implantation.

In some examples, the implantable scaffold with the drug comprises at least a chelating agent in the therapeutic composition is formulated to deplete calcium in the environment surrounding the body structure upon implantation of the body structure in environment.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy) ethane-N,N,N′,N′-tetraacetic acid (BAPTA), the surfactant-EDTA complex, quaternary ammonium EDTA salt, benzalkonium EDTA salt, EDTA complex, salts, analogue, solvate, hydrate and derivatives thereof.

In some examples, the implantable scaffold with the cationic anti-coagulation enhancer is selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a poly betaaminoester (PBAE), Histone, ethylenediamine, methylenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the at least one drug comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with the at least one drug comprises a direct factor Xa inhibitor selected from the group consisting of apixaban, betrixaban, edoxaban, otamixaban, razaxaban, rivaroxaban, (r)-n-(2-(4-(1-methylpiperidin-4-yl) piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-penty: 1]-3-oxo-3, 4-dihydro-quinoxaline-6-carboxylic acid (PD0313052).

In some examples, the implantable scaffold of claim 64, wherein the direct factor Xa inhibitor comprises apixaban, or a salt, isomer, solvate, analog, derivative, metabolite, or prodrugs thereof.

In some examples, the implantable scaffold of claim 64, wherein the direct factor Xa inhibitor comprises rivaroxaban, or a salt, isomer, solvate, analog, derivative, metabolite, or prodrugs thereof.

In some examples, the implantable scaffold with therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold of claim 14, wherein the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with the therapeutic composition further comprises an antiplatelet drug.

In some examples, the implantable scaffold with the therapeutic composition further comprises an antiproliferative agent selected from the group consisting of mycophenolate mofetil, mycophenolate sodium, azathioprine.

In some examples, the implantable scaffold with the implantable scaffold has at least an outer surface, an inner surface, and one or more edge surfaces between the outer and inner surfaces.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold at least some of the surfaces have receptacles formed therein and at least some of receptacles have therapeutic agent therein.

In some examples, the implantable scaffold with the receptacles comprise one or more of wells, channels, holes, and surface texture.

In some examples, a therapeutic composition present on a surface of the scaffold. The therapeutic composition comprising at least one chelating agent. In some examples, the implantable scaffold the therapeutic composition is present at least partly on the surface of scaffold structure. In some examples, the implantable scaffold the therapeutic composition is present at least partly within a cavity or reservoir within the scaffold structure.

In some examples, the implantable scaffold with the chelating agent in the therapeutic composition is formulated to deplete calcium in the environment surrounding the scaffold upon implantation of the scaffold structure in environment.

In some examples, the implantable scaffold with the therapeutic composition is formulated to release at least 50%, preferably at least 75%, by weight of the at least one chelating agent into the vascular environment within 72 hours of implantation, preferably within 24 hours of implantation, more preferably within 6 hours of implantation, and even more preferably within 4 hours of implantation.

In some examples, the implantable scaffold with the therapeutic composition is formulated to release additional amounts of the at least one chelating agent into the environment for a period of at least 3 days, preferably at least 7 days, more preferably 21 days, still more preferably at least 28 days, even more preferably at least 3 months, and often 6 months or more after implantation.

In some examples, the implantable scaffold with the therapeutic composition consists essentially of chelating agent.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA). 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA). 1,2-bis(2-aminophenoxy) ethane-N,N,N′,N′-tetraacetic acid (BAPTA), the surfactant-EDTA complex, quaternary ammonium EDTA salt, benzalkonium EDTA salt. EDTA complex, salts, analogue, solvate, hydrate and derivatives thereof.

In some examples, the implantable scaffold with the cationic anti-coagulation enhancers is selected from a group consisting of cationic polymer or compounds including but not limit to poly(L-lysine) (PLL), linear polyethyleneimine (PEI), branch polyethyleneimine (PEI), chitosan. PAMAM dendrimers, and poly(2-dimethylamino)ethyl methacrylate (pDMAEMA), protamine, polylysine, a polybetaaminoester (PBAE). Histone, ethylenediamine, methylenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the chelating agent is present in the therapeutic composition at a weight percent from 10% to 100%.

In some examples, the implantable scaffold with therapeutic composition comprises additional active and/or inactive substances.

In some examples, the implantable scaffold with the additional active and/or inactive substances are present in the therapeutic composition at a weight percent from 20% to 90%.

In some examples, the implantable scaffold with the therapeutic composition further comprises at least one anti-coagulant.

In some examples, the implantable scaffold with the therapeutic composition is formulated to release at least one anti-coagulant at a rate equal to that of the chelating agent

In some examples, the implantable scaffold with the therapeutic composition is formulated to release at least one anti-coagulant at a rate slower than that of the chelating agent.

In some examples, the implantable scaffold with the therapeutic composition is formulated to release at least one anti-coagulant at a rate faster than that of the chelating agent.

In some examples, the implantable scaffold with the at least one anti-coagulant is selected from the group consisting of a direct factor IIa inhibitor and a direct factor Xa inhibitor.

In some examples, the implantable scaffold with the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with any one of the at least one anti-coagulant comprises adirect factor Xa inhibitor selected from the group consisting of apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150). 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-penty: 1]-3-oxo-3, 4-dihydro-quinoxaline-6-carboxylic acid (PD0313052).

In some examples, the implantable scaffold with the direct factor Xa inhibitor comprises apixaban, or a salt, isomer, solvate, analog, derivative, metabolite, or prodrugs thereof.

In some examples, the implantable scaffold with the direct factor Xa inhibitor comprises rivaroxaban, or a salt, isomer, solvate, analog, derivative, metabolite, or prodrugs thereof.

In some examples, the implantable scaffold with the therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold with the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with therapeutic composition further comprises an antiplatelet drug.

In some examples, the implantable scaffold with therapeutic composition further comprises an antiproliferative agent selected from the group consisting of mycophenolate mofetil, mycophenolate sodium, azathioprine.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold with at least some of the surfaces have receptacles formed therein and at least some of receptacles have therapeutic agent therein.

In some examples, the implantable scaffold with the receptacles comprises one or more of wells, channels, holes, and surface texture.

In some examples, a method for treating a vascular tissue injury in a patient, the method comprising: implanting a scaffold structure at a target location in the patient's vasculature proximate the tissue injury; and releasing a drug composition including at least one chelating agent from the implanted scaffold structure into the vasculature, wherein the chelating agent is released sufficiently rapidly into the vasculature to prevent blood clotting and inhibit the fibrin formation.

In some examples, the method with at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In some examples, the implantable scaffold with the therapeutic composition consists essentially of chelating agent.

In some examples, the implantable scaffold with the chelating agent is selected from a group consisting of ethylenediaminetetraacetic acid (EDTA), calcium disodium edetate, magnesium dipotassium edetate, magnesium disodium edetate, disodium edetate, tetrasodium edetate, trisodium edetate, monoammonium EDTA salt, diammonium EDTA salt, triammonium EDTA salt, benzyldimethyltetradecylammonium EDTA salt, tridodecy lmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTPA), hydroxyethylethylenediaminetriacetic acid (HEEDTA), diaminocyclohexanetetraacetic acid (CDTA), 1,2-bis(2-aminophenoxy) ethane-N,N,N′,N′-tetraacetic acid (BAPTA), the surfactant-EDTA complex, quaternary ammonium EDTA salt, benzalkonium EDTA salt, EDTA complex, salts, analogue, solvate, hydrate and derivatives thereof.

In some examples, the implantable scaffold with the cationic anti-coagulation enhancer is selected from a group consisting of cationic polymer or compounds including but not limit to poly(L-lysine) (PLL), linear polyethyleneimine (PEI), branch polyethyleneimine (PEI), chitosan, PAMAM dendrimers, and poly(2-dimethylamino)ethyl methacrylate (pDMAEMA), protamine, polylysine, a poly betaaminoester (PBAE), Histone, ethylenediamine, methy lenediamine, ammonium chloride, melamine, histamine, histidine, analogue, solvate, hydrate and derivatives thereof.

In some examples, the implantable scaffold with the chelating agent consists essentially of ethylenediaminetetraacetic acid (EDTA).

In some examples, the implantable scaffold with the cationic anti-coagulation enhancer consists essentially of benzyldimethyltetradecylammonium chloride.

In some examples, the implantable scaffold with the cationic anti-coagulation enhancer consists essentially of linear polyethyleneimine (PEI).

In some examples, the implantable scaffold with the chelating agent is present in the therapeutic composition at a weight percent from 10% to 100%.

In some examples, the implantable scaffold with therapeutic composition comprises additional active and/or inactive substances.

In some examples, the implantable scaffold with the additional active and/or inactive substances are present in the therapeutic composition at a weight percent from 20% to 90%.

In some examples, the implantable scaffold with the therapeutic composition further comprises at least one anti-coagulant.

In some examples, the implantable scaffold with the therapeutic composition is formulated to release at least one anti-coagulant at a rate equal to that of the chelating agent

In some examples, the implantable scaffold with the therapeutic composition is formulated to release at least one anti-coagulant at a rate slower than that of the chelating agent.

In some examples, the implantable scaffold with the therapeutic composition is formulated to release at least one anti-coagulant at a rate faster than that of the chelating agent.

In some examples, the implantable scaffold with the at least one anti-coagulant is selected from the group consisting of a direct factor IIa inhibitor and a direct factor Xa inhibitor.

In some examples, the implantable scaffold with the at least one anti-coagulant comprises a direct factor IIa inhibitor selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

In some examples, the implantable scaffold with the at least one direct factor IIa inhibitor comprises argatroban, or a salt, isomer, solvate, analog, derivative, metabolite, or prodrugs thereof.

In some examples, the implantable scaffold with any one of the at least one anti-coagulant comprises a direct factor Xa inhibitor selected from the group consisting of apixaban, betrixaban, edoxaban, otamixaban, razaxaban, rivaroxaban, (r)-n-(2-(4-(1-methylpiperidin-4-yl) piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

In some examples, the implantable scaffold with the direct factor Xa inhibitor comprises apixaban, or a salt, isomer, solvate, analog, derivative, metabolite, or prodrugs thereof.

In some examples, the implantable scaffold with the direct factor Xa inhibitor comprises rivaroxaban, or a salt, isomer, solvate, analog, derivative, metabolite, or prodrugs thereof.

In some examples, the implantable scaffold with the therapeutic composition further comprises an mTOR inhibitor selected from a group consisting of sirolimus, biolimus, everolimus, myolimus, novolimus, ridaforolimus, temsirolimus, zotarolimus, or salts, isomers, solvates, analogs, derivatives, metabolites, or prodrugs thereof.

In some examples, the implantable scaffold with the mTOR inhibitor comprises sirolimus, or a salt, isomer, solvate, analog, derivative, metabolite, or prodrugs thereof.

In some examples, the implantable scaffold with therapeutic composition further comprises paclitaxel, or a salt, isomer, solvate, analog, derivative, metabolite, or prodrug thereof.

In some examples, the implantable scaffold with therapeutic composition further comprises an antiplatelet drug.

In some examples, the implantable scaffold with therapeutic composition further comprises an antiproliferative agent selected from the group consisting of mycophenolate mofetil, mycophenolate sodium, azathioprine.

In some examples, the implantable scaffold with at least a portion of the outer surface is coated with the therapeutic compositions.

In some examples, the implantable scaffold with at least a portion of the inner surface is coated with the therapeutic compositions.

In some examples, the implantable scaffold with a portion of the edge surfaces is coated with the therapeutic compositions.

In some examples, the implantable scaffold with at least some of the surfaces have receptacles formed therein and at least some of receptacles have therapeutic agent therein.

In some examples, the implantable scaffold with the receptacles comprises one or more of wells, channels, holes, and surface texture.

In some examples, a method for treating a vascular tissue injury in a patient, the method comprising: implanting a scaffold structure at a target location in the patient's vasculature proximate the tissue injury; and releasing a drug composition including at least one chelating agent from the implanted scaffold structure into the vasculature, wherein the chelating agent is released sufficiently rapidly into the vasculature to prevent blood clotting and inhibit the fibrin formation.

In some examples, the method with at least 75% by weight of the at least one chelating agent is released into the vasculature within 72 hours of implantation, preferably within 24 hours of implantation, more preferably within 6 hours of implantation, and even more preferably within 4 hour of implantation, and usually between 10 minutes and 4 hours.

In some examples, the method with the therapeutic composition is positioned on at least one of an internal surface and an external surface of the implantable scaffold.

In some examples, the method with the therapeutic composition is positioned on both an external and an internal surface of the implantable scaffold.

In some examples, the method with the tissue injury is caused by expanding the scaffold at the location.

In some examples, the method with the tissue injury pre-exists deploying the structure at the location.

In some examples, the devices described herein can be configured to release a factor Xa inhibiting agent to a mammalian body, lumen, tissue, and/or device surface prior to an injury to said tissue, concurrent with injury to said tissue, or after an initial injury to said tissue. The device is introduced into said mammalian body and advanced to said tissue site or body lumen. In some specific examples, the device is expanded against said tissue to release said agent. In other examples, the device is expanded against said tissue to perform a function such as opening up a vessel or lumen and to release said agent. In specific examples, the device is a stent or a balloon catheter. In yet another example, the device is placed adjacent to said tissue. In specific examples, the device releases said agent to a tissue segment adjacent to the device in the amount ranging from 0.01 ng/mg of tissue to 1000 ng/mg of tissue, preferably ranging from 0.1 ng/mg tissue to 500 ng/mg of tissue, more preferably ranging from 1 ng/mg of tissue to 150 ng/mg of tissue.

In some examples, the direct factor Xa inhibitor is selected from the group consisting of apixaban, betrixaban, edoxaban, otamixaban, razaxaban, rivaroxaban, (r)-n-(2-(4-(1-methylpiperidin-4-yl) piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl) pyrrolidin-3-yl)oxy) phenyl) propanoic acid hydrochloride pentahydrate (DX9065a), letaxaban (TAK-442), GW813893, JTV-803, KFA-144, DPC-423, RPR-209685, MCM-09, or antistasin. For example, the direct factor Xa inhibitor may comprise rivaroxaban, or a salt, isomer, solvate, analog, derivative, metabolite, or prodrugs thereof. Alternatively, or in combination, the direct factor Xa inhibitor may comprise apixaban.

In some examples, an IC50 of the direct factor Xa inhibitor is within a range of about 0.01 nM to about 1000 nM. For example, the IC50 of the direct factor Xa inhibitor is within a range of about 0.1 nM to about 1000 nM, about 1 nM to about 1000 nM, or about 10 nM to about 1000 nM.

In some examples, the therapeutically effective dose of the direct factor Xa inhibitor is within a range of about 1 microgram to 10 mg, more preferably is within a range of about 50) micrograms to about 10 mg. For example, the therapeutically effective dose of the direct factor Xa inhibitor may be within a range of about 0.1 mg to about 10 mg or about 1 mg to about 5 mg.

In some examples, the therapeutically effective dose is sufficient to generate a tissue concentration of the direct factor Xa inhibitor of about 0.001 ng/g tissue to about 100 mg/g tissue, preferably, the therapeutically effective dose is sufficient to generate a tissue concentration of the direct factor Xa inhibitor of about 0.01 ng/g tissue to about 100 mg/g tissue, more preferably, the therapeutically effective dose is sufficient to generate a tissue concentration of the direct factor Xa inhibitor of about 0.1 ng/g tissue to about 100 mg/g tissue. For example, the therapeutically effective dose may be sufficient to generate a tissue concentration of the direct factor Xa inhibitor of about 0.2 ng/g tissue to about 100 mg/g tissue, about 0.5 ng/g tissue to about 100 mg/g tissue, about 1 ng/g tissue to about 100 mg/g tissue, about 10 ng/g tissue to about 100 mg/g tissue, or about 100 ng/g tissue to about 100 mg/g tissue: in about 1 day, 30 days, 60 days, 90 days, or 120 days after introducing the therapeutically effective dose. In some examples, the therapeutically effective dose is sufficient to generate a blood concentration of the direct factor Xa inhibitor which is less than about 200 ng/ml, 100 ng/ml, 50 ng/ml 25 ng/ml, or 10) ng/ml. In some examples, the therapeutically effective dose is sufficient to generate a blood concentration of the direct factor Xa inhibitor which is less than a systemic therapeutic concentration of the direct factor Xa inhibitor for any systemic indication. In some examples, the therapeutically effective dose is sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory ophthalmic condition or disease. In some examples, the therapeutically effective dose is sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory ophthalmic condition or disease for more than about 6 hours to about 3 days.

In some examples, the therapeutically effective dose is sufficient to maintain a tissue concentration of the direct factor Xa inhibitor of about 0.1 ng/g tissue to about 100 mg/g tissue for about 1 day to about 120 days, for about 1 day to about 1 year, 30 days to about 1 year, 3 months to about 1 year, or 6 months to about 1 year. In some other examples, the device releases said agent prior to engaging (or coupling or contacting) of the device to the tissue site.

In some specific examples, the device locally releases said agent to a tissue segment in the amount ranging from about 10 ng/mg to 200 ng/mg within about 3 hours from tissue injury and/or release of the agent to the tissue segment. In a preferred example, the adjacent tissue segment drug (e.g., tissue 5 mm proximal and 5 mm distal to the tissue segment) concentration ranges from about 0.1 ng/mg of tissue to about 100 ng/mg of tissue, preferably ranges from about 1 ng/mg of tissue to 100 ng/mg of tissue, at about 3 hours from tissue injury and/or release of the agent to the tissue segment. In a preferred example, the tissue concentration in the tissue segment at 3 hours after injury and/or release of said agent to the tissue segment ranges from about 100,000 times the IC50 of factor Xa inhibition to 10,000,000 times the IC50 of factor Xa inhibition, preferably ranges from 500,000 times to 5,000,000 times the IC50 of factor Xa inhibition. The tissue concentration in the adjacent tissue segment (e.g., +5 mm) at 3 hours after release of said agent to the tissue segment ranges from 100 times the IC50 of factor Xa inhibition to 1,000,000 times the IC50 of factor Xa inhibition, preferably ranges from 1,000 times to 100,000 times the IC50 of factor Xa inhibition. In a preferred example, the tissue concentration in the tissue segment at about 24 hours after injury and/or release of said agent to the tissue segment ranges from 100,000 times the IC50 of factor Xa inhibition to 1000,000 times the IC50 of factor Xa inhibition, preferably ranges from 1000 times to 20,000 times the IC50 of factor Xa inhibition. In a preferred example, the agent is rivaroxaban, apixaban, and/or analogs, derivatives, or salts thereof. In a most preferred example, the agent is apixaban.

In another example, the combination of apixaban and argatroban have an additive effect on thrombin formation inhibition or dissolution.

In some examples, a combination of factor IIa inhibitor and factor Xa inhibitor are released from a device to a mammalian body, lumen, tissue, and/or device surface after injury at sufficient concentrations in the tissue segment and adjacent tissue segments within about 3 hours after injury to inhibit thrombus (clot) formation. In a preferred example, the agents are apixaban and argatroban.

In some examples, the combination of apixaban and argatroban released from a device containing an mTOR inhibitor such as sirolimus maintains or enhances the antiproliferative effect of said mTOR at the tissue segment site while inhibiting thrombus formation at the said tissue segment site.

In some examples, the combination of apixaban and argatroban released from a device containing an mTOR inhibitor inhibits thrombus formation on the device surface.

In some examples, the device is coated or loaded with one or more agents comprising apixaban, argatroban and an mTOR inhibitor. The coating coats one or more surfaces of the device, preferably coating all surfaces of the device including the abluminal and luminal surfaces of the device. Alternatively, or in combination, structural elements of the device are loaded with the one or more agents. In a specific example, the one or more agents are contained in a drug polymer matrix, or contained in a polymer top layer or coat, or is coated as a top layer or coat. In a preferred example of a device configured to release two or more agents, the agents are contained in the same polymer matrix or a different polymer matrix, or one agent is in a polymer matrix while the other agent is under a top polymer coat. In yet another preferred example, the device contains three agents in the same polymer matrix. In another example, each of the drugs is contained in a separate polymer matrix. In yet another example, two of the agents are contained in one polymer matrix while the third agent is contained in a separate polymer matrix or a top layer or coat. In yet another example, the one or more agents are contained in the same polymer matrix and a top layer or coat of a polymer material covers the surface of the device.

While many of the examples described herein depict one or more active substances being coated on a device for local delivery of the one or more active substances, it will be understood by one of ordinary skill in the art that any of the devices described herein may locally delivery one or more of the active substances through any other means. For examples, one or more of the active substances may be coated, dipped, printed, deposited, painted, brushed, loaded, or otherwise disposed on one or more surfaces of the device for local delivery. In some examples, one or more of the active substances may be incorporated into the backbone structure of the device. Alternatively, or in combination, one or more of the active substances may be locally delivered via a drug reservoir coupled to the device. In some examples, one or more or the active substances may be coated or otherwise disposed directly onto one or more surfaces of the device. In some examples, one or more or the active substances may be coated or otherwise disposed on one or more surfaces of the device in a carrier such as a polymer matrix. In some examples, one or more of the active substances may be cross-linked with a polymer, or to itself, or to another drug (in order to be another active substance, e.g., after the links are broken in vivo). In some examples, the carrier may be an excipient, a polymer, or other types of material to facilitate applying or controlling the drug onto the device or controlling release of the drug from the device or protecting the drug from washing out during entry or deployment into the body. In some examples, the carrier may comprise a microsphere or a nanosphere.

When the therapeutic composition comprises a calcium chelating agent, a direct factor IIa inhibitor, a direct factor Xa inhibitor, and an anti-proliferative agent, the therapeutic composition may be present in the carrier material at weight ratios of 1:3:1, 3:2:1, 2:2:1, 2:3:1, 3:3:1, 5:5:1, or 6:6:1, respectively. In some examples, the weight compositional ratio of the direct factor IIa inhibitor to the direct factor Xa inhibitor to the anti-proliferative agent in the therapeutic composition may be about 5:5:2. In some examples, the weight compositional ratio of the direct factor IIa inhibitor to the direct factor Xa inhibitor to the anti-proliferative agent in the coating may be within a range of about 6:6:1 to 1:3:1.

When the therapeutic composition comprises a calcium chelating agent, a direct factor IIa inhibitor, a direct factor Xa inhibitor, and an anti-proliferative agent, the release rate ratio of the direct factor IIa inhibitor to the direct factor Xa inhibitor to the anti-proliferative agent may be about 1:1:1 to about 4:4:1. In some examples, the coating may be configured to release the direct factor IIa inhibitor, the direct factor Xa inhibitor, and the anti-proliferative agent at the same rate. In other examples, the coating may be configured to release the direct factor IIa inhibitor, the direct factor Xa inhibitor, and the anti-proliferative agent at different rates.

In some examples, the coating may be configured to release the direct factor IIa inhibitor at a rate of about 4 μg/hour/mm to about 14 μg/day/mm per device.

In some examples, the coating may be configured to release the direct factor Xa inhibitor at a rate of about 4 μg/hour/mm to about 14 μg/day/mm per device.

In some examples, the coating may be configured to release the anti-proliferative agent at a rate of about 1 μg/hour/mm to about 4 μg/day/mm per device.

In some examples, the direct factor IIa inhibitor may have an inhibition potency for factor IIa ranging from about 0.001 nM to about 100 nM.

In some examples, the direct factor Xa inhibitor may have an inhibition potency for factor Xa ranging from about 0.001 nM to about 50 nM.

In some examples, the direct factor IIa inhibitor may comprise argatroban, or a salt, isomer, solvate, analog (including deuterated analog), derivative, metabolite, or prodrugs thereof.

In some examples, the direct factor Xa inhibitor may comprise apixaban, or a salt, isomer, solvate, analog (including deuterated analog), derivative, metabolite, or prodrugs thereof.

In some examples, the direct factor Xa inhibitor may comprise rivaroxaban, or a salt, isomer, solvate, analog (including deuterated analog), derivative, metabolite, or prodrugs thereof.

In some examples, the anti-proliferative agent may comprise rapamycin, or a salt, isomer, solvate, analog (including deuterated analog), derivative, metabolite, or prodrugs thereof.

In some examples, the direct factor Xa inhibitor may comprise apixaban, the direct factor IIa inhibitor may comprise argatroban, and the anti-proliferative agent may comprise rapamycin.

In some examples, the therapeutic composition is disposed on the external surface of the structure and on the internal surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the first layer or the second layer may comprise one or more bioactive agents and the other layer may not comprise a bioactive agent.

In some examples, the first layer may comprise one or more bioactive agents and the second layer may comprise one or more bioactive agents. The first layer and the second layer may be configured to release their respective bioactive agents at the same rate. Alternatively, the first layer and the second layer may be configured to release their respective bioactive agents at different rates.

In some examples, the first layer may comprise a calcium chelating agent, a direct factor IIa inhibitor and the second layer may comprise a direct factor Xa inhibitor. In some examples, the first layer may comprise a direct factor Xa inhibitor and the second layer may comprise a direct factor IIa inhibitor.

In some examples, the first layer may comprise an anti-proliferative agent and the second layer may comprise a calcium chelating agent, a direct factor IIa inhibitor and a direct factor Xa inhibitor.

In some examples, the first layer may comprise a calcium chelating agent, a direct factor IIa inhibitor and a direct factor Xa inhibitor and the second layer may comprise an anti-proliferative agent.

In some examples, one or both of the layers may comprise a polymeric material as described herein. For example, the first layer may comprise a polymeric material and the second layer may not comprise a polymeric material. Alternatively, the first layer may not comprise a polymeric material and the second layer may comprise a polymeric material. Alternatively, both the first layer and the second layer may comprise the same or a different polymeric material and/or polymeric material concentration and/or formulation.

The first layer and/or second layer may be disposed on an external surface of the stent (e.g., on the abluminal surface of one or more filaments of the stent), on an internal surface of the stent (e.g., on the luminal surface of one or more filaments of the stent), or on both the external surface and the internal surface of the stent (e.g., partially or fully surround one or more filaments of the stent). The first layer and the second layer of the coating are shown fully coating each of the filaments of the stent. However, it will be understood by one of ordinary skill in the art that first layer and the second layer may coat the stent differently. For example, the first layer of the coating may fully surround each of the filaments of the stent while the second layer may be applied only on one surface (e.g., luminal or abluminal) of the stent. In some examples, both layers are not on an external surface of the stent.

In some examples, the first layer, the second layer, and/or the third layer may comprise one or more bioactive agents and one or more of the other layers may not comprise a bioactive agent.

In some examples, the first layer may comprise one or more bioactive agents, the second layer may comprise one or more bioactive agents, and the third layer may comprise one or more bioactive agents. The first layer, the second layer, and the third layer may be configured to release their respective bioactive agents at the same rate. Alternatively, two or more of the first layer, the second layer, or the third layer may be configured to release their respective bioactive agents at different rates.

In some examples, the first layer may comprise a calcium chelating agent, a direct factor IIa inhibitor, the second layer may comprise a direct factor Xa inhibitor, and the third layer may comprise an anti-proliferative agent. In some examples, the first layer may comprise a calcium chelating agent, the second layer may comprise a direct factor Xa/IIa inhibitor, and the third layer may comprise an anti-proliferative agent.

In some examples, the first layer may comprise an anti-proliferative agent, the second layer may comprise a calcium chelating agent, a direct factor IIa inhibitor, and a direct factor Xa inhibitor. In some examples, the first layer may comprise an anti-proliferative agent, the second layer may comprise a calcium chelating agent, and the third layer may comprise a direct factor IIa inhibitor and a direct factor IIa inhibitor.

In some examples, one, two, or three of the layers may comprise a polymeric material as described herein. For example, the first layer may comprise a polymeric material, the second layer may comprise a polymeric material, and the third layer may not comprise a polymeric material. The layers may comprise the same or a different polymeric material and/or polymeric material concentration and/or formulation.

The first layer, second layer, and/or third layer may be disposed on an external surface of the stent (e.g., on the abluminal surface of one or more filaments of the stent), on an internal surface of the stent (e.g., on the luminal surface of one or more filaments of the stent), or on both the external surface and the internal surface of the stent (e.g., partially or fully surround one or more filaments of the stent). The first layer, the second layer, and third layer of the coating are shown fully coating each of the filaments of the stent. However, it will be understood by one of ordinary skill in the art that one or more of the first layer, the second layer, and the third layer may coat the stent differently. For example, the first layer of the coating may fully surround each of the filaments of the stent while the second layer may be applied to a first surface (e.g., luminal) and the third layer may be applied to a second surface (e.g., abluminal) of the stent. In some examples, the layers are not on an external surface of the stent.

It will be understood by one of ordinary skill in the art based on the description herein that any of the coating described herein may comprise any number of layers desired and that the layers may comprise any number and combination of bioactive agents, carrier materials, etc. desired.

In some examples, the therapeutic coating may comprise a single layer comprising a plurality of bioactive agents. For example, the single layer may comprise a therapeutic composition of a calcium chelating agent, an anti-proliferative agent, a direct factor Xa inhibitor, and/or a direct factor IIa inhibitor as described herein. The bioactive agents may be uniformly distributed in a carrier material of the single layer. Alternatively, the bioactive agents may be non-uniformly distributed in a carrier material of the single layer. In some examples, the carrier material is a polymeric material.

In some examples, the therapeutic composition may comprise a calcium chelating agent, a direct factor IIa inhibitor, a direct factor Xa inhibitor, and/or an anti-proliferative agent as described herein.

In still further examples, a substantial amount, or substantially all, of each of the fibrin formation inhibition, thrombus formation-inhibiting or fibrin or thrombus dissolution-promoting agent(s) is released from the device within about 1 min, 15 min, 30 min, 1 hr, 6 hr, 12 hr, 1 day, 2 days, 3 days, 1 week, 2 weeks, 3 weeks, 1 month, 2 months, 3 months, 4 months, 5 months, 6 months, 9 months, or 1 years. In a preferred example, the one or more agents comprising a calcium chelating agent, a factor IIa inhibitor or factor Xa inhibitor are configured to substantially release over at least 28 days, preferably over at least 90 days, over at least 6 months, or over at least 1 year.

In some examples, the therapeutic coating may locally release the one or more cationic anti-coagulation enhancers to the device surface, which may be disposed adjacent a tissue segment of interest. The coating may release the one or more cationic anti-coagulation enhancers to the device surface in sufficient dose/concentrations to inhibit platelet aggregation, thrombus, thrombin, and/or clot formation. The vicinity to the wall or tissue contacting blood should have sufficient drug concentration to inhibit platelet aggregation, fibrin, thrombin, and/or clot formation.

In some examples, the therapeutic coating may locally release the one or more cationic anti-coagulation enhancers agents into the blood adjacent the tissue segment.

In some examples, the therapeutic coating may locally release the one or more bioactive agents to the device surface, which may be disposed adjacent a tissue segment of interest. The coating may release the one or more bioactive agents to the device surface in sufficient dose/concentrations to inhibit platelet aggregation, thrombus, thrombin, and/or clot formation. The vicinity to the wall or tissue contacting blood should have sufficient drug concentration to inhibit platelet aggregation, fibrin, thrombin, and/or clot formation.

In some examples, the therapeutic coating may locally release the one or more bioactive agents into the blood adjacent the tissue segment.

As used herein, the term coating refers to one or more layers disposed on a surface of a device. In some examples, a single coating is applied to the device. In other examples, one or more coatings are used. In some examples, each component of the therapeutic composition is disposed within the same layer of the coating. In some examples, at least one component of the therapeutic composition is disposed in a different layer of the coating. In some examples, one or more component of the therapeutic composition is coated on all surfaces of the device. In some examples, one or more component of the therapeutic composition is coated on a single surface of the device. In some examples, one or more component of the therapeutic composition is coated on two or more surfaces of the device. In some examples, one or more component of the therapeutic composition is coated on at least four surfaces (e.g, an inner surface, an outer surface, a first side, and a second side) of the device.

In addition to, the coatings described herein. For example, in some instances, the device can comprise one or more components of the therapeutic composition in a reservoir on or in the device. Alternatively, or in combination, the device can comprise one or more components of the therapeutic composition dispersed within the device structure.

In some examples, the therapeutic composition may be formulated to release one or more of the agents at a dose substantially below a systemic therapeutic dose of each agent to minimize off-target effects. Preferably, the dose is at least about 5 times lower than the systemic dose or more preferably about 10 times lower than the systemic dose.

In many examples, a tissue segment is composed of the tissue segment coupled to the device releasing agent. For example, if the stent is 20 mm in length, the tissue segment is 20 mm in length. In another specific example, the agent is released beyond the tissue segment. For example, when the tissue segment coupled to a device is 20 mm in length, the tissue adjacent to the tissue segment is called the adjacent tissue segment. In many cases the adjacent tissue segment ranges from 1 mm to 10 mm, preferably within a range from 1 mm to 5 mm, more preferably about 5 mm proximal and/or distal to the tissue segment, and most preferably is about 5 mm proximal and distal to the tissue segment.

As used herein, the term “coating” refers to a layer of polymer and/or drug (or therapeutic agent or active agent) disposed on a surface of a device structure. The layer may comprise a polymer, a drug, or a combination of a drug and a polymer.

As used herein, the term “top layer or coat” refers to an outer-most layer of a coating. The top layer or coat may comprise a polymer, a drug (or therapeutic agent or active agent), or a combination of a drug and a polymer. The top layer or coat may comprise the same polymer or a different polymer as layers of coating disposed there below. The top layer or coat may comprise the same drug or a different drug(s) as layers of coating disposed there below.

As used herein, the term “matrix” refers to a mixture of a drug (or therapeutic agent or active agent) and a polymer.

The terms anti-thrombin, thrombin inhibiter, and thrombin formation inhibitor are used interchangeably herein. Also, the terms anti-fibrin, fibrin inhibitor, and fibrin formation inhibitor are used interchangeably herein.

As used herein, a calcium chelating agent refer to any agent that could chelate calcium.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, apixaban, betrixaban, edoxaban, otamixaban, razaxaban, rivaroxaban. (r)-n-(2-(4-(1-methylpiperidin-4-yl) piperazin-1-yl)-2-oxo-1-phenylethyl)-1h-indole-6-carboxamide (LY-517717), daraxaban (YM-150), or 2-[(7-carbamimidoylnaphthalen-2-yl)methyl-[4-(1-ethanimidoylpiperidin-4-yl)oxyphenyl ]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Preferred direct Xa inhibitors include apixaban and rivaroxaban.

As used herein, a direct factor IIa inhibitor refers to a direct, selective inhibitor of factor Ila (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, argatroban, dabigatran, ximelagatran, melagatran, efegatran, inogatran, atecegatran metoxil (AZD-0) 837), hirudin, hirudin analogs, bivalirudin, desirudin, and lepirudin. Preferred direct factor IIa inhibitors include argatroban.

As used herein, an anti-proliferative agent refers to anti-proliferative agents, anti-mitotic agents, cytostatic agents and anti-migratory agents which suppress cell growth, proliferation, and/or metabolism. Examples of anti-proliferative agents include without limitation inhibitors of mammalian target of rapamycin (mTOR), rapamycin (also called sirolimus), deuterated rapamycin, rapamycin prodrug TAFA93, 40-O-alkyl-rapamycin derivatives. 40-O-hydroxyalkyl-rapamycin derivatives, everolimus {40-O-(2-hydroxyethyl)-rapamycin}. 40-O-(3-hydroxy) propyl-rapamycin. 40-O-[2-(2-hydroxy) ethoxy]ethyl-rapamycin. 40-O-alkoxyalkyl-rapamycin derivatives, biolimus {40-O-(2-ethoxyethyl)-rapamycin}. 40-O-acyl-rapamycin derivatives, temsirolimus {40-(3-hydroxy-2-hydroxymethyl-2-methylpropanoate)-rapamycin, or CCI-779 (temsirolimus). 40-O-phospho-containing rapamycin derivatives, ridaforolimus (40)-dimethylphosphinate-rapamycin, or AP23573 (ridaforolimus, formerly known as deforolimus). 40(R or S)-heterocyclyl- or heteroaryl-containing rapamycin derivatives, zotarolimus {40-epi-(N1-tetrazolyl)-rapamycin, or ABT-578 (zotarolimus). 40-epi-(N2-tetrazolyl)-rapamycin. 32 (R or S)-hydroxy-rapamycin, myolimus (32-deoxo-rapamycin), novolimus (16-O-desmethyl-rapamycin), taxanes, paclitaxel, docetaxel, cytochalasins, cytochalasins A through J, latrunculins, and salts, isomers, solvates, analogs (including deuterated analogs), derivatives, metabolites, and prodrugs thereof. The IUPAC numbering system for rapamycin is used herein. Preferred anti-proliferative agents include mTOR inhibitors and/or taxanes, or salts, isomers, solvates, analogs, derivatives, metabolites, or prodrugs thereof.

Table IA provides non-limiting examples of derivatives of each of rapamycin, everolimus, biolimus, temsirolimus, ridaforolimus, zotarolimus, myolimus and novolimus.

TABLE 1A
Derivatives of rapamycin-type compounds
Derivatives of Each of Rapamycin, Everolimus, Biolimus, Temsirolimus,
Ridaforolimus, Zotarolimus, Myolimus and Novolimus

N7-oxide
2-hydroxy
3-hydroxy
4-hydroxy
5-hydroxy
6-hydroxy
11-hydroxy
12-hydroxy
13-hydroxy
14-hydroxy
23-hydroxy
24-hydroxy
25-hydroxy
31-hydroxy
35-hydroxy
43-hydroxy (11-hydroxymethyl)
44-hydroxy (17-hydroxymethyl)
45-hydroxy (23-hydroxymethyl)
46-hydroxy (25-hydroxymethyl)
47-hydroxy (29-hydroxymethyl)
48-hydroxy (31-hydroxymethyl)
49-hydroxy (35-hydroxymethyl)
17,18-dihydroxy
19,20-dihydroxy
21,22-dihydroxy
29,30-dihydroxy
10-phosphate
28-phosphate
40-phosphate
16-O-desmethyl
27-O-desmethyl
39-O-desmethyl
16,27-bis(O-desmethyl)
16,39-bis(O-desmethyl)
27,39-bis(O-desmethyl)
16,27,39-tris(O-desmethyl)
16-desmethoxy
27-desmethoxy
39-O-desmethyl-14-hydroxy
17,18-epoxide
19,20-epoxide
21,22-epoxide
29,30-epoxide
17,18-29,30-bis-epoxide
17,18-19,20-21,22-tris-epoxide
19,20-21,22-29,30-tris-epoxide
16-O-desmethyl-17,18-19,20-bis-epoxide
16-O-desmethyl-17,18-29,30-bis-epoxide
16-O-desmethyl-17,18-19,20-21,22-tris-epoxide
16-O-desmethyl-19,20-21,22-29,30-tris-epoxide
27-O-desmethyl-17,18-19,20-21,22-tris-epoxide
39-O-desmethyl-17,18-19,20-21,22-tris-epoxide
16,27-bis(O-desmethyl)-17,18-19,20-21,22-tris-epoxide
16-O-desmethyl-24-hydroxy-17,18-19,20-bis-epoxide
16-O-desmethyl-24-hydroxy-17,18-29,30-bis-epoxide
12-hydroxy and opened hemiketal ring

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

In a preferred example, a device releasing one or more calcium chelating agent, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more cationic anti-coagulation enhancer, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more calcium chelating agent, one or more a calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more a calcium chelating agent, a factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In some examples, the injury to a tissue, surface, vessel/lumen wall, or other body part is the first substantial injury resulting from a surgery or intervention. In certain examples, the surgery or intervention is selected from the group consisting of vascular surgeries and interventions, cardiovascular surgeries and interventions, peripheral vascular surgeries and interventions, vascular grafting, vascular replacement, vascular angioplasty, thrombectomy, vascular stent placement, vascular laser therapy, coronary by-pass surgery, coronary angiography, coronary stent placement, carotid artery procedures, peripheral stent placement, organ transplants, artificial heart transplant, and plastic and cosmetic surgeries and interventions. In additional examples, the injury is the first substantial injury caused by the device delivering the one or more active substances, and optionally one or more other kinds of bioactive agents (e.g., anti-proliferative agents, anti-inflammatory agents, etc.). In some examples, a substantial injury to a tissue, surface, vessel/lumen wall or other body part results from contact of a device with the tissue, surface, vessel/lumen wall or other body part in a surgery or intervention (e.g., contact of the device causing damage to the endothelium lining a blood vessel, a surgical cutting instrument cutting a tissue, a deployed stent embedding into the wall of a blood vessel, etc.). In further examples, a substantial injury to a tissue, surface, vessel/lumen wall or other body part has a potential to elicit fibrin/thrombus formation, cell migration, cell proliferation or inflammation, or a combination thereof, at the site of injury or at an area adjacent thereto.

In some examples, the therapeutic composition is formulated to release the one or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device.

In some examples, each of the one or more active substances is released from a temporary or non-temporary device at a rate within a range of about 1 μg/hour/mm device length to about 30 μg/day/mm device length, for example about 1 μg/hour/mm device length to about 20 μg/day/mm device length. In some examples, the therapeutic composition is formulated to release the one or more active substances at a rate of 1 μg/hour/mm device to about 20 μg/day/mm device. In some examples, the therapeutic composition may be formulated to release the one or more active substances at a rate within a range of about 1 μg/hour/mm device length to about 14 μg/hour/mm device length. In some examples, the therapeutic composition may be formulated to release the one or more active substances at a rate within a range bounded by any two of the following values: about 1 μg/hour/mm device length, about 2 μg/hour/mm device length, about 3 μg/hour/mm device length, about 4 μg/hour/mm device length, about 5 μg/hour/mm device length, about 6 μg/hour/mm device length, about 7 μg/hour/mm device length, about 8 μg/hour/mm device length, about 9 μg/hour/mm device length, about 10) μg/hour/mm device length, about 11 μg/hour/mm device length, about 12 μg/hour/mm device length, about 13 μg/hour/mm device length, about 14 μg/hour/mm device length, about 15 μg/hour/mm device length, about 16 μg/hour/mm device length, about 17 μg/hour/mm device length, about 18 μg/hour/mm device length, about 19 μg/hour/mm device length, about 20 μg/hour/mm device length, about 21 μg/hour/mm device length, about 22 μg/hour/mm device length, about 23 μg/hour/mm device length, about 24 μg/hour/mm device length, about 25 μg/hour/mm device length, about 26 μg/hour/mm device length, about 27 μg/hour/mm device length, about 28 μg/hour/mm device length, about 29 μg/hour/mm device length, or about 30 μg/hour/mm device length.

In some examples, the therapeutic composition is formulated to begin releasing the one or more active substances within about 1 minute. 5, 10, 15, 20, 25, or 30 minutes after the external surface of the structure is positioned adjacent the injury site.

In some examples, substantially all of each of the one or more active substances is released from a temporary or non-temporary device within about 1 day to about 180 days or more, for example within about 1 day to about 90 days. In some examples, the therapeutic composition is formulated to release substantially all of the one or more active substances within about 7 days or about 28 days. In some examples, the therapeutic composition may be formulated to release substantially all of the one or more active substances within a range bounded by any two of the following values: 1 day. 3 days. 7 days. 14 days. 21 days. 28 days. 45 days. 90 days. 180 days, or more.

In some examples, the therapeutic composition is formulated to release substantially all of the one or more active substances within about 3 hours, about 6 hours, about 12 hours, about 1 day, or about 3 days. In some examples, the therapeutic composition is formulated to release at least 50%, at least 60%, or at least 70% of the one or more active substances within about 3 hours, about 6 hours, about 12 hours, about 1 day, about 3 days, about 7 days, or about 28 days.

In some examples, each of the one or more active substances is released from a temporary or non-temporary device at a rate sufficient to generate a tissue concentration of each of the agents within a range of about 5 ng/mg tissue to about 200 nm/mg tissue at the injury site within about 3 hours of tissue contact.

In some examples, the therapeutic composition is formulated to locally release the one or more active substances to the injury site at a rate sufficient to generate a tissue concentration of about 2 ng/mg tissue to about 800 ng/mg tissue, about 2 ng/mg tissue to about 200 ng/mg tissue, preferably at about 20 ng/mg tissue to about 200 ng/mg tissue, more preferably at about 40 ng/mg tissue to about 200 ng/mg tissue, of the one or more active substances at the injury site within about 3 hours after the external surface of the structure is positioned adjacent the injury site. In some examples, the therapeutic composition is formulated to release the one or more active substances at a rate sufficient to generate a tissue concentration at the injury site within a range of about 10 ng/mg tissue to about 100 ng/mg tissue. The therapeutic composition may be formulated to locally release the one or more active substances to the injury site at a rate sufficient to generate a tissue concentration of the one or more active substances at the injury site within about 3 hours after placement adjacent the injury site within a range bounded by any two of the following values: 2 ng/mg tissue, 5 ng/mg tissue, 10 ng/mg tissue, 20 ng/mg tissue, 30 ng/mg tissue. 40 ng/mg tissue. 50 ng/mg tissue, 60 ng/mg tissue. 70 ng/mg tissue, 80 ng/mg tissue, 90 ng/mg tissue. 100 ng/mg tissue, 110 ng/mg tissue, 120 ng/mg tissue, 130 ng/mg tissue, 140 ng/mg tissue, 150 ng/mg tissue, 160 ng/mg tissue, 170 ng/mg tissue, 180 ng/mg tissue, 190 ng/mg tissue, or 200 ng/mg tissue.

In another example, the device releases the one or more active substances from 1 microgram per mm of device length to 25 micrograms per mm of device length, and preferably releases said agent from 5 micrograms per mm of device length to 20 micrograms per mm of device length.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm2 device to about 15 μg/mm2 device, or of about 1 μg/mm2 device to about 12 μg/mm2 device, or of about 2 μg/mm2 device to about 12 μg/mm2 device, or of about 5 μg/mm2 device to about 12 μg/mm2 device, or of about 7 μg/mm2 device to about 12 μg/mm2 device, within about 3 hours or about 12 hours or about 1 day or about 3 days or about 7 days. In some examples, the therapeutic composition is formulated to release the one or more active substances at a dose within a range of about 1 μg/mm2 device to about 12 μg/mm2 device within about 3 hours. In some examples, the therapeutic composition is formulated to release the one or more active substances at a dose within a range of about 1 μg/mm2 device to about 12 μg/mm2 device within about 12 hours. In some examples, the therapeutic composition is formulated to release the one or more active substances at a dose within a range of about 1 μg/mm2 device to about 12 μg/mm2 device within about 7 days. In some examples, the therapeutic composition is formulated to release the one or more active substances at a dose within a range of about 1 μg/mm2 device to about 12 μg/mm2 device within about 28 days, about 90 days, or about 180) days.

In some examples, each of the one or more agents is released from a temporary or non-temporary device at a rate sufficient to generate a tissue concentration of each of the agents within a range of about 1 ng/mg tissue at about 100 ng/mg tissue within about 28 days of tissue contact.

In some examples, the therapeutic composition is formulated to release the one or more active substances at a rate sufficient to generate a tissue concentration of about 0.5 ng/mg to about 10 ng/mg within the tissue adjacent to the device structure within about 28 days, about 90 days, or about 180 days.

In some examples, the therapeutic composition is formulated to release the one or more active substances at a rate sufficient to generate a tissue concentration at the injury site within a range of about 0.5 ng/mg to about 30 ng/mg within about 28 days. In some examples, the therapeutic composition is formulated to release the one or more active substances at a rate sufficient to generate a tissue concentration at the injury site within a range of about 1 ng/mg to about 20 ng/mg within about 28 days. In some examples, the therapeutic composition is formulated to release the one or more active substances at a rate sufficient to generate a tissue concentration at the injury site within a range of about 1.5 ng/mg to about 25 ng/mg within about 28 days.

In some examples, the therapeutic composition is formulated to release the one or more active substances at a rate sufficient to generate a tissue concentration at the injury site within a range of about 0.1 ng/mg to about 10 ng/mg within about 90 days or about 180 days.

In some examples, each of the one or more agents is released from a temporary or non-temporary device at the same rate. In other examples, one or more of the one or more agents that inhibit fibrin/thrombus formation or promote fibrin/thrombus dissolution and/or other bioactive agents is released from a temporary or non-temporary device at a different rate.

In some examples, the therapeutic composition is formulated to release the calcium chelating agent, direct factor Xa inhibitor and/or the direct factor IIa inhibitor faster than the anti-proliferative agent.

In some examples, the therapeutic composition is formulated to release a larger dose of a calcium chelating agent, the direct factor Xa inhibitor than the anti-proliferative agent. In some examples, the dose of a calcium chelating agent, the direct factor Xa inhibitor is about 1.25 to about 5 times larger, about 1.5 to about 3 times larger, or about 1.5 to about 2.5 times larger than a dose of the anti-proliferative agent.

In some examples, the therapeutic composition is formulated to release the one or more active substances at a rate sufficient to generate a tissue concentration at a location proximal or distal a proximal end of the structure or a distal end of the structure, respectively (e.g., an adjacent tissue segment), within a range of about 0.5 ng/mg to about 500 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about 1 ng/mg to about 35 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about a range of about 1.5 ng/mg to about 30 ng/mg within about 3 hours. In some examples, the therapeutic composition is formulated to release the one or more active substances at a rate sufficient to generate a tissue concentration at the location proximal or distal to the proximal end of the structure or the distal end of the structure (e.g., within +5 mm proximal or distal to an end of the structure), respectively, within a range of about 0.1 ng/mg to about 50 ng/mg, about 0.25 ng/mg to about 20 ng/mg, about 1 ng/mg to about 50) ng/mg, or about 3 ng/mg to about 50 ng/mg within about 3 hours.

In some examples, the therapeutic composition is formulated to release the one or more active substances at a rate sufficient to generate a tissue concentration at a location proximal or distal a proximal end of the structure or a distal end of the structure, respectively, within a range of about 0.2 ng/mg to about 25 ng/mg, about 2 ng/mg to about 25 ng/mg, or about 4 ng/mg to about 25 ng/mg within about 24 hours. In some examples, the therapeutic composition is formulated to release the one or more active substances at a rate sufficient to generate a tissue concentration at the location proximal or distal to the proximal end of the structure or the distal end of the structure (e.g., within +5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 24 hours. In some examples, the therapeutic composition is formulated to release the one or more active substances at a rate sufficient to generate a tissue concentration at the location proximal or distal the proximal end of the structure or the distal end of the structure, respectively, within a range of about 0.3 ng/mg to about 10 ng/mg within about 24 hours.

In some examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cmin) of the direct factor Xa inhibitor generated by systemic delivery. In some examples, the blood concentration is smaller than a median minimum serum concentration (Cmin) of the direct factor Xa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC(0-∞)) in ng·h/ml which is smaller than a median (AUC(0-24) or AUC(0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC(0-24) or AUC(0-∞)) in ng·h/ml which is smaller than a median (AUC(0-24) or AUC(0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC(0-24) or AUC(0-∞)) is measured using one of plasma blood, serum blood, or whole blood. In other examples, the median (AUC(0-24) or AUC(0-∞)) is 724 ng·h/ml, or1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cmax) of the anti-proliferative agent generated by systemic delivery of the anti-proliferative agent to achieve the same tissue concentration at the injury site. In some examples, the systemic delivery comprises a single oral dose, a daily oral dose, or a smallest oral dose of the anti-proliferative agent. In some examples, the blood concentration is larger than a median minimum serum concentration (Cmin) of the anti-proliferative agent generated by systemic delivery. In some examples, the blood concentration is smaller than a median minimum serum concentration (Cmin) of the anti-proliferative agent generated by systemic delivery. In some examples, the therapeutic composition is formulated to release a dose of the anti-proliferative agent sufficient to generate a plasma drug level area under the curve (AUC(0-∞)) in ng·h/ml which is smaller than a median AUC(0-∞) in ng·h/ml of the anti-proliferative agent generated by systemic delivery of the anti-proliferative agent to achieve the same tissue concentration at the injury site.

In some examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cmin) of the direct factor IIa inhibitor generated by systemic delivery. In some examples, the blood concentration is smaller than a median minimum serum concentration (Cmin) of the direct factor IIa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0)-24) or AUC(0-∞)) in ng·h/ml which is smaller than a median (AUC(0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC(0-24) or AUC(0-∞)) in ng·h/ml which is smaller than a median (AUC(0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC(0-24) or AUC (0-∞)) is measured using one of plasma blood, serum blood, or whole blood. In other examples, the median (AUC(0-24) or AUC(0-∞)) is 724 ng·h/ml, or 1437 ng·h/ml, or 2000 ng·h/ml, or 4000 ng·h/ml.

In some examples, local delivery of one or more of the active substances may reduce the time a patient needs to spend on oral medications and/or obviate the need for such medications entirely.

In some examples, the dose of each of the one or more active substances for optional systemic administration on a one-time basis or over a certain time period described herein (e.g., 6 hr. 12 hr. 1 day, 2 days. 3 days. 1 week. 2 weeks, 3 weeks. 1 month, etc.) independently is at least about 1, 5, 10, 20, 50, 100 or 500 mg, or at least about 1, 5 or 10 g. In further examples, the amount of each of the one or more active substances loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the fibrin/thrombus formation-inhibiting or fibrin/thrombus dissolution-promoting agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg.

In further examples, the concentration of each of the one or more active substances released from a temporary or non-temporary device, and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50, 100, 500 or 1000 μM. In certain examples, the concentration of each of the fibrin/thrombus formation-inhibiting or fibrin/thrombus dissolution-promoting agent(s) released from a temporary or non-temporary device, and optionally administered systemically in addition to locally, in blood or tissue at the site of injury or at an area adjacent thereto, and/or in blood or tissue adjacent to the device, independently is about 0.01 or 0). 1 nM to about 1000 μM, or about 0.1 or 1 nM to about 500 μM, or about 1 or 10 nM to about 100 μM, or about 50 nM to about 50 μM, or about 10 or 100 nM to about 10 μM, or about 100 nM to about 1 μM, or about 1 μM to about 10 μM.

In still further examples, the concentration of each of the one or more active substances released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the fibrin/thrombus formation-inhibiting or fibrin/thrombus dissolution-promoting agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50) μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In additional examples, the concentration of each of the one or more active substances released from a temporary or non-temporary device (that may or may not cause an injury to a tissue, surface, vessel/lumen wall or other body part), and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50 or 100 μM, within about 1 day, 12 hr, 6 hr, 3 hr, 2 hr, 1 hr. 30 min., 15 min., 5 min, or 1 min, before, during and/or within about 1 day, 12 hr, 6 hr, 3 hr, 2 hr, 1 hr. 30 min., 15 min., 5 min, or 1 min, after delivery or deployment of the device and/or the injury. In further examples, the concentration of each of the one or more active substances released from a temporary or non-temporary device (that may or may not cause an injury to a tissue, surface, vessel/lumen wall or other body part), and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50 or 100 μg/gm tissue, within about 1 day, 12 hr, 6 hr, 3 hr, 2 hr, 1 hr. 30 min., 15 min., 5 min, or 1 min, before, during and/or within about 1 day, 12 hr, 6 hr, 3 hr, 2 hr. 1 hr, 30 min., 15 min., 5 min, or 1 min, after delivery or deployment of the device and/or the injury.

In some examples, the dose of each of the one or more optional other kinds of bioactive agents (e.g., anti-proliferative agents, anti-inflammatory agents, etc.) for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In additional examples, the amount of each of the one or more optional other kinds of bioactive agents (e.g., anti-proliferative agents, anti-inflammatory agents, etc.) loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the optional other kind(s) of bioactive agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg, or about 50 μg to about 200 μg.

In further examples, the concentration of each of the one or more optional other kinds of bioactive agents (e.g., anti-proliferative agents, anti-inflammatory agents, etc.) released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the optional other kind(s) of bioactive agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50) ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In some examples, the device contains the bioactive agent(s) in the body and/or on at least one surface of the device. In certain examples, the bioactive agent(s) are contained in one or more layers in the body and/or at the surface of the device.

In further examples, the bioactive agent(s) are contained in one or more coatings disposed over the body of the device. The coating(s) can be disposed over any desired portion(s) and any desired surface(s) of the body of the device. As a non-limiting example, for a tubular vascular device such as a stent, the coating(s) can be disposed over the luminal (lumen-facing) surface, the abluminal (tissue-facing) surface or the side surface(s) of the stent, or a combination thereof (e.g., all surfaces of the stent).

In additional examples, the device comprises the bioactive agent(s) in the body of the device and in one or more coatings disposed over the body of the device.

A temporary or non-temporary device can comprise openings in and/or on the body (including at the surface) of the device, and/or in one or more coatings disposed over the body structure of the device. Examples of openings include without limitation pores (including partial pores and through pores), holes (including partial holes and through holes), voids, recesses, pits, cavities, trenches, reservoirs and channels. In some examples, a temporary or non-temporary device contains one or more anti-coagulant, and optionally one or more other kinds of bioactive agents (e.g., anti-proliferative agents, anti-inflammatory agents, etc.) in openings in and/or on the body (including at the surface) of the device, and/or in one or more coatings disposed over the body of the device.

The device may comprise one or more coatings disposed over an exterior surface of a structure of the device, as described herein. In some embodiments, the coating(s) may comprise a homopolymer, a copolymer, a mixture of homopolymers, a mixture of copolymers, or a mixture of a homopolymer and a copolymer. In some examples, the coating(s) comprise a soft or hydrophilic, or a softer or more hydrophilic, polymeric material. In further examples, the coating(s) comprise a polymeric material and an additive (e.g., a monomer of the polymeric material) that softens the polymeric material.

In some examples, the device has a first coating that comprises a biodegradable or non-degradable polymeric material, or one or more bioactive agents, or both a biodegradable or non-degradable polymeric material and one or more bioactive agents. In further examples, the device has a second coating that comprises a biodegradable or non-degradable polymeric material, or one or more bioactive agents, or both a biodegradable or non-degradable polymeric material and one or more bioactive agents, wherein the second coating optionally is disposed over the first coating. In additional examples, the device has a third coating that comprises a biodegradable or non-degradable polymeric material, wherein the third coating is disposed over the first coating and/or the second coating. In some examples, the third coating serves as a top layer or coat or diffusion barrier that controls release of one or more bioactive agents from inner coating(s) and/or the body of the device.

In some examples, a bioactive agent that is intended to have an earlier or shorter time of action can be contained in an outer coating, on a surface uncovered by a coating, and/or in the body of the device closer to the surface, and a bioactive agent that is intended to have a later or longer time of action can be contained in an inner coating, in a coating covered by a barrier coating, on a surface covered by a coating, and/or in the body of the device farther from the surface. In further examples, a bioactive agent that is intended to have an earlier or shorter time of action is contained on a surface of the device, or contained in a coating on the device or in a layer of the body of the device which comprises a faster-degrading polymeric material, and a bioactive agent that is intended to have a later or longer time of action is contained within the device, or contained in a coating on the device or in a layer of the body of the device which comprises a slower-degrading or non-degrading polymeric material. In additional examples, a bioactive agent that is intended to have an earlier or shorter time of action is more soluble, and a bioactive agent that is intended to have a later or longer time of action is less soluble.

In certain examples, the concentration of a bioactive agent [e.g., anti-coagulant, anti-proliferative, etc.] in a coating comprising a polymeric material is at least about 10%, 20%, 30%, 40%, 50% or 60% by weight relative to the weight of the bioactive agent and the polymeric material.

In further examples, the thickness (e.g., average thickness) of each of the coating(s) independently is no more than about 20, 15, 10, 5, 3 or 1 micron.

In some examples, the coating(s) may comprise carrier material. Non-limiting examples of carrier materials include biodegradable polymeric materials, non-degradable polymeric materials, and other matrix materials.

In some examples, the carrier material may be porous. In certain examples, the porosity of each of the coating(s) of the carrier material may be within a range of about 10 nm to about 10 μm.

In some examples, the carrier material may be biodegradable. In certain examples, the carrier material may have a depredation rate within a range of about 1 month to about 36 months.

In some examples, the weight compositional ratio of the carrier material to the therapeutic composition of one or more bioactive agents may be within a range of about 1:5 to 3:2.

Non-limiting examples of polymeric materials that can compose the carrier material include polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-8-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide. D-lactide and D,L-lactide. The polymeric material may comprise a material selected from a group of non-degradable polymeric materials consisting of polyacrylates, polymethacrylates, poly(n-butyl methacrylate), poly(hydroxyethylmethacrylate), polyamides, nylons, nylon 12. Dacron. Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacry loy loxyethylphosphorylcholine-co-butyl methacrylate), and copolymers and combinations thereof.

Non-limiting examples of biodegradable polymeric materials that can compose the body of the device, a layer of the body, or a coating include polyesters, poly(α-hydroxyacids), polylactide, polyglycolide, poly(ε-caprolactone), polydioxanone, poly(hydroxyalkanoates), poly(hydroxypropionates), poly(3-hydroxypropionate), poly(hydroxy butyrates), poly(3-hydroxy butyrate), poly(4-hydroxy butyrate), poly(hydroxypentanoates), poly(3-hydroxypentanoate), poly(hydroxyvalerates), poly(3-hydroxyvalerate), poly(4-hydroxyvalerate), poly(hydroxyoctanoates), poly(3-hydroxyoctanoate), polysalicylate/polysalicylic acid, polycarbonates, poly(trimethylene carbonate), poly(ethylene carbonate), poly(propylene carbonate), tyrosine-derived polycarbonates. L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1.6HD), poly(DETOSU-t-CDM), polyurethanes, polyphosphazenes, polyamides, nylons, nylon 12, polyoxyethylated castor oil, poly(ethylene glycol), polyethylene oxide (PEO), polyvinylpyrrolidone, poly(L-lactide-co-D-lactide), ethylene-vinyl acetate, poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-8-caprolactone) (including 70): 30 to 99:1 PLA-co-PCL, such as 90:10 PLA-co-PCL), poly(glycolide-co-8-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxy butyrate), poly(lactide-co-3-hydroxy butyrate), poly(lactide-co-4-hydroxy butyrate), poly(glycolide-co-hydroxy butyrate), poly(glycolide-co-3-hydroxy butyrate), poly(glycolide-co-4-hydroxy butyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxy butyrate-co-4-hydroxy butyrate), poly(hydroxy butyrate-co-hydroxyvalerate), poly(3-hydroxy butyrate-co-3-hydroxyvalerate), poly(3-hydroxy butyrate-co-4-hydroxyvalerate), poly(4-hydroxy butyrate-co-3-hydroxyvalerate), poly(4-hydroxy butyrate-co-4-hydroxyvalerate), poly(ε-caprolactone-co-fumarate), poly(ε-caprolactone-co-propylene fumarate), poly(ester-co-ether), poly(lactide-co-ethylene glycol), poly(glycolide-co-ethylene glycol), poly(ε-caprolactone-co-ethylene glycol), poly(ester-co-amide), poly(DETOSU-1.6HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-8-caprolactone), poly(lactide-co-glycolide-co-trimethylene carbonate), poly(lactide-co-8-caprolactone-co-trimethylene carbonate), poly(glycolide-co-8-caprolactone-co-trimethylene carbonate), poly(3-hydroxy butyrate-co-3-hydroxyvalerate-co-4-hydroxy butyrate), poly(3-hydroxy butyrate-co-4-hydroxyvalerate-co-4-hydroxy butyrate), collagen, casein, polysaccharides, cellulose, cellulose esters, cellulose acetate, cellulose butyrate, cellulose acetate butyrate, cellulose propionate, chitin, chitosan, dextran, starch, modified starch, and copolymers and combinations thereof, wherein lactide includes L-lactide. D-lactide and D,L-lactide.

Examples of non-degradable polymeric materials that can compose the body of the device, a layer of the body, or a coating include without limitation polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene). Blend of polyvinylpyrrolidone, poly(hexylmethacrylate)-co-polyvinylpyrrolidone-co-poly vinyl acetate, and poly(n-butyl methacrylate)-co-poly(vinyl acetate). Poly(styrene-butylene styrene), poly(tyrosine-derived polycarbonate), polyamides, nylons, nylon 12, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate).), polyvinylpyridine block with poly methyl methacrylate (PMMA), poly N-(2-Hydroxypropyl) methacrylamide. Polyethylenimine (PEI), dextran, dextrin, chitosans, poly(L-lysine), and poly(aspartamides), polyamides. Polyethylene glycol (PEG). Silicones, poly(anhydride), poly ortho esters, polystyrene-b-polyvinylpyridine, poly(styrene)-poly (butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-poly(ethylene oxide) and copolymers and/or combinations thereof.

Non-limiting examples of corrodible metals and metal alloys that can compose the body of the device, a layer of the body, or a coating include cast ductile irons (e.g., 80-55-06 grade cast ductile iron), corrodible steels (e.g., AISI 1010) steel, AISI 1015 steel, AISI 1430 steel, AISI 5140 steel and AISI 8620 steel), melt-fusible metal alloys, bismuth-tin alloys (e.g., 40% bismuth-60% tin and 58% bismuth-42% tin), bismuth-tin-indium alloys, magnesium, magnesium alloys, tungsten alloys, zinc alloys, shape-memory metal alloys, and superelastic metal alloys. Examples of non-corrodible metals and metal alloys that can compose the body of the device, a layer of the body, or a coating include without limitation stainless steels (e.g., 316L stainless steel), cobalt-chromium alloys (e.g., L-605 and MP35N cobalt-chromium alloys), gold, molybdenum-rhenium alloys, nickel-titanium alloys, palladium, platinum, platinum-iridium alloys, tantalum, and alloys thereof.

In some examples, the device is coated. The coating layer may comprise a therapeutic agent and an additive. In some examples, the coating layer overlying an exterior surface of the exterior surface of the medical device consists essentially of the therapeutic agent and the additive.

In some examples, the additive is selected from PEG (polyethylene glycol), polyalkylene oxide, e.g., polyethylene oxide, polypropylene oxide, or a copolymer thereof (e.g., a polyethylene oxide-polypropylene oxide-polyethylene oxide copolymers), polyphenylene oxide, copolymers of PEG and polyalkylene oxide, poly(methoxyethyl methacrylate benzoate), poly(a methacryloyloxy one phosphatidylcholine), perfluorinated polyether, dextran or poly vinylpyrrolidone, poly(ethylene-vinyl acetate), polypeptides, water soluble surfactants, water soluble vitamins, and proteins, PEG fatty esters and alcohols, glycerol fatty esters, sorbitan fatty esters, PEGylation (PEG-drug conjugation), PEG glyceryl fatty esters, PEG sorbitan fatty esters, sugar fatty esters, PEG sugar esters, vitamins and derivatives, amino acids, multi amino acids and derivatives, peptides, polypeptides, oligomers, copolymers, block polymers, proteins, albumin. quaternary ammonium salts such as but not limted to benzalkonium chloride, benzethonium chloride, docecyl trimethyl ammonium bromide, sodium docecylsulfates, dialkyl methylbenzyl ammonium chloride, dialkylesters of sodium sulfonsuccinic acid, organic acids, salts and anhydrides and combinations thereof.

In another example, a factor Xa inhibitor, preferably Rivaroxaban, more preferably Apixaban released locally in combination with Argatroban from an implant to inhibit fibrin or clot formation.

In another example, a factor Xa inhibitor, preferably Rivaroxaban, more preferably Apixaban released locally in combination with Argatroban from an implant to inhibit fibrin, clot formation, and/or smooth muscle cell proliferation.

In another example, a factor Xa inhibitor, preferably Rivaroxaban, more preferably Apixaban released locally by an implant to inhibit clot formation.

In one example, a device delivery one or more drugs locally, wherein locally comprises delivering said one or more drugs to one or more of site specific location, to a vessel wall, adjacent to a vessel wall, in a body lumen, to a body organ, within a body organ, to the device surface in a body lumen, to a tissue, or to an injured tissue.

In another example, a factor Xa inhibitor, preferably Rivaroxaban, more preferably Apixaban is released locally in combination with an m-TOR inhibitor to inhibit fibrin formation or clot formation, or to inhibit fibrin formation or clot formation through 7 days, or to inhibit fibrin formation or clot formation through 28 days.

In another example, a third antiproliferative drug is configured to be released from the device in combination with Argatroban and Rivaroxaban or Apixaban, at similar dose and release rate or different dose and release rate. In a specific example, the anti-proliferative drug is sirolimus or its analogs (including deuterated analog), metabolites, or salts.

In one example, a device delivery one or more drugs locally, wherein locally comprises delivery of said one or more drugs to one or more of site specific location, adjacent to a vessel wall, to a vessel wall, in a body lumen, to the device surface in a body lumen, to a tissue, to an injured tissue, wherein the local concentration of the one or more dugs maybe higher than in the systemic concentration of the one or more drugs.

In a preferred example, a device releasing factor Xa inhibitor in a body lumen wherein said device inhibits fibrin formation thereby inhibiting clot formation.

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

In a preferred example, a device releasing one or more calcium chelating agent, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more cationic anti-coagulation enhancer, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more calcium chelating agent, one or more a calcium chelating agent of EDTA ammonium slat complex, factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In a preferred example, a device releasing one or more a calcium chelating agent, a factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In some examples, the injury to a tissue, surface, vessel/lumen wall, or other body part is the first substantial injury resulting from a surgery or intervention. In certain examples, the surgery or intervention is selected from the group consisting of vascular surgeries and interventions, cardiovascular surgeries and interventions, peripheral vascular surgeries and interventions, vascular grafting, vascular replacement, vascular angioplasty, thrombectomy, vascular stent placement, vascular laser therapy, coronary by-pass surgery, coronary angiography, coronary stent placement, carotid artery procedures, peripheral stent placement, organ transplants, artificial heart transplant, and plastic and cosmetic surgeries and interventions. In additional examples, the injury is the first substantial injury caused by the device delivering the one or more active substances, and optionally one or more other kinds of bioactive agents (e.g., anti-proliferative agents, anti-inflammatory agents, etc.). In some examples, a substantial injury to a tissue, surface, vessel/lumen wall or other body part results from contact of a device with the tissue, surface, vessel/lumen wall or other body part in a surgery or intervention (e.g., contact of the device causing damage to the endothelium lining a blood vessel, a surgical cutting instrument cutting a tissue, a deployed stent embedding into the wall of a blood vessel, etc.). In further examples, a substantial injury to a tissue, surface, vessel/lumen wall or other body part has a potential to elicit fibrin/thrombus formation, cell migration, cell proliferation or inflammation, or a combination thereof, at the site of injury or at an area adjacent thereto.

In some examples, the therapeutic composition is formulated to release the one or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device.

In some examples, each of the one or more active substances is released from a temporary or non-temporary device at a rate within a range of about 1 μg/hour/mm device length to about 30 μg/day/mm device length, for example about 1 μg/hour/mm device length to about 20 μg/day/mm device length. In some examples, the therapeutic composition is formulated to release the one or more active substances at a rate of 1 μg/hour/mm device to about 20 μg/day/mm device. In some examples, the therapeutic composition may be formulated to release the one or more active substances at a rate within a range of about 1 μg/hour/mm device length to about 14 μg/hour/mm device length. In some examples, the therapeutic composition may be formulated to release the one or more active substances at a rate within a range bounded by any two of the following values: about 1 μg/hour/mm device length, about 2 μg/hour/mm device length, about 3 μg/hour/mm device length, about 4 μg/hour/mm device length, about 5 μg/hour/mm device length, about 6 μg/hour/mm device length, about 7 μg/hour/mm device length, about 8 μg/hour/mm device length, about 9 μg/hour/mm device length, about 10 μg/hour/mm device length, about 11 μg/hour/mm device length, about 12 μg/hour/mm device length, about 13 μg/hour/mm device length, about 14 μg/hour/mm device length, about 15 μg/hour/mm device length, about 16 μg/hour/mm device length, about 17 μg/hour/mm device length, about 18 μg/hour/mm device length, about 19 μg/hour/mm device length, about 20 μg/hour/mm device length, about 21 μg/hour/mm device length, about 22 μg/hour/mm device length, about 23 μg/hour/mm device length, about 24 μg/hour/mm device length, about 25 μg/hour/mm device length, about 26 μg/hour/mm device length, about 27 μg/hour/mm device length, about 28 μg/hour/mm device length, about 29 μg/hour/mm device length, or about 30) μg/hour/mm device length.

In some examples, the therapeutic composition is formulated to begin releasing the one or more active substances within about 1 minute, 5, 10, 15, 20, 25, or 30 minutes after the external surface of the structure is positioned adjacent the injury site.

In some examples, substantially all of each of the one or more active substances is released from a temporary or non-temporary device within about 1 day to about 180 days or more, for example within about 1 day to about 90 days. In some examples, the therapeutic composition is formulated to release substantially all of the one or more active substances within about 7 days or about 28 days. In some examples, the therapeutic composition may be formulated to release substantially all of the one or more active substances within a range bounded by any two of the following values: 1 day, 3 days, 7 days, 14 days, 21 days, 28 days, 45 days, 90 days, 180 days, or more.

In some examples, the therapeutic composition is formulated to release substantially all of the one or more active substances within about 3 hours, about 6 hours, about 12 hours, about 1 day, or about 3 days. In some examples, the therapeutic composition is formulated to release at least 50%, at least 60%, or at least 70% of the one or more active substances within about 3 hours, about 6 hours, about 12 hours, about 1 day, about 3 days, about 7 days, or about 28 days.

In some examples, each of the one or more active substances is released from a temporary or non-temporary device at a rate sufficient to generate a tissue concentration of each of the agents within a range of about 5 ng/mg tissue to about 200 nm/mg tissue at the injury site within about 3 hours of tissue contact.

In some examples, the therapeutic composition is formulated to locally release the one or more active substances to the injury site at a rate sufficient to generate a tissue concentration of about 2 ng/mg tissue to about 800 ng/mg tissue, about 2 ng/mg tissue to about 200 ng/mg tissue, preferably at about 20 ng/mg tissue to about 200 ng/mg tissue, more preferably at about 40) ng/mg tissue to about 200 ng/mg tissue, of the one or more active substances at the injury site within about 3 hours after the external surface of the structure is positioned adjacent the injury site. In some examples, the therapeutic composition is formulated to release the one or more active substances at a rate sufficient to generate a tissue concentration at the injury site within a range of about 10 ng/mg tissue to about 100 ng/mg tissue. The therapeutic composition may be formulated to locally release the one or more active substances to the injury site at a rate sufficient to generate a tissue concentration of the one or more active substances at the injury site within about 3 hours after placement adjacent the injury site within a range bounded by any two of the following values: 2 ng/mg tissue, 5 ng/mg tissue, 10) ng/mg tissue, 20 ng/mg tissue, 30) ng/mg tissue, 40 ng/mg tissue, 50) ng/mg tissue, 60 ng/mg tissue. 70 ng/mg tissue, 80 ng/mg tissue, 90 ng/mg tissue. 100 ng/mg tissue, 110 ng/mg tissue, 120 ng/mg tissue. 130) ng/mg tissue, 140 ng/mg tissue. 150) ng/mg tissue, 160 ng/mg tissue, 170) ng/mg tissue, 180 ng/mg tissue, 190) ng/mg tissue, or 200 ng/mg tissue.

In another example, the device releases the one or more active substances from 1 microgram per mm of device length to 25 micrograms per mm of device length, and preferably releases said agent from 5 micrograms per mm of device length to 20 micrograms per mm of device length.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm2 device to about 15 μg/mm2 device, or of about 1 μg/mm2 device to about 12 μg/mm2 device, or of about 2 μg/mm2 device to about 12 μg/mm2 device, or of about 5 μg/mm2 device to about 12 μg/mm2 device, or of about 7 μg/mm2 device to about 12 μg/mm2 device, within about 3 hours or about 12 hours or about 1 day or about 3 days or about 7 days. In some examples, the therapeutic composition is formulated to release the one or more active substances at a dose within a range of about 1 μg/mm2 device to about 12 μg/mm2 device within about 3 hours. In some examples, the therapeutic composition is formulated to release the one or more active substances at a dose within a range of about 1 μg/mm2 device to about 12 μg/mm2 device within about 12 hours. In some examples, the therapeutic composition is formulated to release the one or more active substances at a dose within a range of about 1 μg/mm2 device to about 12 μg/mm2 device within about 7 days. In some examples, the therapeutic composition is formulated to release the one or more active substances at a dose within a range of about 1 μg/mm2 device to about 12 μg/mm2 device within about 28 days, about 90 days, or about 180 days.

In some examples, each of the one or more agents is released from a temporary or non-temporary device at a rate sufficient to generate a tissue concentration of each of the agents within a range of about 1 ng/mg tissue at about 100 ng/mg tissue within about 28 days of tissue contact.

In some examples, the therapeutic composition is formulated to release the one or more active substances at a rate sufficient to generate a tissue concentration of about 0.5 ng/mg to about 10 ng/mg within the tissue adjacent to the device structure within about 28 days, about 90 days, or about 180 days.

In some examples, the therapeutic composition is formulated to release the one or more active substances at a rate sufficient to generate a tissue concentration at the injury site within a range of about 0.5 ng/mg to about 30 ng/mg within about 28 days. In some examples, the therapeutic composition is formulated to release the one or more active substances at a rate sufficient to generate a tissue concentration at the injury site within a range of about 1 ng/mg to about 20 ng/mg within about 28 days. In some examples, the therapeutic composition is formulated to release the one or more active substances at a rate sufficient to generate a tissue concentration at the injury site within a range of about 1.5 ng/mg to about 25 ng/mg within about 28 days.

In some examples, the therapeutic composition is formulated to release the one or more active substances at a rate sufficient to generate a tissue concentration at the injury site within a range of about 0.1 ng/mg to about 10 ng/mg within about 90 days or about 180 days.

In some examples, each of the one or more agents is released from a temporary or non-temporary device at the same rate. In other examples, one or more of the one or more agents that inhibit fibrin/thrombus formation or promote fibrin/thrombus dissolution and/or other bioactive agents is released from a temporary or non-temporary device at a different rate.

In some examples, the therapeutic composition is formulated to release the calcium chelating agent, direct factor Xa inhibitor and/or the direct factor IIa inhibitor faster than the anti-proliferative agent.

In some examples, the therapeutic composition is formulated to release a larger dose of a calcium chelating agent, the direct factor Xa inhibitor than the anti-proliferative agent. In some examples, the dose of a calcium chelating agent, the direct factor Xa inhibitor is about 1.25 to about 5 times larger, about 1.5 to about 3 times larger, or about 1.5 to about 2.5 times larger than a dose of the anti-proliferative agent.

In some examples, the therapeutic composition is formulated to release the one or more active substances at a rate sufficient to generate a tissue concentration at a location proximal or distal a proximal end of the structure or a distal end of the structure, respectively (e.g., an adjacent tissue segment), within a range of about 0.5 ng/mg to about 500 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about 1 ng/mg to about 35 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about a range of about 1.5 ng/mg to about 30 ng/mg within about 3 hours. In some examples, the therapeutic composition is formulated to release the one or more active substances at a rate sufficient to generate a tissue concentration at the location proximal or distal to the proximal end of the structure or the distal end of the structure (e.g., within +5 mm proximal or distal to an end of the structure), respectively, within a range of about 0.1 ng/mg to about 50 ng/mg, about 0.25 ng/mg to about 20 ng/mg, about 1 ng/mg to about 50) ng/mg, or about 3 ng/mg to about 50 ng/mg within about 3 hours.

In some examples, the therapeutic composition is formulated to release the one or more active substances at a rate sufficient to generate a tissue concentration at a location proximal or distal a proximal end of the structure or a distal end of the structure, respectively, within a range of about 0.2 ng/mg to about 25 ng/mg, about 2 ng/mg to about 25 ng/mg, or about 4 ng/mg to about 25 ng/mg within about 24 hours. In some examples, the therapeutic composition is formulated to release the one or more active substances at a rate sufficient to generate a tissue concentration at the location proximal or distal to the proximal end of the structure or the distal end of the structure (e.g., within +5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 24 hours. In some examples, the therapeutic composition is formulated to release the one or more active substances at a rate sufficient to generate a tissue concentration at the location proximal or distal the proximal end of the structure or the distal end of the structure, respectively, within a range of about 0.3 ng/mg to about 10 ng/mg within about 24 hours.

In some examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cmax) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cmin) of the direct factor Xa inhibitor generated by systemic delivery. In some examples, the blood concentration is smaller than a median minimum serum concentration (Cmin) of the direct factor Xa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0)-24) or AUC(0-∞)) in ng·h/ml which is smaller than a median (AUC(0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC(0-24) or AUC(0-∞)) in ng·h/ml which is smaller than a median (AUC(0-24) or AUC(0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC(0-24) or AUC (0-∞)) is measured using one of plasma blood, serum blood, or whole blood. In other examples, the median (AUC(0-24) or AUC(0-∞)) is 724 ng·h/ml, or1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cmax) of the anti-proliferative agent generated by systemic delivery of the anti-proliferative agent to achieve the same tissue concentration at the injury site. In some examples, the systemic delivery comprises a single oral dose, a daily oral dose, or a smallest oral dose of the anti-proliferative agent. In some examples, the blood concentration is larger than a median minimum serum concentration (Cmin) of the anti-proliferative agent generated by systemic delivery. In some examples, the blood concentration is smaller than a median minimum serum concentration (Cmin) of the anti-proliferative agent generated by systemic delivery. In some examples, the therapeutic composition is formulated to release a dose of the anti-proliferative agent sufficient to generate a plasma drug level area under the curve (AUC(0-∞)) in ng·h/ml which is smaller than a median AUC(0-∞) in ng·h/ml of the anti-proliferative agent generated by systemic delivery of the anti-proliferative agent to achieve the same tissue concentration at the injury site.

In some examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cmax) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cmin) of the direct factor IIa inhibitor generated by systemic delivery. In some examples, the blood concentration is smaller than a median minimum serum concentration (Cmin) of the direct factor IIa inhibitor generated by systemic delivery. In some examples, the Cmax is measured using one of plasma blood, serum blood, or whole blood. In other examples, the median Cmax is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC(0-∞)) in ng·h/ml which is smaller than a median (AUC(0-24) or AUC(0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC(0-24) or AUC(0-∞)) in ng·h/ml which is smaller than a median (AUC(0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC(0-24) or AUC (0-∞)) is measured using one of plasma blood, serum blood, or whole blood. In other examples, the median (AUC(0-24) or AUC(0-∞)) is 724 ng·h/ml, or 1437 ng·h/ml, or 2000 ng·h/ml, or 4000 ng·h/ml.

In some examples, local delivery of one or more of the active substances may reduce the time a patient needs to spend on oral medications and/or obviate the need for such medications entirely.

In some examples, the dose of each of the one or more active substances for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In further examples, the amount of each of the one or more active substances loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the fibrin/thrombus formation-inhibiting or fibrin/thrombus dissolution-promoting agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg.

In further examples, the concentration of each of the one or more active substances released from a temporary or non-temporary device, and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50, 100, 500 or 1000 μM. In certain examples, the concentration of each of the fibrin/thrombus formation-inhibiting or fibrin/thrombus dissolution-promoting agent(s) released from a temporary or non-temporary device, and optionally administered systemically in addition to locally, in blood or tissue at the site of injury or at an area adjacent thereto, and/or in blood or tissue adjacent to the device, independently is about 0.01 or 0.1 nM to about 1000 μM, or about 0.1 or 1 nM to about 500 μM, or about 1 or 10 nM to about 100 μM, or about 50 nM to about 50 μM, or about 10 or 100 nM to about 10 μM, or about 100 nM to about 1 μM, or about 1 μM to about 10 μM.

In still further examples, the concentration of each of the one or more active substances released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the fibrin/thrombus formation-inhibiting or fibrin/thrombus dissolution-promoting agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50) μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In additional examples, the concentration of each of the one or more active substances released from a temporary or non-temporary device (that may or may not cause an injury to a tissue, surface, vessel/lumen wall or other body part), and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50 or 100 μM, within about 1 day, 12 hr, 6 hr, 3 hr, 2 hr, 1 hr, 30 min., 15 min., 5 min, or 1 min, before, during and/or within about 1 day, 12 hr, 6 hr, 3 hr, 2 hr, 1 hr, 30 min., 15 min., 5 min, or 1 min, after delivery or deployment of the device and/or the injury. In further examples, the concentration of each of the one or more active substances released from a temporary or non-temporary device (that may or may not cause an injury to a tissue, surface, vessel/lumen wall or other body part), and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50 or 100 μg/gm tissue, within about 1 day, 12 hr, 6 hr, 3 hr, 2 hr, 1 hr. 30 min., 15 min., 5 min, or 1 min, before, during and/or within about 1 day, 12 hr, 6 hr, 3 hr, 2 hr, 1 hr, 30 min., 15 min., 5 min, or 1 min, after delivery or deployment of the device and/or the injury.

In some examples, the dose of each of the one or more optional other kinds of bioactive agents (e.g., anti-proliferative agents, anti-inflammatory agents, etc.) for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In additional examples, the amount of each of the one or more optional other kinds of bioactive agents (e.g., anti-proliferative agents, anti-inflammatory agents, etc.) loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the optional other kind(s) of bioactive agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg, or about 50 μg to about 200 μg.

In further examples, the concentration of each of the one or more optional other kinds of bioactive agents (e.g., anti-proliferative agents, anti-inflammatory agents, etc.) released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the optional other kind(s) of bioactive agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50) ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In some examples, the device contains the bioactive agent(s) in the body and/or on at least one surface of the device. In certain examples, the bioactive agent(s) are contained in one or more layers in the body and/or at the surface of the device.

In further examples, the bioactive agent(s) are contained in one or more coatings disposed over the body of the device. The coating(s) can be disposed over any desired portion(s) and any desired surface(s) of the body of the device. As a non-limiting example, for a tubular vascular device such as a stent, the coating(s) can be disposed over the luminal (lumen-facing) surface, the abluminal (tissue-facing) surface or the side surface(s) of the stent, or a combination thereof (e.g., all surfaces of the stent).

In additional examples, the device comprises the bioactive agent(s) in the body of the device and in one or more coatings disposed over the body of the device.

A temporary or non-temporary device can comprise openings in and/or on the body (including at the surface) of the device, and/or in one or more coatings disposed over the body structure of the device. Examples of openings include without limitation pores (including partial pores and through pores), holes (including partial holes and through holes), voids, recesses, pits, cavities, trenches, reservoirs and channels. In some examples, a temporary or non-temporary device contains one or more anti-coagulant, and optionally one or more other kinds of bioactive agents (e.g., anti-proliferative agents, anti-inflammatory agents, etc.) in openings in and/or on the body (including at the surface) of the device, and/or in one or more coatings disposed over the body of the device.

The device may comprise one or more coatings disposed over an exterior surface of a structure of the device, as described herein. In some embodiments, the coating(s) may comprise a homopolymer, a copolymer, a mixture of homopolymers, a mixture of copolymers, or a mixture of a homopolymer and a copolymer. In some examples, the coating(s) comprise a soft or hydrophilic, or a softer or more hydrophilic, polymeric material. In further examples, the coating(s) comprise a polymeric material and an additive (e.g., a monomer of the polymeric material) that softens the polymeric material.

In some examples, the device has a first coating that comprises a biodegradable or non-degradable polymeric material, or one or more bioactive agents, or both a biodegradable or non-degradable polymeric material and one or more bioactive agents. In further examples, the device has a second coating that comprises a biodegradable or non-degradable polymeric material, or one or more bioactive agents, or both a biodegradable or non-degradable polymeric material and one or more bioactive agents, wherein the second coating optionally is disposed over the first coating. In additional examples, the device has a third coating that comprises a biodegradable or non-degradable polymeric material, wherein the third coating is disposed over the first coating and/or the second coating. In some examples, the third coating serves as a top layer or coat or diffusion barrier that controls release of one or more bioactive agents from inner coating(s) and/or the body of the device.

In some examples, a bioactive agent that is intended to have an earlier or shorter time of action can be contained in an outer coating, on a surface uncovered by a coating, and/or in the body of the device closer to the surface, and a bioactive agent that is intended to have a later or longer time of action can be contained in an inner coating, in a coating covered by a barrier coating, on a surface covered by a coating, and/or in the body of the device farther from the surface. In further examples, a bioactive agent that is intended to have an earlier or shorter time of action is contained on a surface of the device, or contained in a coating on the device or in a layer of the body of the device which comprises a faster-degrading polymeric material, and a bioactive agent that is intended to have a later or longer time of action is contained within the device, or contained in a coating on the device or in a layer of the body of the device which comprises a slower-degrading or non-degrading polymeric material. In additional examples, a bioactive agent that is intended to have an earlier or shorter time of action is more soluble, and a bioactive agent that is intended to have a later or longer time of action is less soluble.

In certain examples, the concentration of a bioactive agent [e.g., anti-coagulant, anti-proliferative, etc.] in a coating comprising a polymeric material is at least about 10%, 20%, 30%, 40%, 50% or 60% by weight relative to the weight of the bioactive agent and the polymeric material.

In further examples, the thickness (e.g., average thickness) of each of the coating(s) independently is no more than about 20, 15, 10, 5, 3 or 1 micron.

In some examples, the coating(s) may comprise carrier material. Non-limiting examples of carrier materials include biodegradable polymeric materials, non-degradable polymeric materials, and other matrix materials.

In some examples, the carrier material may be porous. In certain examples, the porosity of each of the coating(s) of the carrier material may be within a range of about 10 nm to about 10 μm.

In some examples, the carrier material may be biodegradable. In certain examples, the carrier material may have a depredation rate within a range of about 1 month to about 36 months.

In some examples, the weight compositional ratio of the carrier material to the therapeutic composition of one or more bioactive agents may be within a range of about 1:5 to 3:2.

Non-limiting examples of polymeric materials that can compose the carrier material include polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide. D-lactide and D,L-lactide. The polymeric material may comprise a material selected from a group of non-degradable polymeric materials consisting of polyacrylates, polymethacrylates, poly(n-butyl methacrylate), poly(hydroxyethylmethacrylate), polyamides, nylons, nylon 12. Dacron. Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

Non-limiting examples of biodegradable polymeric materials that can compose the body of the device, a layer of the body, or a coating include polyesters, poly(α-hydroxyacids), polylactide, polyglycolide, poly(ε-caprolactone), polydioxanone, poly(hydroxyalkanoates), poly(hydroxypropionates), poly(3-hydroxypropionate), poly(hydroxy butyrates), poly(3-hydroxy butyrate), poly(4-hydroxy butyrate), poly(hydroxypentanoates), poly(3-hydroxypentanoate), poly(hydroxyvalerates), poly(3-hydroxyvalerate), poly(4-hydroxyvalerate), poly(hydroxyoctanoates), poly(3-hydroxyoctanoate), polysalicylate/polysalicylic acid, polycarbonates, poly(trimethylene carbonate), poly(ethylene carbonate), poly(propylene carbonate), tyrosine-derived polycarbonates. L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1.6HD), poly(DETOSU-t-CDM), polyurethanes, polyphosphazenes, polyamides, nylons, nylon 12, polyoxyethylated castor oil, poly(ethylene glycol), polyethylene oxide (PEO), polyvinylpyrrolidone, poly(L-lactide-co-D-lactide), ethylene-vinyl acetate, poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-8-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxy butyrate), poly(lactide-co-3-hydroxy butyrate), poly(lactide-co-4-hydroxy butyrate), poly(glycolide-co-hydroxy butyrate), poly(glycolide-co-3-hydroxy butyrate), poly(glycolide-co-4-hydroxy butyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxy butyrate-co-4-hydroxy butyrate), poly(hydroxy butyrate-co-hydroxyvalerate), poly(3-hydroxy butyrate-co-3-hydroxyvalerate), poly(3-hydroxy butyrate-co-4-hydroxyvalerate), poly(4-hydroxy butyrate-co-3-hydroxyvalerate), poly(4-hydroxy butyrate-co-4-hydroxyvalerate), poly(&-caprolactone-co-fumarate), poly(ε-caprolactone-co-propylene fumarate), poly(ester-co-ether), poly(lactide-co-ethylene glycol), poly(glycolide-co-ethylene glycol), poly(ε-caprolactone-co-ethylene glycol), poly(ester-co-amide), poly(DETOSU-1.6HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-8-caprolactone), poly(lactide-co-glycolide-co-trimethylene carbonate), poly(lactide-co-8-caprolactone-co-trimethylene carbonate), poly(glycolide-co-ε-caprolactone-co-trimethylene carbonate), poly(3-hydroxy butyrate-co-3-hydroxy valerate-co-4-hydroxy butyrate), poly(3-hydroxy butyrate-co-4-hydroxyvalerate-co-4-hydroxy butyrate), collagen, casein, polysaccharides, cellulose, cellulose esters, cellulose acetate, cellulose butyrate, cellulose acetate butyrate, cellulose propionate, chitin, chitosan, dextran, starch, modified starch, and copolymers and combinations thereof, wherein lactide includes L-lactide. D-lactide and D,L-lactide.

Examples of non-degradable polymeric materials that can compose the body of the device, a layer of the body, or a coating include without limitation polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene). Blend of polyvinylpyrrolidone, poly(hexylmethacrylate)-co-polyvinylpyrrolidone-co-poly vinyl acetate, and poly(n-butyl methacrylate)-co-poly(vinyl acetate). Poly(styrene-butylene styrene), poly(tyrosine-derived polycarbonate), polyamides, nylons, nylon 12, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate).), polyvinylpyridine block with poly methyl methacrylate (PMMA), poly N-(2-Hydroxypropyl) methacrylamide. Polyethylenimine (PEI), dextran, dextrin, chitosans, poly(L-lysine), and poly(aspartamides), polyamides. Polyethylene glycol (PEG). Silicones, poly(anhydride), poly ortho esters, polystyrene-b-polyvinylpyridine, poly(styrene)-poly (butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-polyethylene oxide) and copolymers and/or combinations thereof.

Non-limiting examples of corrodible metals and metal alloys that can compose the body of the device, a layer of the body, or a coating include cast ductile irons (e.g., 80-55-06 grade cast ductile iron), corrodible steels (e.g., AISI 1010) steel. AISI 1015 steel. AISI 1430 steel. AISI 5140 steel and AISI 8620 steel), melt-fusible metal alloys, bismuth-tin alloys (e.g., 40% bismuth-60% tin and 58% bismuth-42% tin), bismuth-tin-indium alloys, magnesium, magnesium alloys, tungsten alloys, zinc alloys, shape-memory metal alloys, and superelastic metal alloys. Examples of non-corrodible metals and metal alloys that can compose the body of the device, a layer of the body, or a coating include without limitation stainless steels (e.g., 316L stainless steel), cobalt-chromium alloys (e.g., L-605 and MP35N cobalt-chromium alloys), gold, molybdenum-rhenium alloys, nickel-titanium alloys, palladium, platinum, platinum-iridium alloys, tantalum, and alloys thereof.

In some examples, the device is coated. The coating layer may comprise a therapeutic agent and an additive. In some examples, the coating layer overlying an exterior surface of the exterior surface of the medical device consists essentially of the therapeutic agent and the additive.

In some examples, the additive is selected from PEG (polyethylene glycol), polyalkylene oxide, e.g., polyethylene oxide, polypropylene oxide, or a copolymer thereof (e.g., a polyethylene oxide-polypropylene oxide-polyethylene oxide copolymers), polyphenylene oxide, copolymers of PEG and polyalkylene oxide, poly(methoxyethyl methacrylate benzoate), poly(a methacryloyloxy one phosphatidylcholine), perfluorinated polyether, dextran or poly vinylpyrrolidone, poly(ethylene-vinyl acetate), polypeptides, water soluble surfactants, water soluble vitamins, and proteins, PEG fatty esters and alcohols, glycerol fatty esters, sorbitan fatty esters, PEGylation (PEG-drug conjugation), PEG glyceryl fatty esters, PEG sorbitan fatty esters, sugar fatty esters, PEG sugar esters, vitamins and derivatives, amino acids, multi amino acids and derivatives, peptides, polypeptides, oligomers, copolymers, block polymers, proteins, albumin, quaternary ammonium salts such as but not limited to benzalkonium chloride, benzethonium chloride, docecyl trimethyl ammonium bromide, sodium docecylsulfates, dialkyl methylbenzyl ammonium chloride, dialkylesters of sodium sulfonsuccinic acid, organic acids, salts and anhydrides and combinations thereof.

In another example, a factor Xa inhibitor, preferably Rivaroxaban, more preferably Apixaban released locally in combination with Argatroban from an implant to inhibit fibrin or clot formation.

In another example, a factor Xa inhibitor, preferably Rivaroxaban, more preferably Apixaban released locally in combination with Argatroban from an implant to inhibit fibrin, clot formation, and/or smooth muscle cell proliferation.

In another example, a factor Xa inhibitor, preferably Rivaroxaban, more preferably Apixaban released locally by an implant to inhibit clot formation.

In one example, a device delivery one or more drugs locally, wherein locally comprises delivering said one or more drugs to one or more of site specific location, to a vessel wall, adjacent to a vessel wall, in a body lumen, to a body organ, within a body organ, to the device surface in a body lumen, to a tissue, or to an injured tissue.

In another example, a factor Xa inhibitor, preferably Rivaroxaban, more preferably Apixaban is released locally in combination with an m-TOR inhibitor to inhibit fibrin formation or clot formation, or to inhibit fibrin formation or clot formation through 7 days, or to inhibit fibrin formation or clot formation through 28 days.

In another example, a third antiproliferative drug is configured to be released from the device in combination with Argatroban and Rivaroxaban or Apixaban, at similar dose and release rate or different dose and release rate. In a specific example, the anti-proliferative drug is sirolimus or its analogs (including deuterated analog), metabolites, or salts.

In one example, a device delivery one or more drugs locally, wherein locally comprises delivery of said one or more drugs to one or more of site specific location, adjacent to a vessel wall, to a vessel wall, in a body lumen, to the device surface in a body lumen, to a tissue, to an injured tissue, wherein the local concentration of the one or more dugs may be higher than in the systemic concentration of the one or more drugs.

In a preferred example, a device releasing factor Xa inhibitor in a body lumen wherein said device inhibits fibrin formation thereby inhibiting clot formation.

### EXPERIMENTAL EXAMPLES

### Example 1: Anti-Proliferative Activity of Apixaban Argatroban and Rapamycin Combination

Anti-proliferative activity of Apixaban, Argatroban, and Rapamycin was tested in Human Aortic SMC (HAoSMC, ATCC, PCS-100-012). Cell proliferation assay was done in 96-well format. Low passage cells were trypsinized and seeded in 96 well plates at a density of ˜4000 cells/well. The cells are allowed to attach overnight in a CO₂ incubator. Next day, the medium was removed and replaced with fresh complete medium containing various concentrations of the test compounds. The final concentration of vehicle (DMSO) in the test medium was 0.1%. After adding test compounds, the cells were incubated for 72 hours. Following this period, the medium was removed and then added fresh medium (100 μl) containing CellTiter Aqueous (1× concentration final) to the wells and incubated for 2 hours in the CO2 incubator. At the end of incubation measured fluorescence with a plate-reader. Controlled incubations with untreated cells and blank incubations containing only medium were included and tested similarly. Based on the cell viability assay the percentage inhibition of the cell proliferation was determined at the different concentrations of the drug tested.

The cell proliferation assay was performed with different concentrations of Apixaban and Argatroban when combined with Rapamycin. Following the cell proliferation assay as indicated earlier, the percent cell proliferation inhibition was determined, and the assay results plotted to determine the IC50.

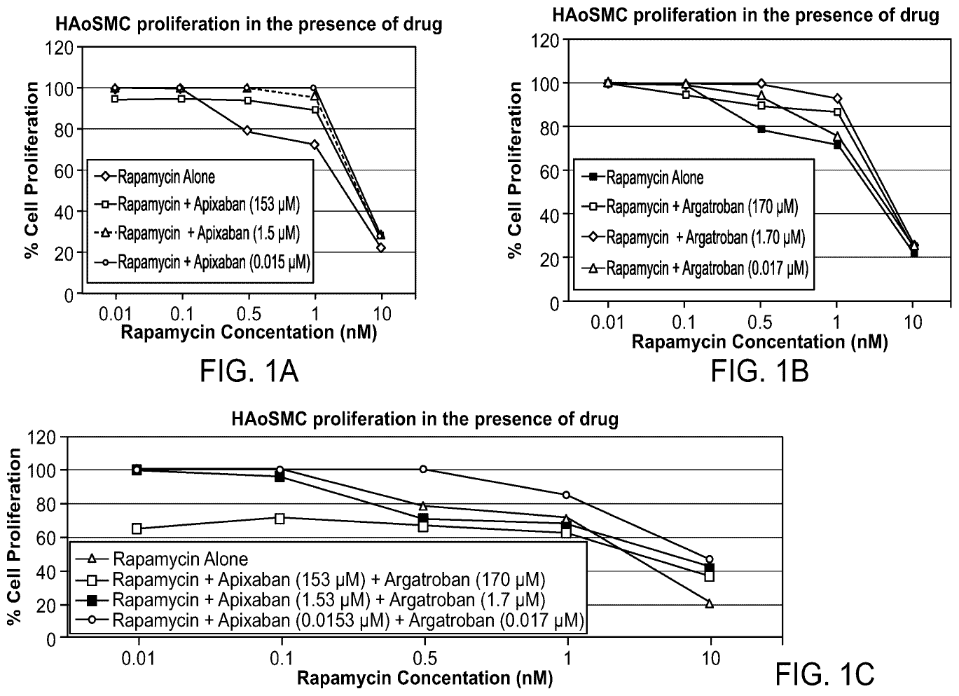

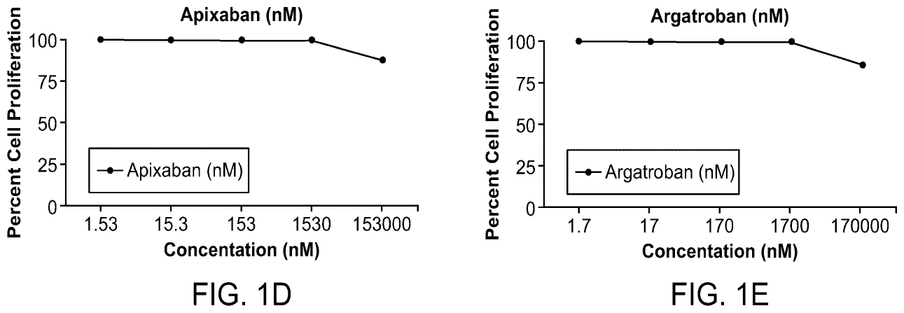

FIGS. 1A-IC show HAoSMC proliferation inhibition in the presence of different drug combinations. The data shows the combination of Apixaban, Argatroban surprisingly and unexpectedly enhanced the anti-proliferative effects of rapamycin on smooth muscle cell proliferation as measured by cell proliferation test when Apixaban and Argatroban were combined with rapamycin, i.e the combination of Apixaban, Argatroban, and rapamycin were more potent than rapamycin alone at inhibiting SMS proliferation.

FIGS. 1D and 1E show HAoSMC proliferation in presence of different concentrations of Apixaban or Argatroban. In order to determine if Apixaban or Argatroban independently had inhibitory effect on the proliferation of HAoSMC, a proliferation assay in the presence of either of these two drugs at different concentrations were tested as described earlier. Various concentration of Apixaban alone or Argatroban alone had small to no inhibition of HAoSMC proliferation was observed as shown in FIGS. 1D-1E.

### Example 2: Activated Clotting Time (ACT) Evaluation of Apixaban, Argatroban or a Combination of Apixaban and Argatroban

The activated clotting time (ACT) evaluation of anticoagulants was performed in calcium-reconstituted sheep blood and recorded employing the Hemochron® Response device.

The ACT measurements were made in citrated sheep blood. 1.9 ml of citrated sheep blood was added to a test tube containing an activator (Hemochrona@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of the drug as a single component. Selected drug combinations were then tested to evaluate for potential synergy in action between the two drugs.

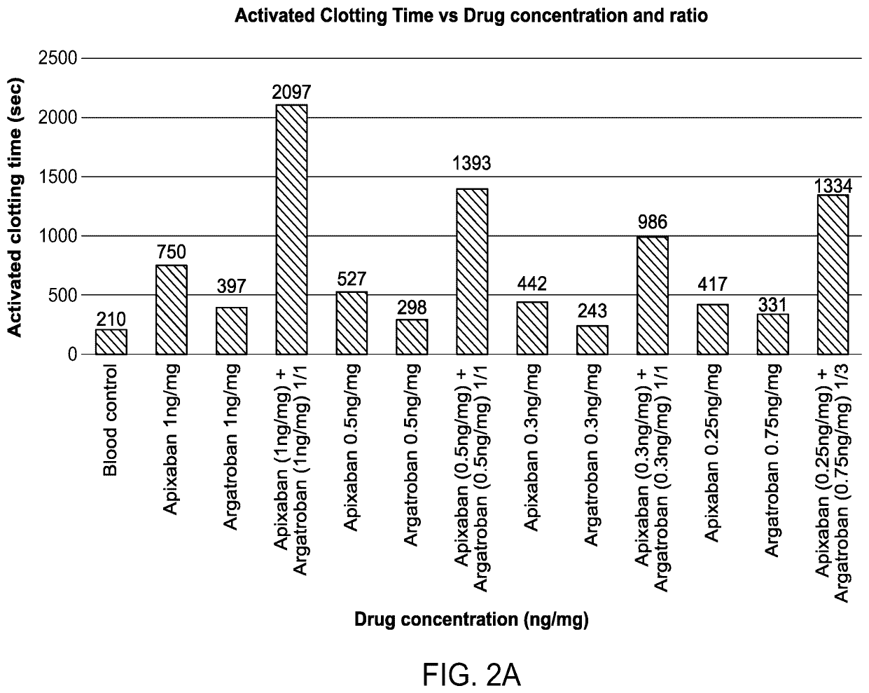

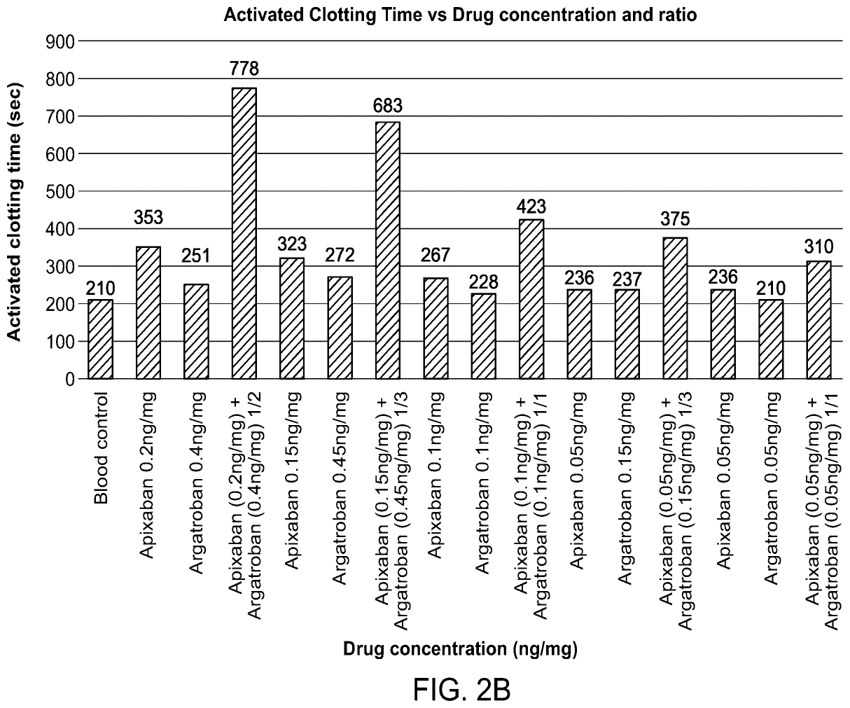

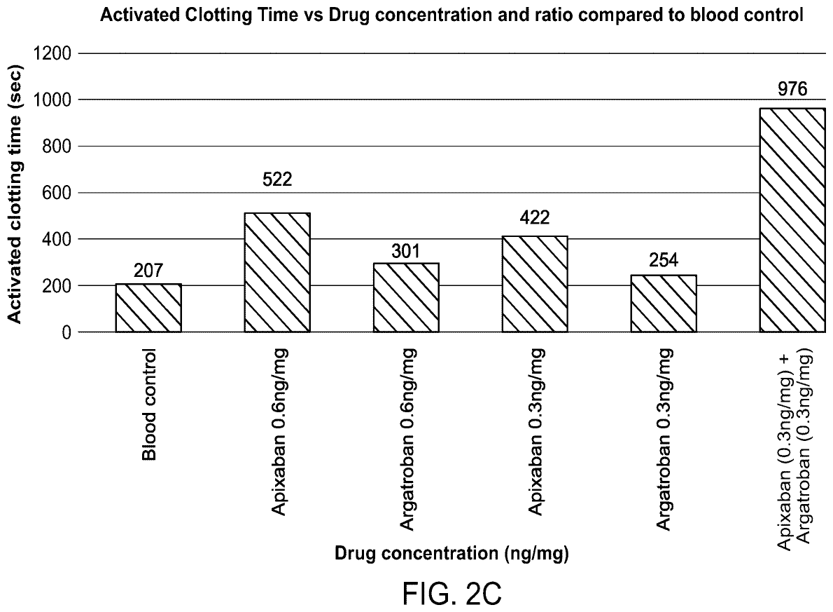

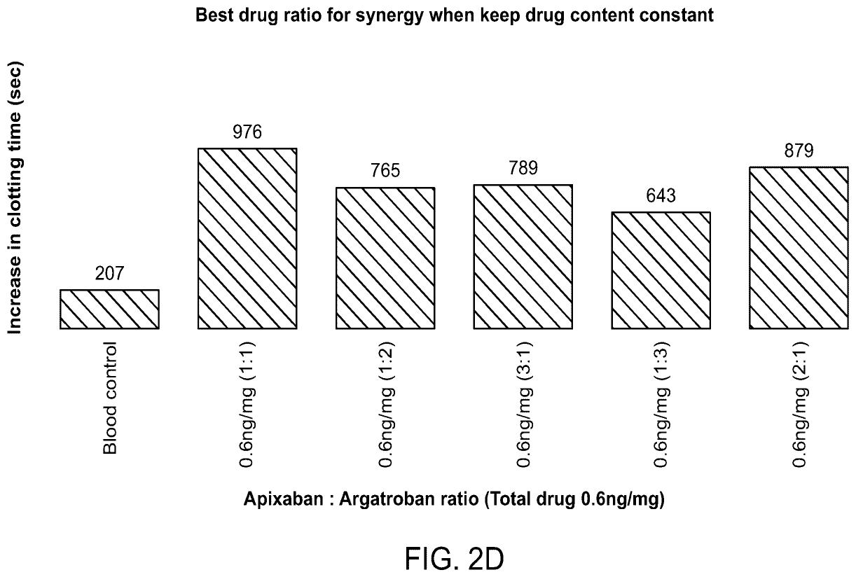

As shown in FIGS. 2A-2D, the clotting time was observed to be significantly extended or increased at a higher drug combination concentration. The Apixaban/Argatroban combination achieved ACT levels that were higher than the sum of the individual ACT values, indicating a synergistic effect between these drug combinations. This may be particularly important when delivering these drugs locally (adjacent to injured tissue) to inhibit clot formation. The figures are presented in ng/mg wherein the density of blood and tissue are approximately the same.

It was found, unexpectedly, that the combination drug concentrations of 0.025 ng/mg for each Apixaban and Argatroban drug extended the ACT by a larger time (as shown in FIG. 2B).

It was found, unexpectedly, that the combination drug concentrations of 0.3 ng/mg for each drug (0.6 ng/mg total) extended the ACT by a larger time (i.e., was more effective) than the ACT for each individual drug at 0.6 ng/mg concentration (ACT of 976 for the combination versus 522 for Apxaban versus 301 for Argatroban as shown in FIG. 2C).

FIG. 2C further shows, unexpectedly, that the combination drug concentrations of 0.3 ng/mg for each drug (0.6 ng/mg total) extended the ACT by a larger time (i.e., was more effective) than the ACT for the sum of each individual drug ACT at 0.3 ng/mg or at 0.6 ng/mg concentration. (ACT of 976 for the combination versus 676 (for the sum of individual drugs having 0.3 ng/mg concentrations).

It is important to note that drug tissue concentrations for factor Xa inhibitors like Rivaroxiban or Apixaban alone or in combination with factor IIa Argatrban to have sufficient tissue concentrations in the stented tissue segment and in the adjacent tissue segment to have therapeutic levels for each drug to be larger than 0.02 ng/mg, larger than 0.1 ng/mg, preferably larger than 0.2 ng/mg of tissue, preferably 0.3 ng/mg of tissue, more preferably larger than 1 ng/mg of tissue at or within 3 hours after implantation, or at or within 1 day after implantation, to inhibit clot formation.

### Example 3: Activated Clotting Time (ACT) Evaluation of EDTA or Analogues in Fresh Pig Blood

The activated clotting time (ACT) evaluation of EDTA or analogues was performed in fresh pig blood and recorded employing the Hemochron® Response device.

The ACT measurements were made in fresh pig blood. 1.0 ml of fresh pig blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of EDTA or analogues as a single component.

As shown in Table 1B, the clotting time was observed to be significantly extended or increased at a higher drug combination concentration. The EDTA or analogues achieved ACT levels that were higher than the control ACT values, indicating anti-coagulant effect. This may be particularly important when delivering EDTA or analogues locally (adjacent to injured tissue) to inhibit clot formation. The figures are presented in ng/mg wherein the density of blood and tissue are approximately the same.

TABLE 1B
Activated clotting time (ACT) versus EDTA or
analogues concentration in fresh pig blood

| | | | Increase in |
|---|---|---|---|
| | | | clotting |
| | | | time |
| | Final Drug | Clotting | compared |
| | Concentration | time | to control |
| Sample | (ng/mg) | (sec) | (sec) |
| Blood control | 0 | 154 | 0 |
| Dimercaptosuccinic | 200 | 151 | −3 |
| acid(DMSA) 200 μg/ml | | | |
| EDTA tetra acetoxymethyl | 500 | 158 | 4 |
| ester 500 μg/ml | | | |
| EDTA 75 μg/ml | 75 | 179 | 25 |
| EDTA 100 μg/ml | 100 | 247 | 93 |
| EDTA 150 μg/ml | 150 | 275 | 121 |
| EDTA 200 μg/ml | 200 | >500 | >346 |
| EDTA•4Na•xH2O 100 μg/ml | 100 | 204 | 50 |
| EDTA•4Na•xH2O 200 μg/ml | 200 | 275 | 121 |
| EDTA•4Na•xH2O 250 μg/ml | 250 | 329 | 175 |
| EDTA•4Na•xH2O 300 μg/ml | 300 | >500 | >346 |
| EDTA•2Na•2H2O 100 μg/ml | 100 | 191 | 37 |
| EDTA•2Na•2H2O 200 μg/ml | 200 | 264 | 110 |
| EDTA•2Na•2H2O 300 μg/ml | 300 | 374 | 220 |

As shown in Table 1B, EDTA at 200 ng/mg in fresh pig blood, had significant delayed blood coagulation compared to the blood control. The same trend was observed for EDTA tetra sodium salt and EDTA disodium salt at 300 ng/mg. It is important to note that drug tissue concentrations for EDTA or analogue to have sufficient tissue concentrations in the stented tissue segment and in the adjacent tissue segment to have therapeutic levels to be larger than 300 ng/mg of tissue at or within 3 hours after implantation, or at or within 1 day after implantation, to inhibit clot formation.

Based on the pH of EDTA or analogues in water and in 0.1M phosphate buffered saline as show in Table 2, EDTA disodium salt or dipotassium salt has a pH close to neutral pH in human body (6.42-6.92), whereas EDTA tetrasodium or trisodium has a very basic pH (>8.0) in human body. EDTA disodium or dipotassium is preferred in the examples.

TABLE 2
pH of 1 mg/ml EDTA or analogue in Water
or 0.1M pH 7.4 phosphate buffer.

| | | |
|---|---|---|
| Sample | pH | Temperature,° C. |

1 mg/ml in d-water

| | | |
|---|---|---|
| EDTA disodium | 6.42 | 21.8 |
| EDTA tetra sodium | 9.61 | 21.9 |
| EDTA tri sodium | 9.21 | 21.8 |
| EDTA dipotasium | 6.25 | 22 |

1 mg/ml in 0.01M pH 7.4 phosphate buffer

| | | |
|---|---|---|
| EDTA disodium | 6.92 | 22.3 |
| EDTA tetra sodium | 8.27 | 21.9 |
| EDTA tri sodium | 7.77 | 21.8 |
| EDTA dipotasium | 6.93 | 22.3 |

### Example 4: Activated Clotting Time (ACT) Evaluation of EDTA or Other Anti-Coagulant Compounds in Citrated Sheep Blood

The activated clotting time (ACT) evaluation of anticoagulants was performed in Calcium-reconstituted sheep blood and recorded employing the Hemochron® Response device.

The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of EDTA or other anti-coagulant compounds solution was then added into the test tube. The test tube was gently swirled so that the blood and EDTA or other anti-coagulant compounds was well-mixed. 0.1 ml of 0.3M calcium chloride was then added. The tube was gently shaken before being inserted into the Hemochron Response detector. The ACT read out was recorded and reported. The ACT of the control blood in the absence of any EDTA or other anti-coagulant compounds as first determined to establish a baseline. Then ACT was determined in the presence of different concentrations of EDTA or analogues as a single component.

As shown in Table 3, the clotting time was observed to be significantly extended or increased at a higher EDTA or other anti-coagulant compounds concentration. The EDTA or other anti-coagulant compounds achieved ACT levels that were higher than the control ACT values, indicating anti-coagulant effect. This may be particularly important when delivering EDTA or other anti-coagulant compounds locally (adjacent to injured tissue) to inhibit clot formation. The figures are presented in ng/mg wherein the density of blood and tissue are approximately the same.

TABLE 3
Activated clotting time (ACT) versus EDTA or other anti-
coagulant compounds concentration in citrated sheep blood

| | | | | Increase in |
|---|---|---|---|---|
| | | | | clotting |
| | | | | time |
| | Final | | Blood | compared to |
| | Concentration | Clotting | control | control |
| Sample | (ng/mg) | time (sec) | (sec) | (sec) |
| EDTA•2Na•2H2O with | 200 | >500 | 236 | >264 |
| Benzyldimethyltetradecylammonium chloride complex | | | | |
| 200 μg/ml | | | | |
| Benzyldimethyltetradecylammonium chloride 66.7 μg/ml | 67 | 316 | | 114 |
| Benzyldimethyltetradecylammonium chloride 100 μg/ml | 100 | 421 | | 219 |
| Benzyldimethyltetradecylammonium chloride 133.3 μg/ml | 133 | 983 | | 781 |
| Benzyldimethyltetradecylammonium chloride 166.7 μg/ml | 167 | 1370 | 202 | 1168 |
| Polyethylenimine, Linear, MW(250K), 3.3 μg/ml | 3 | 203 | | −14 |
| Polyethylenimine, Linear, MW(250K), 8.3 μg/ml | 8 | 188 | 217 | −29 |
| Polyethylenimine, Linear, MW(250K), 16.7 μg/ml | 17 | >500 | | >283 |
| Polyethylenimine, Linear, MW(250K), 33.3 μg/ml | 33 | >500 | | >283 |
| Polyethylenimine, Linear, MW(100K), 3.3 μg/ml | 3 | 198 | | −19 |
| Polyethylenimine, Linear, MW(100K), 8.3 μg/ml | 8 | 188 | | −29 |
| Polyethylenimine, Linear, MW(100K), 16.7 μg/ml | 17 | >500 | 217 | >283 |
| Polyethylenimine, Linear, MW(2.5K), 16.7 μg/ml | 17 | 303 | | >283 |
| Polyethylenimine, Linear, MW(2.5K), 25.0 μg/ml | 25 | 331 | | 129 |
| Polyethylenimine, Linear, MW(2.5K), 33.3 μg/ml | 33 | 668 | | 466 |
| Polyethylenimine, Linear, MW(2.5K), 66.7 μg/ml | 67 | 814 | | 612 |
| Polyethylenimine, Linear, MW(2.5K), 100 μg/ml | 100 | >793 | 202 | >591 |

It is important to note that drug tissue concentrations for EDTA or analogue to have sufficient tissue concentrations in the stented tissue segment and in the adjacent tissue segment to have therapeutic levels to be larger than 200 ng/mg of tissue with EDTA complex, or to be larger than 100 ng/mg of tissue with Benzyldimethyltetradecylammonium chloride, or to be larger than 33 ng/mg of tissue with linear Polyethylenimine or within 3 hours after implantation, or within 1 day after implantation, to inhibit clot formation.

### Example 5: Preparation of Anticoagulant EDTA Eluting Stents

Poly(L-lactide acid) polymer was dissolved into tetrahydrofuran (THF) at room temperature or heated 50° C., when needed, vortexed until the polymer had uniformly dissolved/dispersed. EDTA·4Na·xH₂O was dissolved into HPLC water at room temperature, stirred overnight until the drug was uniformly dissolved/dispersed.

Each polymer solution and each EDTA solution were combined together (EDTA to Poly(L-lactide acid) polymer by weight ratio was 7:3), according to the target EDTA dose. Top coated Poly(L-lactide) as needed per target release profile.

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 6: In Vitro Testing with Fast Release of Stent with EDTA in 0.01M Phosphate Buffered Saline Buffer

In-Vitro Elution evaluation of the EDTA coated stent above prepared was performed in 0.01M phosphate buffered saline at 37° C., water shaking bath then tested on High Performance Liquid Chromatography (HPLC). EDTA was separated from interfering peaks by a Carbon18 reverse phase column and quantified with an external EDTA calibration standard solution prepared at similar concentrations. The percentage of EDTA eluted was calculated during each time point.

TABLE 4
Cumulative percent release of EDTA percentage of release
profile in 0.01M phosphate buffered saline

| Time | 5 min | 10 min |
|---|---|---|
| 800 μg EDTA•4Na•xH2O | 99.8 | 100.0 |
| in Poly(L-Lactide acid) matrix with 200 μg | | |
| Poly(L-Lactide acid) topcoat | | |
| 800 μg EDTA•4Na•xH2O | 99.8 | 100.0 |
| in Poly(L-Lactide acid) matrix | | |
| EDTA•4Na•xH2O with 300 μg | | |
| Poly(L-Lactide acid) topcoat | | |

The data in Table 4 shows that about 99.8% EDTA tetra sodium eluted out about 5 minutes with top coated 200 μg Poly(L-lactide acid-co-glycolic acid) or with top coated 300 μg Poly(L-lactide acid-co-glycolic acid).

### Example 7: Preparation of Anticoagulant Tetra Acetoxymethyl Ester EDTA Eluting Stents

Poly(L-lactide acid-co-glycolic acid) or Poly(L-lactide acid) polymer was dissolved into dichloromethane (DCM) at room temperature, vortexed until the polymer had uniformly dissolved/dispersed. Tetra acetoxymethyl ester EDTA was dissolved into dichloromethane (DCM) at room temperature, stirred overnight until uniformly dissolved/dispersed.

Arm A was coated with 800 μg Tetra acetoxymethyl ester EDTA only; Arm B was coated with 800 μg Tetra acetoxymethyl ester EDTA solution then top coated 100 μg Nolvolimus in poly(L-lactide acid-co-glycolic acid) matrix (drug to polymer ratio of 2:3); Arm C was coated with 300 μg Tetra acetoxymethyl ester EDTA in 600 μg poly(L-lactide acid) matrix.

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 8: In Vitro Testing of Controlled Release of Stent with Tetra Acetoxymethyl Ester EDTA in 0.01M Phosphate Buffered Saline

In-Vitro Elution evaluation of Tetra acetoxymethyl ester EDTA coated stent was performed in 0.01M phosphate buffered saline at 37° C., water shaking bath then tested on High Performance Liquid Chromatography (HPLC). Tetra acetoxymethyl ester EDTA was separated from interfering peaks by a Carbon18 reverse phase column and quantified with an external tetra acetoxymethyl ester EDTA calibration standard solution prepared at similar concentrations. The percentage of tetra acetoxymethyl ester EDTA eluted was calculated during each time point.

TABLE 5
Cumulative percent release of tetra acetoxymethyl ester EDTA percentage
of release profile in 0.01M phosphate buffered saline

| | | | | Left after 15 |
|---|---|---|---|---|
| Time | 5 min | 10 min | 15 min | min |
| Arm A: 800 μg Tetra acetoxymethyl ester EDTA only | 71.4 | 76.0 | 77.0 | 23.0 |
| Arm B: 800 μg Tetra acetoxymethyl ester EDTA with top | 46.0 | 68.3 | 77.4 | 22.6 |
| coated with 100 μg Novolimus in 175 μg Poly(L-lactide | | | | |
| acid-co-glycolic acid) matrix | | | | |
| Arm C: 300 μg Tetra acetoxymethyl ester EDTA in 600 μg | 6.8 | 14.0 | 14.0 | 86.0 |
| Poly(L-lactide acid) matrix | | | | |

The data in Table 5 shows that with top coated 175 μg of Poly(L-lactide acid-co-glycolic acid) and 100 μg of Novolimus, the release rate of Tetra acetoxymethyl ester EDTA was extended to about 10 minutes to 15 minutes (Arm B). With tetra acetoxymethyl ester EDTA and poly(L-lactide acid) matrix (Arm C), the release rate was further slowdown beyond 15 minutes. Arm B released around 200 μg of EDTA between 5 minutes to 10 minutes in the studied dose, having an effective therapeutic level to inhibit clot formation.

### Example 9: Preparation of Anticoagulant EDTA Complex Eluting Stents

EDTA complex was made as below: 3 moles of EDTA·2Na. 2H2O with 1 mole of Benzyldimethyltetradecylammonium chloride was reacted in room temperature overnight. The complex was purified by removing sodium chloride and excessive EDTA·2Na·2H2O then vacuum dried.

Poly(L-lactide acid-co-glycolic acid) polymer was dissolved into dichloromethane (DCM) at room temperature and vortexed until the polymer had uniformly dissolved/dispersed. EDTA·2Na·2H2O with benzyldimethyltetradecylammonium complex was dissolved into ethanol at room temperature and stirred until uniformly dissolved/dispersed.

Each polymer solution and each EDTA complex were combined together (EDTA to Poly(L-lactide acid-co-glycolic acid) polymer by weight ratio was 3:2), according to the target EDTA dose as shown in Arm D, Arm E and Arm F. Top coated Poly(L-lactide acid-co-glycolic acid) as needed per release profile.

A microprocessor-controlled ultrasonic sprayer was used to coat each of the stents' 14 mm length uniformly with each of the excipient/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 10: In Vitro Testing of Controlled Release of Stent with EDTA Complex in 0.01M Phosphate Buffered Saline

In-Vitro Elution evaluation of the EDTA complex coated stent was performed in 0.01M phosphate buffered saline at 37° C., water shaking bath then tested on High Performance Liquid Chromatography (HPLC). EDTA disodium was separated from interfering peaks by a Carbon18 reverse phase column and quantified with an external EDTA disodium calibration standard solution prepared at similar concentrations. The percentage of EDTA disodium eluted was calculated during each time point as listed below.

TABLE 6
Cumulative percent release of EDTA disodium percentage of release profile in 0.01M phosphate buffered saline

| | | | | | | | | | Total |
|---|---|---|---|---|---|---|---|---|---|
| | | | | | | | | | eluted + |
| | | 10 | 15 | | | | | | 24 hrs |
| Time | 2 min | min | min | 20 min | 25 min | 30 min | 3 hrs | 24 hrs | left |
| Arm D: 1200 μg EDTA.2Na•2H₂O with | 72.0 | 75.2 | 76.9 | 79.1 | 80.4 | 83.0 | 87.2 | 92.0 | 99.9 |
| benzyldimethyltetradecylammonium complex in | | | | | | | | | |
| 800 μg Poly(L-lactide acid-co-glycolic acid) | | | | | | | | | |
| matrix | | | | | | | | | |
| Arm E: 1200 μg EDTA.2Na•2H₂O with | 10.2 | 16.7 | 23.8 | 31.1 | 37.3 | 43.0 | 88.6 | 97.9 | 99.9 |
| benzyldimethyltetradecylammonium complex in | | | | | | | | | |
| 800 μg Poly(L-lactide acid-co-glycolic acid) | | | | | | | | | |
| matrix with 200 μg Poly(L-lactide acid-co- | | | | | | | | | |
| glycolic acid) topcoat | | | | | | | | | |
| Arm F: 1200 μg EDTA.2Na•2H₂O with | 8.6 | 12.4 | 15.4 | N/A | N/A | 21.2 | 45.7 | 62.1 | 100 |
| benzyldimethyltetradecylammonium complex in | | | | | | | | | |
| 800 μg Poly(L-lactide acid-co-glycolic acid) | | | | | | | | | |
| matrix with 400 μg Poly(L-lactide acid-co- | | | | | | | | | |
| glycolic acid) topcoat | | | | | | | | | |

The data in Table 6 shows that about 72% EDTA disodium eluted out from EDTA complex at 2 minutes without topcoat (Arm D); extended release of EDTA disodium is achieved with 200 μg topcoat Poly(L-lactide acid-co-glycolic acid) (Arm E) to 3 hours (about 88.6% eluted out up to 3 hours), with increased topcoat of 400 μg of Poly(L-lactide acid-co-glycolic acid) (Arm E), the release extended beyond 24 hours (about 62.1% eluted out up to 24 hours). Arm E and Arm F released around 300 μg to 550 μg of EDTA between 30 minutes to 3 hours in the studied dose, indicating an effective therapeutic level to inhibit local clot formation.

### Example 11: Preparation of Metal Stents with Cationic Anti-Coagulation Enhancer Benzyldimethyltetradecyl Ammonium Chloride

Poly(L-lactide acid-co-glycolic acid) polymer was dissolved into dichloromethane (DCM) at room temperature and vortexed until the polymer had uniformly dissolved/dispersed. Cationic anti-coagulation enhancers Benzyl dimethyltetradecyl ammonium chloride was dissolved into ethanol at room temperature and stirred overnight until uniformly dissolved/dispersed.

Each polymer solution and each cationic anti-coagulation enhancers solution were combined together (cationic anti-coagulation enhancers to Poly(L-lactide acid-co-glycolic acid) polymer by weight ratio 7:10), according to the target cationic anti-coagulation enhancers released dose. Top coated Poly(L-lactide acid-co-glycolic acid) as needed per target release profile.

A microprocessor-controlled ultrasonic sprayer was used to coat each of the stents' 14 mm length uniformly with each of the cationic anti-coagulation enhancer/polymer matrix solutions. After coating, the stents were placed in a vacuum chamber to remove the solvent. The stents were then mounted on balloon catheters and crimped. The catheters were then inserted in coils and packaged. The pouches were sterilized.

### Example 12: In Vitro Testing of Extended Release of Stent Coated with Cationic Anti-Coagulation Enhancer Benzyl Dimethyltetradecyl Ammonium Chloride with Poly(L-Lactide Acid-Co-Glycolic Acid) Polymer

In-Vitro Elution evaluation of the stent coated with cationic anti-coagulation enhancer Benzyl dimethyl tetradecyl ammonium chloride and Poly(L-lactide acid-co-glycolic acid) was performed in 0.01M phosphate buffered saline at 37° C., water shaking bath then tested on High Performance Liquid Chromatography (HPLC). Benzyl dimethyl tetradecyl ammonium chloride was separated from interfering peaks by a Carbon18 reverse phase column and quantified with an external Benzyl dimethyl tetradecyl ammonium chloride calibration standard solution prepared at similar concentrations. The percentage of cationic anti-coagulation enhancer eluted was calculated during each time point as listed below.

TABLE 7
Cumulative percent release of cationic anti-coagulation enhancer Benzyl dimethyl tetradecyl
ammonium chloride percentage of release profile in 0.01M phosphate buffered saline.

| | | | | | | | | | Total eluted + 24 hrs |
|---|---|---|---|---|---|---|---|---|---|
| Time | 2 min | 10 min | 15 min | 20 min | 25 min | 30 min | 3 hrs | 24 hrs | left |
| Arm G: 1500 μg benzyl dimethyl | 20.3 | 30 | 34.6 | 40.4 | 44.9 | 49.1 | 92.0 | 97.8 | 98.3 |
| tetradecyl ammonium complex in | | | | | | | | | |
| 643 μg Poly(L-lactide acid-co- | | | | | | | | | |
| glycolic acid) matrix with 200 μg | | | | | | | | | |
| Poly(L-lactide acid-co-glycolic acid) | | | | | | | | | |
| topcoat | | | | | | | | | |
| Arm H: 1500 μg benzyl dimethyl | 21.5 | 28.7 | 31.9 | 35.3 | 38.1 | 41.2 | 83.9 | 97.1 | 100 |
| tetradecyl ammonium complex in | | | | | | | | | |
| 643 μg Poly(L-lactide acid-co- | | | | | | | | | |
| glycolic acid) matrix with 400 μg | | | | | | | | | |
| Poly(L-lactide acid-co-glycolic acid) | | | | | | | | | |
| topcoat | | | | | | | | | |

The data in Table 7 shows that extended release of Benzyl dimethyl tetradecyl ammonium chloride is achieved with 200 μg top coated poly(L-lactide acid-co-glycolic acid) (Arm G) to 3 hours (about 92.0% eluted out up to 3 hours), with increased topcoat (400 μg of Poly(L-lactide acid-co-glycolic acid) Arm H), the release extended to 24 hours (about 97.1% eluted out up to 24 hours). Arm G and Arm H released about 600 μg to 650 μg of EDTA between 30 minutes to 3 hours in the studied dose, indicating an effective therapeutic level to inhibit local clot formation.

### Example 13: Preparation of Novolimus and Cationic Anti-Coagulation Enhancer Linear Polyethylenimine Coated Stent

Basecoat solution was prepared as below: Poly(L-lactide acid-co-glycolic acid) polymer was dissolved into dichloromethane (DCM) at room temperature, vortexed until the polymer had uniformly dissolved/dispersed. Novolimus was dissolved into dichloromethane (DCM) at room temperature and stir overnight until uniformly dissolved/dispersed. The polymer solution and Novolimus solution were combined together (Poly(L-lactide acid-co-glycolic acid) to Novolimus by weight ratio was 3:2), according to the target Novolimus dose of 100 μg for a 14 mm stent.

Topcoat solution was prepared as below: Poly(L-lactide acid-co-glycolic acid) polymer was dissolved into dichloromethane (DCM) at room temperature and vortexed until the polymer had uniformly dissolved/dispersed. Cationic anti-coagulation enhancers Linear Polyethylenimine (MW=2,500) was dissolved into ethanol at room temperature and stir overnight until uniformly dissolved/dispersed. Each polymer solution and each cationic anti-coagulation enhancers solution were combined together according to the target cationic anti-coagulation enhancers and polymer weight.

A microprocessor-controlled ultrasonic sprayer was used to coat each of the stents' 14 mm length uniformly with each of the base coat and topcoat matrix solutions. After coating, the stents were placed in a vacuum chamber to remove the solvent. The stents were then mounted on balloon catheters and crimped. The catheters were then inserted in coils and packaged. The pouches were sterilized.

### Example 14: In Vitro Testing of Extended Release of Stent with Novolimus and Cationic Anti-Coagulation Enhancer Linear Polyethylenimine

In-Vitro Elution evaluation of the stent with Novolimus and cationic anti-coagulation enhancer linear polyethylenimine was performed in 0.01M phosphate buffered saline at 37° C. water shaking bath then tested on High Performance Liquid Chromatography (HPLC). Polyethylenimine was separated from interfering peaks by a Carbon18 reverse phase column and quantified with an external Benzyl dimethyl tetradecyl ammonium chloride calibration standard solution prepared at similar concentrations. The percentage of cationic anti-coagulation enhancer eluted was calculated during each time point as listed below.

TABLE 8
Cumulative percent release of cationic anti-coagulation enhancer polyethylenimine
percentage of release profile in 0.01M phosphate buffered saline

| Time | 2 min | 5 min | 30 min | 2 hrs | 8 hrs | 24 hrs |
|---|---|---|---|---|---|---|
| Arm I: 560 μg linear polyethylenimine with 100 μg | 13.8 | 73.5 | 81.5 | 90.2 | 97.6 | 100.0 |
| Novolimus in 400 μg Poly(L-lactide acid-co-glycolic | | | | | | |
| acid) matrix | | | | | | |
| Arm J: Base coated 100 μg Novolimus in Poly(L- | 6.6 | 81.6 | 87.5 | 93.3 | 98.5 | 100.0 |
| lactide acid-co-glycolic acid) matrix and top coated | | | | | | |
| 800 μg linear polyethylenimine with 200 μg Poly(L- | | | | | | |
| lactide acid-co-glycolic acid) | | | | | | |
| Arm K: Base coated 100 μg Novolimus in Poly(L- | 6.9 | 82.4 | 87.6 | 93.6 | 98.6 | 100.0 |
| lactide acid-co-glycolic acid) matrix and top coated | | | | | | |
| 800 μg linear polyethylenimine with 400 μg Poly(L- | | | | | | |
| lactide acid-co-glycolic acid) | | | | | | |

The data in Table 8 shows that the release of polyethylenimine was similar with polyethylenimine and poly(L-lactide acid-co-glycolic acid) matrix either with one layer or only in the top coat. Arm J and Arm K released around 600 μg of polyethylenimine between 2 minutes to 5 minutes in the studied dose, which is more than an effective dose of 33 μg/ml of Linear Polyethylenimine, indicating an effective therapeutic level to inhibit local clot formation.

### Example 15: Preparation of Anticoagulant (Rivaroxaban, Argatroban, and Dalteparin) Eluting Stents

Poly(n-butyl methacrylate) polymer was dissolved into dichloromethane (tetrahydrofuran (THF) was used for Dalteparin) at room temperature and vortexed until the polymer had uniformly dissolved/dispersed. Rivaroxaban was dissolved into dichloromethane at room temperature and vortexed until the drug was uniformly dissolved/dispersed. Argatroban (and Argatroban in combination with Rivaroxaban) was dissolved in Methanol and dichloromethane and vortexed at room temperature until the drug was uniformly dispersed/dissolved. Dalteparin was dissolved in water and THE until fully dissolved.

Each polymer solution and each drug solution were combined together (rivaroxaban to poly(n-butyl methacrylate) by weight ratio was 6:1), (Argatroban to poly(n-butyl methacrylate) by weight ratio was 3:4), (Dalteparin to poly(n-butyl methacrylate) by weight ratio was 2:3), and (Rivaroxaban in combination with Argatroban to Poly(n-butyl methacrylate) weight ratio was 3:2:2) according to the target drug dose of 150 μg for each drug (and 100 μg each for the rivaroxaban and Argatroban combination).

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized. The bare metal control stents were the same as the other stents without a drug or polymer coating.

### Example 16: In Vivo Testing of Drug Eluting Stent with Different Drugs

The drug eluting stent systems containing different anticoagulants prepared as described in Example 11 were evaluated at 3 hours, 6 hours, 1 day, 3 days, 6 days, 7 days, or 28 days following implantation in a porcine coronary artery model.

The porcine model was chosen as this model has been used extensively for stent and angioplasty studies resulting in a large volume of data on the vascular response properties and its correlation to human vascular response (Schwartz et al, Circulation. 2002; 106:1867 1873). The animals were housed and cared for in accordance with the Guide for the Care and Use of Laboratory Animals as established by the National Research Council.

After induction of anesthesia, the left or right femoral artery was accessed using standard techniques and an arterial sheath was introduced and advanced into the artery. Vessel angiography was performed under fluoroscopic guidance, a 7 Fr, guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately size stent was advanced to the deployment site. The balloon was inflated at a steady rate to a pressure sufficient to achieve a balloon to artery ratio of approximately 1.1 to 1.0.

Follow up angiography was performed at the designated timepoint for each of the animals. Late lumen loss (LLL) can be expressed as:

LLL=Post-stent minimum lumen diameter−Final minimum lumen diameter

The LLL is an indicator of the amount smooth muscle cell (SMC) proliferation or inhibition. It is used to measure efficacy between drugs for SMC proliferation inhibition. The smaller the LLL, the better the efficacy of the drug.

Stented portions of coronary arteries were embedded in methyl methacrylate (MMA), then divided into a target of at least three blocks of approximately similar lengths for histology evaluation. Quantitative histopathological evaluation of stented artery sections was then performed and scored as indicated. For Fibrin formation, scores ranged from 0 to 3, with a score of 0 indicating absent or rare minimal spotting around struts of the stent, a score of 1 indicating the presence of fibrin in small amounts localized only around the struts, a score of 2 indicating the moderately abundant or denser presence of fibrin around and extending beyond the struts, and a score of 3 indicating the presence of abundant and dense fibrin and/or bridging of the fibrin between the struts. The mean score was calculated and reported. The mean of each section was then averaged to provide a mean fibrin score per stent. The smaller the fibrin score, the better efficacy.

The percentage and/or amount of each anticoagulant drug for or by each time point indicated were analyzed for each stent from the different devices in the example and the average drug tissue concentration reported.

Tissue concentrations and the amount of drug released from the stents were measured using stents implanted in porcine arteries for the drugs as indicated. The arteries at the designated time point were excised and a length of stented artery spanning from 5 mm proximal to the stented segment to 5 mm distal to the stented segment was cut. The stented artery was cut longitudinally with surgical scissors. The stents were separated from the tissue. The tissue content of each drug was analyzed using liquid chromatography mass spectroscopy (LCMS) and reported as a mean for each of the timepoint indicated. For drug remaining on each stent, each drug was extracted from the stent, measured using HPLC, and reported as a mean for each of the timepoint indicated as drug released or drug remaining on a stent (where drug remaining is equal to 100% minus the percentage of drug released).

TABLE 9
Fibrin score, PK, and late lumen loss of Rivaroxaban, Argatroban, and Dalteparin
(low molecular weight heparin) released from 14 mm stents at day 7.

| | | Cumulative Percent | | |
|---|---|---|---|---|
| Stent coating information | Fibrin score | Release of drug by | Tissue concentration at | LLL at |
| (n = 3 for each arm) | at day 7 | day 7 | day 7 (ng/mg) | day 7 |
| 150 μg Rivaroxaban & Poly(n-butyl | 0.72 ± 0.12 | 99.6% | 3.9 ± 0.6 | 0.31 ± 0.20 |
| methacrylate) matrix coated stent | | | | |
| 150 μg Argatroban & Poly(n-butyl | 1.25 ± 0.87 | 47.7% | 5.7 ± 1.8 | 0.34 ± 0.20 |
| methacrylate) matrix coated stent | | | | |
| 100 μg Argatroban and 100 μg | 0.80 ± 0.39 | 70.9% for | 48.1 ± 43.6 for | 0.04 ± 0.06 |
| Rivaroxaban in Poly(n-butyl | | Rivaroxaban | rivaroxaban | |
| methacrylate) matrix coated stent | | 96.5% for Argatroban | 8.9 ± 6.3 for Argatroban | |
| 150 μg Dalteparin(low molecular | 1.50 ± 0.82 | 98.3% | Not tested | 0.23 ± 0.20 |
| weight heparin) in Poly(n-butyl | | | | |
| methacrylate) matrix coated stent | | | | |
| Bare metal control stent (BMS) | 1.02 ± 0.35 | N/A | N/A | 0.17 ± 0.22 |

As shown in Table 9, Rivaroxaban released from stents was effective at inhibiting fibrin formation compared to bare metal control stents, while Argatroban released from stents or Dalteparin released from stents were not effective at inhibiting fibrin formation compared to bare metal control stents.

As shown in Table 9, Rivaroxaban, Argatroban, or Dalteparin released from stents as single agents had larger LLLs compared to control and thus were not effective at inhibiting smooth muscle cell proliferation compared to bare metal control stents.

As shown in Table 9, the combination of Rivaroxaban and Argatroban released from stents had a smaller LLL compared to control and thus was effective at inhibiting smooth muscle cell proliferation compared to bare metal control stents. Furthermore, the combination of Rivaroxaban and Argatroban released from stents was effective at inhibiting fibrin formation compared to bare metal control stents.

As shown in Table 9, Rivaroxaban released from stents at a dose of at least 150 μg within 7 days from implant (or from vessel injury) was effective at inhibiting fibrin formation.

As shown in Table 9, Rivaroxaban released from stents at a dose of at least 1.8 μg/mm2 within 7 days from implant (or from vessel injury) was effective at inhibiting fibrin formation.

As shown in Table 9, Rivaroxaban released from stents at a dose of at least 10.7 μg/mm of stent length within 7 days from implant (or from vessel injury) was effective at inhibiting fibrin formation.

As shown in Table 9, Rivaroxaban dose of at least 150 μg, or of at least 1.8 μg/mm2, or at least 10.7 μg/mm of device length, released from a stents device at a release rate of about 99.6% within 7 days from implant (or from time of injury) was effective at inhibiting fibrin formation.

As shown in Table 9. Rivaroxaban released from stents at a release rate of at least 70.9% when combined with Argatroban at a release rate of at least 96.9% within 7 days from implant (or from time of injury) was effective at inhibiting fibrin formation.

Table 9 shows Rivaroxaban released from a stent at a dose of at least 100 μg, or at a dose of at least 1.2 μg/mm2, or at a dose of at least 7.14 μg/mm of stent length, at a release rate of at least 70.9% within 7 days when combined with Argatroban released from a stent at a dose of at least 100 μg, or at a dose of at least 1.2 μg/mm2, or at a dose of at least 7.14 μg/mm of stent length, at a release rate of at least 96.9% within 7 days from implant (or from time of injury) was effective at inhibiting fibrin formation.

### Example 17: Preparation of Rivaroxaban and m-TOR Inhibitor Releasing Stent

Base coat of Novolimus (m-TOR inhibitor) and Poly(n-butyl methacrylate) matrix: Poly(n-butyl methacrylate) polymer was dissolved into dichloromethane at room temperature and vortex until the polymer had uniformly dissolved/dispersed. Novolimus was placed in another vial and dissolved in dichloromethane at room temperature until uniformly dissolved or dispersed. The polymer solution and drug solutions were mixed together and coated as a matrix (the drug to polymer weight ratio was 2:3 by weight).

Top coat of Rivaroxaban and poly(n-butyl methacrylate) matrix: Poly(n-butyl methacrylate) polymer was dissolved in dichloromethane at room temperature and vortex until the polymer had uniformly dissolved/dispersed. Rivaroxaban was dissolved into dichloromethane at room temperature and vortex until the drug was uniformly dissolved/dispersed. Each polymer solution and each drug solutions were mixed together as a matrix (rivaroxaban to poly(n-butyl methacrylate) by weight ratio was 6:1 for the rivaroxaban fast formulation without m-TOR). Rivaroxaban to poly(n-butyl methacrylate) ratio was 4:1 for the fast release formulation with m-TOR base coat matrix, and 2:1 for the slow release formulation with m-TOR base coat matrix according to the target drug dose of 100 μg Rivaroxaban and 25 μg poly(n-butyl methacrylate) for fast release formulation, and 100 μg Rivaroxaban and 50 μg poly(n-butyl methacrylate) for the slow release formulation.

A microprocessor-controlled ultrasonic sprayer was used to coat each of the stents' 14 mm length uniformly with each of the drug/polymer matrix solution with the base coat matrix first, placing the stents in vacuum chamber to remove the solvent, followed by the top coat matrix. The stents were placed in a vacuum chamber again to remove the solvents. The stents were then mounted on balloon catheters and crimped. The catheters were then inserted in coils and packaged. The pouches were sterilized. The Novolimus (m-TOR inhibitor) stents controls (DES) consisted of only the base coat drug/polymer matrix, without the top coat drug/polymer matrix, otherwise being the same as the other stents. The bare metal control stents (BMS) were the same as the other stents without a drug or polymer coating.

TABLE 10
Fibrin score and PK of Rivaroxaban releasing 14 mm stents at day 7 and at day 28.

| | 7 days n = 5 | | | 28 days n = 5 | | |
|---|---|---|---|---|---|---|
| | | Cumulative | | | Cumulative | |
| | | Percent | Tissue | | Percent | Tissue |
| Stent coating information n = 5 for | Fibrin | Release of | concentration | Fibrin | Release of | concentration |
| each arm for each time point | score | drug | (ng/mg) | score | drug | (ng/mg) |
| 25 μg Poly(n-butyl methacrylate) & | 0.79 ± | 99.7% | 3.95 ± | 0.07 ± | 100% | 3.93 ± |
| 150 μg Rivaroxaban matrix | 0.14 | | 1.57 | 0.05 | | 2.43 |
| 66 μg Novolimus (m-TOR inhibitor) | 1.16 ± | 88.9% | 6.18 ± | 1.06 ± | 92.5% | 14.61 ± |
| and 100 μg Poly(n-butyl | 0.60 | | 5.37 | 0.46 | | 17.68 |
| methacrylate) matrix as a base coat | | | | | | |
| then 100 μg Rivaroxaban and 25 μg | | | | | | |
| Poly(n-butyl methacrylate) matrix as | | | | | | |
| a top coat | | | | | | |
| 66 μg Novolimus (m-TOR inhibitor) | 1.18 ± | 68.1% | 8.56 ± 3.56 | 1.49 ± | 72.7% | 15.23 ± |
| and 100 μg Poly(n-butyl | 0.53 | | | 0.37 | | 8.12 |
| methacrylate) matrix as a base coat | | | | | | |
| then 100 μg Rivaroxaban and 50 μg | | | | | | |
| Poly(n-butyl methacrylate) matrix as | | | | | | |
| a top coat | | | | | | |
| 66 μg Novolimus (m-TOR inhibitor) | 1.51 ± | — | — | 1.37 ± | — | — |
| and 100 μg Poly(n-butyl | 0.71 | | | 0.44 | | |
| methacrylate) matrix (DES) control | | | | | | |
| Bare metal control (BMS) | 1.35 ± | — | — | — | — | — |
| | 0.41 | | | | | |

As shown in Table 10, Rivaroxaban released from a stent was effective at inhibiting fibrin formation compared to control at 7 days and at 28 days when it was released at a faster rate and/or at a larger dose whether it was released alone or in combination with an m-TOR inhibitor, while it (Rivaroxaban) was not effective at inhibiting fibrin formation compared to control at 28 days when it was released at a slower rate and/or at a smaller dose.

As shown in Table 10, Rivaroxaban released from a stent was more effective at inhibiting fibrin formation compared to control at 7 days and at 28 days when it was released at a rate of at least 99.7% within 7 days and/or when a dose of at least 150 μg, or a dose of at least 2.0 μg/mm2, or a dose of at least 10.7 μg/mm of stent length, was released within 7 days from vessel injury (or from implantation).

As shown in Table 10, Rivaroxaban released from a stent was more effective at inhibiting fibrin formation compared to control at 7 days and at 28 days when it was released at a rate of at least 100% within 28 days and/or when a dose of at least 150 μg, or a dose of at least 2.0 μg/mm², or a dose of at least 610.7 μg/mm of stent length, was released within 28 days from vessel injury (or from implantation).

As shown in Table7, Rivaroxaban released from a stent was more effective at inhibiting fibrin formation compared to control at 7 days and at 28 days when it was released at a rate of at least 88.9% within 7 days and/or when a dose of at least 88.9 μg, or a dose of at least 1.2 μg/mm², or a dose of at least 7.14 μg/mm of stent length, was released within 7 days from vessel injury (or from implantation), and at a rate of at least 92.5% within 28 days and/or when a dose of at least 92.5 μg, or a dose of at least 1.1 μg/mm2, or a dose of at least 6.6 μg/mm of stent length, was released within 28 days from vessel injury (or from implantation).

As shown in Table 10, Rivaroxaban released from a stent was not effective at inhibiting fibrin formation compared to control at 28 days when it was released at a slower rate and/or at a smaller dose.

As shown in Table 10, Rivaroxaban released in combination with an m-TOR inhibitor from a stent was effective at inhibiting fibrin formation compared to control at 7 days and at 28 days only when it was released faster and/or at a larger dose.

As shown in Table 10, Rivaroxaban released in combination with an m-TOR inhibitor from a stent was effective at inhibiting fibrin formation compared to control at 7 days and at 28 days when Rivaroxaban was released at a rate of at least 88.9 μg, or a dose of at least 1.2 μg/mm², or a dose of at least 7.14 μg/mm of stent length, within 7 days, and/or released at a rate of at least 92.5 μg, or a dose of at least 1.1 μg/mm², or a dose of at least 6.6 μg/mm of stent length, within 28 days.

As shown in Table 10, Rivaroxaban released in combination with an m-TOR inhibitor from a stent was effective at inhibiting fibrin formation compared to control at 7 days when Rivaroxaban was released at a rate of at least 68.1 μg, or at rate of 0.84 μg/mm², or at a rate of at least 4.86 μg/mm of stent length, within 7 days.

As shown in Table 10, Rivaroxaban tissue concentration ranges from at least 3.96 ng/mg of tissue adjacent to the stented segment to at least 15 ng/mg of tissue adjacent to the stented segment, within or at 7 days, or within or 28 days from implant (or tissue injury)

It was reported that Rivaroxaban IC₅₀ for factor Xa inhibition to be about 21 nM or 0.0092 ng/mg. As shown in Table 10, the tissue concentration for Rivaroxaban was at least 426 times Rivaroxaban IC 50 for factor Xa inhibition.

TABLE 11
Tissue concentration of Rivaroxaban and Argatroban at 7 days show folds higher (or times
higher) than IC50 for Anti-factor Xa/IIa and antiplatelet for the respective drugs.

| | | | Argatroban* | |
|---|---|---|---|---|
| | Rivaroxaban* | | Tissue | |
| | Tissue | Tissue | concentration | Tissue |
| | concentration at | concentration at | at day 7 in | concentration at |
| Stent coated with combination | day 7 in folds | day 7 in folds | folds higher | day 7 in folds |
| of Rivaroxaban and | higher than IC50 | higher than IC50 | than IC50 of | higher than IC50 |
| Argatroban | of anti-Factor Xa | of anti-platelet | anti-Factor IIa | of anti-platelet |
| 100 μg Argatroban and 100 μg | 5253 | 354 | 835 | 1223 |
| Rivaroxaban in Poly(n-butyl | | | | |
| methacrylate) matrix | | | | |

*Rivaroxaban IC50 for Anti-Factor Xa is 21 nM or 0.00916 ng/mg
*Rivaroxaban IC50 for Tissue factor generated antiplatelet is 312 nM or 0.136 ng/mg
**Argatroban IC50 for Anti-Factor IIa is 21 nM or 0.0107 ng/mg
**Argatroban IC50 for Tissue factor generated antiplatelet is 79 nM or 0.04 ng/mg

Table 11 shows the tissue PK data for Rivaroxaban and Argatroban at or by or within 7 days from implants of stented vessels. It shows Rivaroxaban and Argatroban has therapeutic tissue concentrations in the tissue segment up to 7 days. Table 8 is Rivaroxaban and Argatroban concentration (ng/mg) in the tissue of treated area of the implanted device fold higher than IC₅₀ for anti-Factor Xa or Anti-Factor IIa and anti-platelet. It shows that Rivaroxaban and Argatroban in tissue concentrations have several order of magnitudes, has from 2 to 4 orders of magnitude) of tissue concentration for each of the drugs compared to their IC₅₀, in the treated tissue segments up to 7 days, therefore inhibiting fibrin formation or clot formation on the device surfaces, the stented segment tissue, and the tissue adjacent to the stented segment.

### Example 18: Preparation of Anticoagulant1/Anticoagulant2/mTOR Eluting Stents

Poly(L-lactide acid-co-glycolic acid) polymer was dissolved into dichloromethane at room temperature and vortex until the polymer had uniformly dissolved/dispersed. Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (SS7 arm anticoagulant (Apixaban to Argatroban was 1:1) to poly(L-lactide acid-co-glycolic acid) matrix by weight ratio was 3:1 as a base coat and Siroliums to poly(L-lactide acid-co-glycolic acid) matrix by weight ratio was 2:3 and coated as a top coat), (SS9 arm Siroliums and anticoagulant Apixaban and Argatroban was (1:1:1) to poly(L-lactide acid-co-glycolic acid) by weight ratio was 5:2 on matrix), (SS15 arm Sirolimus and Apixaban and Argatroban were combined in a ratio of (1:1:1) with poly(L-lactide acid-co-glycolic acid) by weight ratio which was (1:2) (by weight of 23 μg Sirolimus. 23 μg Apixaban and 23 μg Argatroban combined with 138 μg poly(L-lactide acid-co-glycolic acid)) and mixed together, and coated as a base coat (drug/polymer matrix as a base coat). In addition. Sirolimus and Apixaban and Argatroban were combined in the ratio of (3:4:4) with poly(L-lactide acid-co-glycolic acid) by weight ratio which was (5:3) and coated as a top layer or coat (drug/polymer matrix top layer or coat). (by weight of 71 μg Sirolimus. 94 μg Apixaban and 94 μg Argatroban and combined with 155 μg poly(L-lactide acid-co-glycolic acid) and coated as a top layer or coat, for cumulative total target drug dose of 117 μg for each anticoagulant and 94 μg for Sirolimus for a 14 mm stent length. (Slider II Arm1 (SS16) Sirolimus and Rivaroxaban and Argatroban were combined together in the ratio of (1:1:1) and were combined with poly(L-lactide acid-co-glycolic acid) by weight ratio which was (1:2) and coated as a base coat (drug/polymer matrix as base coat). In addition. Sirolimus and Rivaroxaban and Argatroban were combined in the ratio of (3:4:4) and combined with poly(L-lactide acid-co-glycolic acid) by weight ratio was (5:3) and coated as a top layer or coat (drug/polymer matrix as top layer or coat). (by weight of 23 μg Sirolimus. 23 μg Rivaroxaban and 23 μg Argatroban and 138 μg poly(L-lactide acid-co-glycolic acid) mixed together and coated as base coat; and by weight of 71 μg Sirolimus. 94 μg Rivaroxaban and 94 μg Argatroban and 155 μg poly(L-lactide acid-co-glycolic acid) mix together in a matrix and coated as top layer or coat, for a total target drug dose of 117 μg for each anticoagulant and 94 μg for Sirolimus for a 14 mm stent length. (Slider II Arm2 (SS17) Sirolimus and Rivaroxaban and Argatroban were combined in a ratio of (4:1:1) and combined with poly(L-lactide acid-co-glycolic acid) by weight ratio which was (1:1) and coated as a base coat (drug/polymer matrix as base coat). In addition. Rivaroxaban and Argatroban were combined in a ratio of (1:1) and combined with poly(L-lactide acid-co-glycolic acid) by weight ratio which was (5:3) and coated as a top layer or coat on the stent (drug/polymer matrix as a top layer or coat). (by weight of 94 μg Sirolimus. 23 μg Rivaroxaban and 23 μg Argatroban and 140 μg poly(L-lactide acid-co-glycolic acid) mixed together and coated as base coat; and by weight of 94 μg Rivaroxaban and 94 μg Argatroban and 113 μg poly(L-lactide acid-co-glycolic acid) were mixed together and coated as top layer or coat, for a total target drug dose of 117 μg for each anticoagulant and 94 μg for Sirolimus for a 14 mm stent length. The preceding doses for SS7, SS9, SS15, SS16, and SS17 were for 14 mm stent lengths. Drug and polymer doses are adjusted accordingly for each stent length. Control was 14 mm stent length eluting 65 μg Novolimus (m-TOR inhibitor). A microprocessor controlled ultrasonic sprayer was used to coat each of the stents' 14 mm length uniformly with each of the drug/polymer matrix solution. After coating, the stents were placed in a 70° C., oven for about 2 hours to remove the solvent. The stents were then mounted on balloon catheters and crimped. The catheters were then inserted in coils and packaged. The pouches were sterilized.

The following Tables 12A-12D describe results from in vivo testing for the following arms of SS7, SS9, SS15, SS16, and SS17.

TABLE 12A
In-vivo cumulative percent drug release profile of Rapamycin,
Apixaban/Rivaroxaban and Argatroban in stented segments.

| | Sample | | | | | | | | |
|---|---|---|---|---|---|---|---|---|---|
| Sample Matrix | Size | Time period | 1 H | 3 H | 24 H | 6 D | 7 D | 28 D | 90 D |
| SS7 Argatroban/Apixaban/ | n = 1 | Apixaban,% | N/A | 30 | 91 | 98 | 99 | 99 | N/A |
| Sirolimus | | Argatroban,% | N/A | 91 | 99 | 98 | 99 | 99 | N/A |
| | | Sirolimus,% | N/A | 50 | 70 | 69 | 73 | 77 | N/A |
| SS9 Argatroban/Apixaban/ | n = 1 | Apixaban,% | N/A | 68 | 97 | 98 | 98 | 99 | N/A |
| Sirolimus | | Argatroban,% | N/A | 79 | 98 | 98 | 98 | 99 | N/A |
| | | Sirolimus,% | N/A | 60 | 91 | 93 | 94 | 97 | N/A |
| SS15 Argatroban/Apixaban/ | n = 5 | Apixaban,% | 49 | 61 | 77 | N/A | 80 | 84(n = 3) | 87(n = 3) |
| Sirolimus | | Argatroban,% | 51 | 63 | 77 | N/A | 80 | 84(n = 3) | 86(n = 3) |
| | | Sirolimus,% | 44 | 55 | 71 | N/A | 81 | 90(n = 3) | 97(n = 3) |
| SS16 Argatroban/Rivaroxaban/ | n = 5 | Rivaroxaban,% | 36 | 47 | 83 | N/A | 86 | 89 | N/A |
| Sirolimus | | Argatroban,% | 35 | 49 | 82 | N/A | 85 | 87 | N/A |
| | | Sirolimus,% | 29 | 44 | 73 | N/A | 87 | 94 | N/A |
| SS17 Argatroban/Rivaroxaban/ | n = 5 | Rivaroxaban,% | 65 | 71 | 86 | N/A | 92 | 94 | N/A |
| Sirolimus | | Argatroban,% | 78 | 80 | 86 | N/A | 91 | 93 | N/A |
| | | Sirolimus,% | 8 | 18 | 63 | N/A | 77 | 87 | N/A |

N/A: Not available

Table 12A: SS7 and SS9 provide a therapeutic composition where about 90% of the factor Xa and factor IIa inhibitors are released from the stent within 24 hours. It also shows that these agents are released substantially completely within about 28 days.

Table 12A: SS15, SS16, and SS17 provide therapeutic compositions where each composition providing a bolus drug release from time of injury and/or implant, and an extended drug release from time of injury and/or implant for each of Apixaban, Rivaroxaban, and Argatroban.

Table 12A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour, within 3 hours, or within 24 hours, from time of injury and/or implant; and the extended drug release extends beyond 7 day, extends beyond 28 days, or extends beyond 90 days from time of injury and/or implant.

Table 12A: SS15 provides a therapeutic composition a bolus drug release phase (or formulation) from time of injury and/or implant and an extended drug release phase (or formulation) from time of injury and/or implant for the combination of Apixaban. Argatroban. and Sirolimus, wherein the bolus drug release occurs within an hour to within 24 hours from time of injury and/or implant and the extended drug release extends beyond 7 day, extends beyond 28 days, or extends beyond 90 days from time of injury and/or implant.

Table 12A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour from time of injury and/or implant and wherein Apixaban bolus release is about 49% within an hour and wherein Argatroban bolus release is about 51% within an hour and the extended drug release of each of the drugs is about 80% within 7 days, about 84% within 28 days, and about 86% within 90 days from time of injury and/or implant. In this arm, the drugs are released or commence release substantially about the same time.

Table 12A: SS16 and SS17 provide therapeutic composition providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and the extended drug release extends beyond 7 day, or extends beyond 28 days from time of injury and/or implant.

Table 12A: SS16 provides a therapeutic composition providing a bolus drug release phase (or formulation) from time of injury and/or implant and an extended drug release phase (formulation) from time of injury and/or implant for the combination of Rivaroxaban.

Argatroban, and Sirolimus, wherein the bolus drug release occurs within an hour to within 24 hours from time of injury and/or implant and the extended drug release extends beyond 7 day, or extends beyond 28 days from time of injury and/or implant. In this arm, the drugs are released or commence release substantially about the same time.

Table 12A: SS16 and SS17 provide therapeutic compositions providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and wherein Rivaroxaban bolus release ranges from 36% to 68% within an hour and wherein Argatroban bolus release ranges from 35% to 78% within an hour and the extended drug release of each of the drugs ranges from 85% to 92% for Rivaroxaban within 7 days, 86%-91% for Argatroban within 7 days, ranges from 89%-94% within 28 days for Rivaroxaban and 87%-93% for Argatroban from time of injury and/or implant to within 28 days.

Table 12A: SS16 and SS17 formulations each has one formulation providing a bolus drug release and another formulation providing an extended drug release for the combination of Rivaroxaban and Argatroban, wherein the bolus drug release occurs within an hour to within 24 hours of injury or implantation and the extended drug release extends beyond 7 day, or extends beyond 28 days.

Table 12A: SS16 and SS17 shows multiple formulations providing a bolus drug release formulation and an extended drug release formulation for the combination of each of Rivaroxaban and Argatroban, wherein the extended release extends beyond 7 day, or extends beyond 28 days.

Table 12A: SS15, SS16, and SS17 Provides therapeutic compositions comprising two drugs/polymer formulations each, wherein each formulation contains at least two drugs: a factor Xa inhibitor and a factor IIA inhibitor. A third drug being an M-tor inhibitor is present in each of the formulations except in SS17 where it is present in only one formulation (base formulation) configured to delay the release of M-tor in SS17 providing a smaller bolus within the first hour for M-tor. All formulations provide an extended release of the drugs beyond 7 days, or beyond 28 days. Arm SS17 factor IIa inhibitor and factor Xa inhibitor commence release prior to the anti-proliferative which was intended/configured to delay commence of its release compared to the other two drugs.

TABLE 12B
Tissue drug concentration (ng/mg) of Apixaban, Rivaroxaban, Argatroban and Rapamycin
in the stented segment tissue at the indicated time points following implantation.

| Sample | Sample | | | | | | | | |
|---|---|---|---|---|---|---|---|---|---|
| Matrix | Size | Time period | 1 H | 3 H | 24 H | 6 D | 7 D | 28 D | 90 D |
| SS7 | n = 1 | Apixaban | N/A | 102.9 | 16.5 | 0.07 | 0.03 | 0.12 | N/A |
| Argatroban/ | | Argatroban | N/A | 54 | 0.75 | 0.09 | 0.05 | 0.15 | N/A |
| Apixaban/ | | Sirolimus | N/A | 7.34 | 4.46 | 1.38 | 0.73 | 1.79 | N/A |
| Sirolimus | | | | | | | | | |
| SS9 | n = 1 | Apixaban | N/A | 91.8 | 4.17 | 0.28 | 2.65 | 1.69 | N/A |
| Argatroban/ | | Argatroban | N/A | 123.1 | 0.18 | 0.33 | 2.54 | 1.76 | N/A |
| Apixaban/ | | Sirolimus | N/A | 40.57 | 0.93 | 1.61 | 3.24 | 2.48 | N/A |
| Sirolimus | | | | | | | | | |
| SS15 | n = 5 | Apixaban | 66.94 ± | 25.31 ± | 13.25 ± | NA | 1.15 ± | 1.28 ± 0.47 | 3.05 ± 1.77 |
| Argatroban/ | | | 27.33 | 11.21 | 10.17 | | 0.52 | (n = 3) | (n = 3) |
| Apixaban/ | | Argatroban | 71.37 ± | 27.65 ± | 15.64 ± | NA | 1.41 ± | 1.69 ± 0.64 | 3.74 ± 1.89 |
| Sirolimus | | | 31.32 | 15.00 | 12.08 | | 0.69 | (n = 3) | (n = 3) |
| | | Sirolimus | 43.22 ± | 23.37 ± | 29.17 ± | NA | 1.54 ± | 1.67 ± 0.22 | 1.28 ± 0.07 |
| | | | 14.73 | 6.88 | 18.65 | | 0.35 | (n = 3) | (n = 3) |
| SS16 | n = 5 | Rivaroxaban | 48.75 ± 25.52 | 21.48 ± 5.80 | 3.67 ± 5.59 | N/A | 0.31 ± 0.24 | 0.34 ± 0.27 | N/A |
| Argatroban/ | | Argatroban | 61.87 ± 24.60 | 32.81 ± 10.96 | 3.80 ± 4.87 | N/A | 0.42 ± 0.32 | 0.52 ± 0.37 | N/A |
| Rivaroxaban/ | | Sirolimus | 45.10 ± 14.77 | 38.30 ± 9.29 | 9.18 ± 5.69 | N/A | 1.46 ± 0.38 | 0.94 ± 0.19 | N/A |
| Sirolimus | | | | | | | | | |
| SS17 | n = 5 | Rivaroxaban | 38.31 ± 16.08 | 26.23 ± 23.50 | 1.31 ± 0.28 | N/A | 1.07 ± 1.88 | 0.52 ± 0.63 | N/A |
| Argatroban/ | | Argatroban | 11.80 ± 2.69 | 8.06 ± 3.48 | 1.19 ± 0.46 | N/A | 1.35 ± 2.41 | 0.67 ± 0.87 | N/A |
| Rivaroxaban/ | | Sirolimus | 21.34 ± 7.51 | 27.32 ± 6.86 | 10.85 ± 3.55 | N/A | 3.80 ± 4.86 | 1.73 ± 1.52 | N/A |
| Sirolimus | | | | | | | | | |

Table 12B shows drug concentration in tissue adjacent to the stented segment for each of the drugs: Apixaban of about 67 ng/mg within one hour, of about 25 ng/mg tissue within 3 hours, of about 1.15 ng/mg tissue within 7 days, 1.28 ng/mg tissue within 28 days, and of about 3 ng/mg tissue within 90 days from time of injury and/or implant; Rivaroxaban of about 38 ng/mg, or of about 49 ng/mg within one hour, of about 21 ng/mg, or of about 26 ng/mg tissue within 3 hours, of about 0.3 ng/mg, or of about 1.1 ng/mg tissue within 7 days, of about 0.34 ng/mg, or of about 0.52 ng/mg tissue within 28 days, from time of injury and/or implant: Argatroban of about 12 ng/mg, of about 62 ng/mg tissue, or of about 71 ng/mg tissue within 1 hour, of about 8 ng/mg tissue, of about 33 ng/mg tissue, or of about 27 ng/mg tissue within 3 hours, of about 0.42 ng/mg tissue, of about 1.35 ng/mg tissue, or of about 1.41 ng/mg tissue within 7 days, of about 0.52 ng/mg tissue, of about 0.67 ng/mg tissue, or of about 1.69 ng/mg tissue within 28 days, and of about 3.74 ng/mg tissue within 90 days from time of injury and/or implant; and Sirolimus of about 21 ng/mg tissue, of about 45 ng/mg tissue, or of about 43 ng/mg tissue within one hour, of about 27 ng/mg tissue, or about 38 ng/mg tissue, or of about 24 ng/mg tissue within 3 hours, of about 1.46 ng/mg tissue, of about 3.8 ng/mg tissue, or of about 1.54 ng/mg tissue within 7 days, of about 0.94 ng/mg tissue, of about 1.73 ng/mg tissue, or of about 1.67 ng/mg tissue within 28 days, and of about 1.28 ng/mg tissue within 90 days, from time of tissue injury and/or implant.

TABLE 12C
In Vivo drug remaining on stent (μg) and average cumulative percentage releases (%) of Apixaban,
Rivaroxaban, Argatroban and Sirolimus in the stent at the indicated time points following implantation.

| Sample | SS15 Argatroban/Apixaban/ | | | SS16 Argatroban/Rivaroxaban/ | | | SS17 Argatroban/Rivaroxaban/ | | |
|---|---|---|---|---|---|---|---|---|---|
| matrix* | Sirolimus in base coat and | | | Sirolimus in base coat and | | | Sirolimus in base coat and | | |
| (n = 5) | in topcoat | | | in topcoat | | | in topcoat | | |
| Drug | Apixaban | Argatroban | Sirolimus | Rivaroxaban | Argatroban | Sirolimus | Rivaroxaban | Argatroban | Sirolimus |
| remaining | | | | | | | | | |
| on stent, μg | | | | | | | | | |
| & | | | | | | | | | |
| percentage | | | | | | | | | |
| released | | | | | | | | | |
| (%) | | | | | | | | | |
| Time | | | | | | | | | |
| (hrs) | | | | | | | | | |
| 0 | 119 | 123 | 96 | 120 | 119 | 90 | 121 | 121 | 96 |
| 1 H | 61 ± 4.8 | 60 ± 5.5 | 54 ± 2.8 | 76 ± 7.1 | 77 ± 59.2 | 63 ± 4.7 | 43 ± 3.5 | 26 ± 0.8 | 88 ± 1.5 |
| | (49%) | (51%) | (44%) | (36%) | (35%) | (29%) | (65%) | (78%) | (8%) |
| 3 H | 46 ± 5.2 | 46 ± 5.1 | 43 ± 3.9 | 64 ± 9.5 | 61 ± 13.9 | 50 ± 6.1 | 34 ± 5.1 | 24 ± 1.9 | 79 ± 3.5 |
| | (61%) | (63%) | (55%) | (47%) | (49%) | (44%) | (71%) | (80%) | (18%) |
| 24 H | 27 ± 3.6 | 28 ± 1.4 | 28 ± 0.8 | 21 ± 4.8 | 21 ± 1.6 | 24 ± 7.1 | 17 ± 5.9 | 17 ± 3.8 | 36 ± 7.3 |
| | (77%) | (77%) | (71%) | (83%) | (82%) | (73%) | (86%) | (86%) | (63%) |
| 7 D | 24 ± 1.4 | 25 ± 1.0 | 18 ± 0.3 | 16 ± 2.5 | 18 ± 1.7 | 12 ± 1.2 | 9 ± 0.3 | 11 ± 0.4 | 22 ± 0.5 |
| | (80%) | (80%) | (81%) | (86%) | (85%) | (87%) | (92%) | (91%) | (77%) |
| 28 D | 19 ± 0.9 | 20 ± 6.1 | 9 ± 0.3 | 14 ± 1.0 | 15 ± 0.8 | 5 ± 0.4 | 7 ± 0.4 | 9 ± 0.7 | 13 ± 0.7 |
| | (84%) | (84%) | (90%) | (89%) | (87%) | (94%) | (94%) | (93%) | (87%) |
| 90 D | 16 ± 1.6 | 17 ± 0.6 | 3 ± 0.1 | N/A | N/A | N/A | N/A | N/A | N/A |
| | (87%) | (86%) | (97%) | | | | | | |

N/A: Not available
*SS15 28 D and 90 D (n = 3)

TABLE 12D
In Vivo drug concentration (ng/mg) in the tissue within 5 mm proximal and 5
mm distal (tissue adjacent to the stented segment) to the stented segment.

| Sample | | | | | | | | | |
|---|---|---|---|---|---|---|---|---|---|
| Matrix | | | | | | | | | |
| (Sample | | 1 Hour | | 3 Hour | | 1 Day | | 6 Day | |
| Size) | Drug | Proximal | Distal | Proximal | Distal | Proximal | Distal | Proximal | Distal |
| SS15 | Apixaban, | 7.86 ± | 3.53 ± | 5.15 ± | 6.77 ± | 0.14 ± | 0.36 ± | N/A | N/A |
| (n = 5 | ng/mg | 3.01 | 1.95 | 1.59 | 2.22 | 0.08 | 0.29 | | |
| except | Aratroban | 8.57 ± | 3.84 ± | 5.43 ± | 6.46 ± | 0.05 ± | 0.17 ± | N/A | N/A |
| 28 D & | ng/mg | 3.66 | 2.42 | 2.67 | 3.14 | 0.03 | 0.09 | | |
| 90 D | Sirolimus | 2.82 ± | 1.66 ± | 3.52 ± | 3.81 ± | 0.12 ± | 3.18 ± | N/A | N/A |
| n = 3) | ng/mg | 1.15 | 0.78 | 0.51 | 0.61 | 0.11 | 3.01 | | |
| SS16 | Rivaroxaban, | 2.63 ± | 6.51 ± | 1.72 ± | 2.16 ± | 0.09 ± | 0.09 ± | N/A | N/A |
| (Slider II | ng/mg | 1.14 | 2.45 | 0.53 | 0.83 | 0.03 | 0.02 | | |
| Arm 1 | Argatroban, | 3.74 ± | 7.87 ± | 2.48 ± | 2.63 ± | 0.07 ± | 0.09 ± | N/A | N/A |
| (n = 5)) | ng/mg | 1.93 | 3.04 | 0.36 | 0.87 | 0.03 | 0.03 | | |
| except | Sirolimus, | 2.30 ± | 5.25 ± | 2.62 ± | 3.78 ± | 0.18 ± | 1.10 ± | N/A | N/A |
| 3 H | ng/mg | 1.22 | 1.62 | 0.82 | 0.81 | 0.06 | 0.44 | | |
| n = 4, | | | | | | | | | |
| 28 d | | | | | | | | | |
| n = 6) | | | | | | | | | |
| SS17 | Rivaroxaban, | 1.52 ± | 2.61 ± | 3.00 ± | 2.72 ± | 0.09 ± | 0.07 ± | N/A | N/A |
| (Slider II | ng/mg | 0.70 | 1.38 | 1.50 | 1.66 | 0.05 | 0.02 | | |
| Arm2 | Argatroban | 0.65 ± | 0.91 ± | 0.68 ± | 0.87 ± | 0.04 ± | 0.03 ± | N/A | N/A |
| (n = 5 | | 0.23 | 0.51 | 0.28 | 0.52 | 0.01 | 0.01 | | |
| except | Sirolimus, | 1.39 ± | 1.81 ± | 2.49 ± | 3.95 ± | 0.26 ± | 0.86 ± | N/A | N/A |
| 3 H | ng/mg | 0.83 | 0.83 | 1.02 | 3.09 | 0.27 | 0.48 | | |
| n = 4)), | | | | | | | | | |
| 28 d | | | | | | | | | |
| n = 6) | | | | | | | | | |

| Sample | | | | | | | |
|---|---|---|---|---|---|---|---|
| Matrix | | | | | | | |
| (Sample | | 7 Day | | 28 Day | | 90 Day | |
| Size) | Drug | Proximal | Distal | Proximal | Distal | Proximal | Distal |
| SS15 | Apixaban, | 0.01 ± | 0.01 ± | 0.04 | 0.02 | 0.0003 ± | 0.0002 ± |
| (n = 5 | ng/mg | 0.01 | 0.01 | (n = 1) | (n = 1) | 0.0002 | 0.0001 |
| except | | | | | | (n = 2) | (n = 3) |
| 28 D & | Aratroban | | | BQL | BQL | BQL | |
| 90 D | ng/mg | | | (n = 2) | (n = 2) | (n = 1) | |
| n = 3) | Sirolimus | 0.01 ± | 0.01 ± | 0.04 | 0.02 | 0.008 | 0.0006 ± |
| | ng/mg | 0.01 | 0.003 | (n = 1) | (n = 1) | (n = 1) | 0.0004 |
| | | | | | | BQL | (n = 2) |
| | | | | | | (n = 2) | |
| SS16 | Rivaroxaban, | BQL | BQL | 0.02 | 0.02 | N/A | N/A |
| (Slider II | ng/mg | | | (n = 1); | (n = 1); | | |
| Arm 1 | | | | BQL | BQL | | |
| (n = 5)) | | | | (n = 5) | (n = 5) | | |
| except | Argatroban, | BQL | BQL | BQL | BQL | N/A | N/A |
| 3 H | ng/mg | | | | | | |
| n = 4, | Sirolimus, | 0.04 ± | 0.55 ± | 0.05 ± | 0.11 ± | N/A | N/A |
| 28 d | ng/mg | 0.03 | 0.22 | 0.05 | 0.05 | | |
| n = 6) | | | | (n = 4); | | | |
| | | | | BQL | | | |
| | | | | (n = 2) | | | |
| SS17 | Rivaroxaban, | 0.01 | 0.01 | 0.02 ± | BQL | N/A | N/A |
| (Slider II | ng/mg | (n = 1); | (n = 1): | 0.001 | | | |
| Arm2 | | BQL | BQL | (n = 2); | | | |
| (n = 5 | | (n = 4) | (n = 4) | BQL | | | |
| except | | | | (n = 4) | | | |
| 3 H | Argatroban | BQL | BQL | BQL | BQL | N/A | N/A |
| n = 4)), | | | | | | | |
| 28 d | Sirolimus, | 0.01 | 0.34 ± | 0.03 ± | 0.13 ± | N/A | N/A |
| n = 6) | ng/mg | (n = 3); | 0.21 | 0.02 | 0.06 | | |
| | | BQL | | (n = 4); | | | |
| | | (n = 2) | | BQL | | | |
| | | | | (n = 2) | | | |

BQL: Below Quantification Limit
N/A: Not Available

### Example 19: In Vivo Animal Study of Anticoagulant1/Anticoagulant2/mTOR Eluting Stents

The test drug eluting stent systems containing anticoagulants were prepared as described in Example 4 and were evaluated at 28 days and 90 days following implantation in a porcine coronary artery. The control device was the Novolimus (m-TOR) eluting DESyne X2 stent.

The porcine artery was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

All animals were pretreated with aspirin (325 mg) and Clopidogrel (75 mg) per oral dose beginning at least 3 days prior to the intervention and continuing for the duration of the study. After induction of anesthesia, the left or right femoral artery was accessed using standard techniques and an arterial sheath was introduced and advanced into the artery. Vessel angiography was performed under fluoroscopic guidance, a 7 Fr, guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately sized stent (3.0×14 mm or 3.5×14 mm) was advanced to the deployment site. The balloon was inflated at a steady rate to a pressure sufficient to achieve a balloon to artery ratio of approximately 1.1 to 1.0 but less than 1:2:1. Pressure was maintained for approximately 10 seconds before the balloon was deflated. Each pig was implanted with 3 test devices and one control device in the coronary arteries. Each time point a whole blood was drawn from animals for blood drug concentration test.

Follow up angiography imaging was performed at the designated endpoint for each of the animals. Quantitative coronary angiographic analysis was performed and the average percent diameter stenosis values and late lumen loss for the test arms and control DESyne X2 for the 28 days and 3-month time points are shown in Table 13.

Upon completion of follow-up angiography imaging, the animals were euthanized. The hearts were harvested from each animal. Any myocardial lesions or unusual observations were reported. The coronary arteries were perfused with 10% buffered formalin at 100 to 120 mm Hg with the animal's ear tag until processed for histology.

Stented portions of coronary arteries were embedded in methyl methacrylate (MMA), then divided into a target of three blocks of approximately similar length (about 4 mm), identified as proximal, mid and distal segments. From three blocks, 3 to 5 cuts were made for histology evaluation.

Quantitative histopathological evaluation of stented artery sections was then performed and scored as indicated. The mean of each section was recorded and then averaged to provide a mean score per stent for the different parameters (Table 14). The smaller the score, the better the efficacy.

### Fibrin (Strut-by-Strut)

0=absent, or rare minimal spotting around struts
1=fibrin in small amounts, localized only around struts
2=fibrin moderately abundant or denser, extending beyond struts
3=abundant, dense fibrin, bridging between struts

Each strut in the section was scored: the mean fibrin score for each section was calculated and reported. The mean of the section means was calculated and reported, providing a mean fibrin score per stent.

Injury Based on Schwartz et al. J am Coll Cardiol 1992; 19:267-274. (Strut-by-Strut):
0=IEL intact
1=IEL lacerated
2=media completely lacerated
3=EEL lacerated

Each strut in the section as scored and the mean injury score for each section was calculated and reported. The mean of the section means was calculated and reported, providing a mean injury score per stent.

### Inflammation (Strut-by-Strut)

0=no or very few (<3) inflammatory cells around strut
1=few (˜4-10) inflammatory cells around strut
2=many (>10) inflammatory cells around strut, can extend into but does not efface surrounding tissue
3=many (>10) inflammatory cells, effacing surrounding tissue

Each strut in the section was scored and the mean inflammation score for each section was calculated and reported. The mean of the section means was calculated and reported, providing a mean inflammation score per device.

TABLE 13
Histopathology Scores and Quantitative Coronary Angiography data Apixaban/Rivaroxaban,
Argatroban and Sirolimus releasing 14 mm stents at 7 days, 28 days and 3 months

| | | | | | | Diameter | |
|---|---|---|---|---|---|---|---|
| Time Point | | Device | Injury | Inflammation | Fibrin | Stenosis % | LLL, mm |
| 7 | Day | SS7(n = 1) | 0.15 | 1.52 | 0.62 | N/A | N/A |
| | | SS9(n = 1) | 0.61 | 1.66 | 0.44 | N/A | N/A |
| 28 | Day | SS7(n = 1) | 0.21 | 0.49 | 0.94 | 16.7 | 0.71 |
| | | SS9(n = 1) | 0.36 | 0.32 | 0.64 | 21.3 | 0.76 |
| | | SS15 (n = 3) | 0.38 ± 0.34 | 0.62 ± 0.12 | 1.67 ± 0.38 | 19.8 ± 4.1 | 0.45 ± 0.15 |
| | | DESyne | 1.34 | 1.83 | 1.87 | 51.6 | 1.14 |
| | | X2(n = 1) | | | | | |
| | | SS16(Slider II | 0.91 ± 0.95 | 1.52 == 1.03 | 1.90 ± 0.26 | 19.4 ± 13.8 | 0.72 ± 0.32 |
| | | Arm1, n = 6) | | | | | |
| | | DESyne | 1.17 ± 1.60 | 1.97 ± 1.46 | 1.32 ± 1.11 | 36.6 ± 27.3 | 0.99 ± 0.48 |
| | | X2(n = 2) | | | | | |
| | | SS17(Slider II | 1.01 ± 0.47 | 1.46 ± 0.43 | 1.88 ± 0.48 | 33.1 ± 12.8 | 0.86 ± 0.34 |
| | | Arm2, n = 6) | | | | | |
| | | DESyne X2 | 0.87 ± 0.74 | 1.59 ± 0.95 | 1.93 ± 0.31 | 21.9 ± 11.6 | 0.71 ± 0.62 |
| | | (n = 2) | | | | | |
| 3 | Month | SS15 (n = 3) | 0.15 ± 0.18 | 0.52 ± 0.19 | 0.08 ± 0.06 | 18.2 ± 9.3 | 0.25 ± 0.29 |
| | | DESyne | 0.43 | 0.59 | 0.76 | 22.3 | 0.52 |
| | | X2(n = 1) | | | | | |

LLL is an indicator of the amount cell proliferation or inhibition potency. It is used to measure efficacy between drugs for proliferation inhibition in mammalian arteries. The smaller the LLL, the better the efficacy of the drug.

As shown in Table 13, SS15 composition providing the combination of Sirolimus, Apixaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days, and at 90 days. This was an unexpected finding for the test SS15 stents in comparison to the control DESyne X2 stents at the 28-day time point and/or at 90 days.

In an unexpected finding, SS15 stents composition eluting Apixaban, Argatroban, and the M-Tor inhibitor rapamycin exhibited more efficacy at inhibiting one or more of the following at 28 days and/or 90 day time points: cell proliferation, inflammation, injury, fibrin formation inhibition, and fibrin dissolution acceleration.

The LLL is an indicator of the amount cell proliferation or inhibition potency. It is used to measure efficacy between drugs for proliferation inhibition in mammalian arteries. The smaller the LLL, the better the efficacy of the drug.

As shown in Table 14, SS16 shows the combination of Sirolimus, Rivaroxaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days.

As shown in Table 13, SS17 composition configured to delay the release and tissue concentration of rapamycin within the first 1 hour and/or within the first 3 hours by incorporating rapamycin in the base coating shows the combination of Sirolimus, and/or lower tissue concentration of Rivaroxaban and Argatroban within at least the first hour showed less inhibition of SMC proliferation at 28 days.

### Example 20: Ex Vivo Testing of Drug Eluting Stent Compared with 2 Anticoagulants and mTOR Eluting Stents

The thrombogenicity of a drug eluting stent system with two anticoagulant Apixaban and Argatroban in combination with rapamycin at two different loading drug doses was evaluated at 1 hour in an arteriovenous ex vivo shunt in a porcine model wherein the devices were deployed in a blood compatible polymeric tubing.

The control stents were 16-o-demethyl rapamycin m-TOR inhibitor (Novolimus) drug eluting coronary stent (DESyne, Elixir) and m-TOR inhibitor Zotarolimus eluting coronary stent (Resolute, Medtronic, USA).

The test arm for this experiment were SS9, SS9*, and SS10* and were manufactured as follows: Each polymer solution and each drug solutions were combined together ((Sirolimus and anticoagulant Apixaban and Argatroban was 1:1:1) to poly(L-lactide acid-co-glycolic acid) by weight ratio was 5:2 matrix) according to the target drug dose of 235 μg for each anticoagulant and 94 μg for Sirolimus for SS9, SS9* test arm was about ⅓ of each of the drugs dose as follows: Sirolimus and anticoagulant Apixaban and Argatroban was 1:1:1) to poly(L-lactide acid-co-glycolic acid) by weight ratio was 5:2 on matrix) according to the target drug dose of 78.3 μg for each anticoagulant and 31.3 μg for Sirolimus, and SS10* arm was Sirolimus and anticoagulant Apixaban and Argatroban was 1:1:3) to poly(L-lactide acid-co-glycolic acid) by weight ratio was 5:2 matrix) according to the target drug dose of 39.2 μg for Apixaban and 117.5 μg for Argatroban and 31.3 μg for Sirolimus.

A microprocessor controlled ultrasonic sprayer was used to coat each of the stents 14 mm length uniformly with each of the drug/polymer matrix solution. After coating, the stents were placed in a 70° C., oven for about 2 hours to remove the solvent. The stents were then mounted on balloon catheters and crimped. The catheters were then inserted in coils and packaged. The pouches were sterilized.

The ex-vivo shunt model to evaluate thrombogenicity has been extensively employed to evaluate the biocompatibility of different drug eluting stents (Waksman et al. Circ Cardiovasc Interv. 2017:10:e004762, Otsuka et al. J Am Coll Cardiol Inty 2015:8:1248-60, Lipinski et al. EuroInterv 2018; Jaa-369 2018) The animals were housed and cared for in accordance with the Guide for the Care and Use of Laboratory Animals as established by the National Research Council.

The two pigs that were employed in this study did not receive any aspirin or clopidogel pretreatment. Further all procedures were performed in the absence of any anticoagulant including heparin. After induction of anesthesia, an arterial bypass shunt from the femoral artery to the femoral vein was created. Blood flow was established through the shunt. Flow rates through the shunt was continuously monitored during the procedure with a flow probe that was placed on the shunt tubing proximal to the arterial flow.

In the first pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 3 shunts with only one stent SS9* or SS9 in each shunt was tested with a perfusion time of 1 hour for each of the shunts.

In the second pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 2 shunts with only one stent SS10* in each shunt was tested with a perfusion time of 1 hour for each of the shunts.

Promptly following perfusion in each of the shunts, the tubing containing the stents was gently rinsed with saline under gravity flow and then fixed in situ with 10% buffered formalin in order to anchor the thrombus, if any, that are deposited on the stent surface.

The stents were then removed from the tubing and bisected longitudinally. Low magnification photographs of the luminal side of two halves of the control and test stents were recorded.

The two halves of the stents were then processed for scanning electron microscopy (SEM) so as to examine the thrombus on the luminal side of the stent. Low (15×) and high (200×) magnification images of the stent surface were captured to evaluate the extent of thrombus deposition on the luminal surface of the stent.

Significant number of thrombus was observed on the luminal surface (inner surface) of the control DES stents as seen on the low and high magnification SEM images whereas there was little or no thrombus deposits on the test stents with combinations of Apixaban and Argatroban and m-TOR. The number of thrombus deposits on the control and test stents as evaluated from SEM images are shown in Table 14. The data shows that the combinations of Apixaban and Argatroban and m-TOR inhibited thrombus formation in the shunt model better than control.

Table 14 shows several therapeutic compositions of factor Xa inhibitor, factor IIa, and M-tor inhibitor releasing stents had less thrombus (clot formation) compared to M-Tor inhibitor alone releasing stents.

The composition comprising a combination of factor Xa inhibitor, a factor II inhibitor and an anti-proliferative were surprisingly more effective than the anti-proliferative alone.

TABLE 14
Thrombus deposits on the control and test
stents as evaluated from SEM images

| | | | Number of |
|---|---|---|---|
| | | | Thrombus |
| Animal # | Control/Test DES | Device | deposits |
| 1 | Control | DESyne-1 | 18 |
| | | DESyne-2 | 40 |
| | | Resolute | 14 |
| | Test - Apixaban:Argatroban(1:1) | SS9* | 4 |
| | | SS9* | 0 |
| | | SS9 | 3 |
| 2 | Control | DESyne-1 | 17 |
| | | DESyne-2 | 17 |
| | | Resolute | 15 |
| | Test - Apixaban:Argatroban(1:3) | SS10* | 2 |
| | | SS10* | 2 |

### Example 21: Preparation of Amino Acid and Anticoagulant of Apixaban or Rivaroxaban and Argatroban and a Spacer Eluting Stents

A surface-binding moiety, and one or more cell adhesion amino acid via one or more linkers could make the amino acid more hydrophobic, which can extend anticoagulant Apixaban or Rivaroxaban and Argatroban release in a biocompatible environmental and inhibit inflammation effectively. For example, dihydroxyphenylalanine and L-lysine is immobilized onto 316L stainless steel with polyethylene glycol molecule as spacer arm by using cold plasma-induced grafting technique. To immobilize peptide more effectively, polyethylene glycol is first coated onto 316LSS. After L-lysine immobilized on the stent surface, anticoagulant Apixaban or Rivaroxaban and Argatroban can be coated on the stent. After coating, the stents are placed in a vacuum oven for about 12 hours to remove the solvent. The stents are then mounted on balloon catheters and crimped. The catheters are then inserted in coils and packaged. The pouches are sterilized.

### Example 22: Preparation of Crosslinked Peptide and Anticoagulant Apixaban or Rivaroxaban and Argatroban Eluting Stents

Efficient stent implantation depends in part on avoiding the aggregation of platelets in the blood vessels and appropriate proliferation of endothelial cells and controlled proliferation of smooth muscle cells, which reduces the development of pathology, such as neointimal hyperplasia, thrombosis, and restenosis. Peptide immobilized on the stent surface can make stent more biocompatible. A crosslinked peptide will have a folded structure wrapped the anticoagulant Apixaban or Rivaroxaban and Argatroban inside: once it is inside body wet environmental, it will unfold and release the Anticoagulant Apixaban or Rivaroxaban and Argatroban.

The crosslinking of peptide is achieved with oxidizing agents, such as periodate ion and Fe(III) on the stent surface, resulting in oxidative crosslinking (covalent crosslinking) and coordinative one (non-covalent crosslinking), respectively with dopamine, producing adhesive peptide on the stent surface.

After coating, the stents are placed in a vacuum oven for about 12 hours to remove the solvent. The anticoagulant can then be coated on the stent. After coating, the stentsare placed in a vacuum oven for about 12 hours to remove the solvent. The stents are then mounted on balloon catheters and crimped. The catheters are then inserted in coils and packaged. The pouches are sterilized.

### Example 23: Preparation of Polypeptide Anticoagulant Apixaban or Rivaroxaban and Argatroban Eluting Stents

Nature provides us with unusual adhesives in the form of L-DOPA-containing proteins, existing within the mussel feet and commonly called “mussel foot proteins.” A unique property of these bioadhesives is their ability to adhere to many different surfaces, even in a wet environment. The modification of stents and other implantable device surfaces with such adhesive polypeptides can provide for rapid endothelialization after implantation. The surface-binding cell adhesion polypeptides are deposited on the surface, forming an amino-containing hydrophobic coating to enhance the corrosion resistance by a surface binding moiety, such as 3,4-dihydroxyphenylalanine (DOPA). Catechol moieties (3,4-dihydroxyphenyl) in DOPA are attached to cell adhesion polypeptides. Attachment may be directly to a cell adhesion polypeptide or indirect, for example via a linker. Alternatively, this modified polypeptide can be directly coated on the stent surface with anticoagulant Apixaban or Rivaroxaban and Argatroban. After coating, the stents are placed in a vacuum oven for about 12 hours to remove the solvent. The stents are then mounted on balloon catheters and crimped. The catheters are then inserted in coils and packaged. The pouches are sterilized.

### Example 24: Preparation of Polysaturated Fatty Acid Poly(Glycerol Sebacic Acid) and Anticoagulant Apixaban or Rivaroxaban and Argatroban Eluting Stents

Poly(glycerol sebacic acid) is a simple glycerol-ester polysaturated fatty acid created from the basic mammalian metabolites of glycerol and sebacic acid. Poly(glycerol sebacic acid) coating in the stent has enhanced mechanical properties, improved biocompatibility, and antimicrobial properties. Poly(glycerol sebacic acid) is easily reducible in a wide range of solvents (e.g., ethyl acetate, THF, acetone, 1,3-dioxolane, and various alcohols) resulting in a solution that can be used in dip and spray coating applications.

Poly(glycerol sebacic acid) is dissolved into tetrahydrofuran and dichloromethane (THF:DCM) at room temperature or heated when needed, vortexed until the polymer had uniformly dissolved/dispersed. Anticoagulant (Apixaban/or Rivaroxaban & Argatroban) are placed in a vial and dissolved in dichloromethane/Methanol at room temperature and vortex until all the drug is uniformly dissolved/dispersed. Each polymer solution and each drug solutions are combined (anticoagulant (Apixaban/Rivaroxaban & Argatroban with weight ratio 1 to 1) to Poly(glycerol sebacic acid) by weight ratio is 3:1) according to the target drug dose.

The stent can optionally undergo surface treatment if the surface is not porous (i.e, plasma treatment or other surface friction treatment).

A microprocessor-controlled ultrasonic sprayer is used to coat each of the stents' 14 mm length uniformly with each of the drug/polymer matrix solutions. After coating, the stents are placed in a vacuum chamber to remove the solvent. The stents are then mounted on balloon catheters and crimped. The catheters are then inserted in coils and packaged. The pouches are sterilized.

### Example 25: Preparation of Crosslinked Polyunsaturated Fatty Acid and Anticoagulant Apixaban or Rivaroxaban, Argatroban Eluting Stents

Polyunsaturated fatty acids (PUFAs) such as Omega-3 or Omega-6 can modify platelet responsiveness to dual antiplatelet therapy in stable coronary artery disease patients undergoing percutaneous coronary intervention. Higher platelet inhibition might be achieved with increased doses of polyunsaturated fatty acids coated on the stent surface.

Polyunsaturated fatty acid (PUFA) is precure under Geotrichum sp. Lipase at 40° C., for 8-10 hours or stir at 90° C., and oxygen without enzyme for 24 hours. This partially crosslinked PUFA is washed with water and used for stent coating. This jelly PUFA product and anticoagulant Apixaban or Rivaroxaban, Argatroban are dissolved in a mixture of dichloromethane and methanol. Each partially crosslinked PUFA jelly solution and each drug solutions are combined (anticoagulant (Apixaban & Argatroban with weight ratio 1 to 1) to solid PUFA by weight ratio is 3:1) according to the target drug dose.

The stent can optionally undergo surface treatment if the surface is not porous (i.e, plasma treatment or other surface friction treatment).

A microprocessor-controlled ultrasonic sprayer is used to coat each of the stents' 14 mm length uniformly with each of the drug/polymer matrix solutions. After coating, the stents are cured at 90° C., for 24 hours in a convection oven. The stents are then mounted on balloon catheters and crimped. The catheters are then inserted in coils and packaged. The pouches are sterilized.

### Example 26: Preparation of Anticoagulant1/Anticoagulant2/mTOR Cationic Anti-Coagulation Enhancer Polyethylenimine Eluting Stents

Poly(L-lactide acid-co-glycolic acid) polymer was dissolved into dichloromethane at room temperature and vortex until the polymer had uniformly dissolved/dispersed. Linear polyethylenimine polymer was dissolved into methanol at room temperature and vortex until the polymer had uniformly dissolved/dispersed and diluted with dichloromethane. Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (Sirolimus and Rivaroxaban and Argatroban were combined together in the ratio of (1:1:1) and were combined with poly(L-lactide acid-co-glycolic acid) by weight ratio which was (1:2) and coated as a base coat (drug/polymer matrix as base coat). In addition. Sirolimus and Rivaroxaban and Argatroban were combined in the ratio of (3:4:4) and combined with poly(L-lactide acid-co-glycolic acid) by weight ratio which was (5:3) and coated as a middle layer or coat (drug/polymer matrix as middle layer or coat). (by weight of 23 μg Sirolimus. 23 μg Rivaroxaban and 23 μg Argatroban and 138 μg poly(L-lactide acid-co-glycolic acid) mixed together and coated as base coat; and by weight of 71 μg Sirolimus. 94 μg Rivaroxaban and 94 μg Argatroban and 155 μg poly(L-lactide acid-co-glycolic acid) mix together in a matrix and coated as middle layer or coat, for a total target drug dose of 117 μg for each anticoagulant and 94 μg for Sirolimus for a 14 mm stent length. Lastly, linear polyethylenimine polymer combined with poly(L-lactide acid-co-glycolic acid) by weight ratio which was (1/1 to 2/1 based on the coating integrity and drug elution profile) and coated as a top layer or coat.

A microprocessor controlled ultrasonic sprayer was used to coat each of the stents' 14 mm length uniformly with each of the drug/polymer matrix solution. If needed, after coating, the stents were placed in a 70° C., oven for about half hours to remove the solvent after base coat and middle coat. After top coating, the stents were placed in a 70° C., oven for about 2 hours to remove the solvent. The stents were then mounted on balloon catheters and crimped. The catheters were then inserted in coils and packaged. The pouches were sterilized.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A therapeutic composition comprising at least one chelating agent in combination with at least one of the following additional substances:
(a) a cationic anti-coagulation enhancer;
(b) an anti-coagulant;
(c) an mTOR inhibitor;
(d) paclitaxel or a salt, isomer, solvate, derivative, analog, metabolite, or prodrug thereof; or
(e) an antiplatelet drug.

3. The therapeutic composition of claim 2, wherein the at least one chelating agent in the therapeutic composition is formulated to deplete calcium.

4. The therapeutic composition of claim 3, wherein the at least one chelating agent is selected from the group consisting of: ethylenediaminetetraacetic acid (EDTA), calcium disodium edetate, magnesium dipotassium edetate, magnesium di sodium edetate, di sodium edetate, tetrasodium edetate, trisodium edetate, monoammonium EDTA salt, diammonium EDTA salt, triammonium EDTA salt, benzyldimethyltetradecylammonium EDTA salt, tridodecylmethylammonium EDTA salt, other benzalkonium EDTA salt, tetra acetoxymethyl ester EDTA, ethyleneglycoltetraacetic acid (EGTA), 2,3-dimercaptopropanesulfonic acid (DMPS), thiamine tetrahydrofurfuryl disulfide (TTFD), dimercaptosuccinic acid (DMSA), diethylenetriaminepentaacetate (DTP A), hydroxy ethylethylenediaminetriacetic acid (HEEDTA), diaminocyclohexanetetraacetic acid (CDTA), 1,2-bis(2-aminophenoxy) ethane-N,N,N′,N′-tetraacetic acid (BAPTA), deferoxamine (DFO), a surfactant-EDTA complex, quaternary ammonium EDTA salt, benzalkonium EDTA salt, EDTA complex, and salts, analogues, solvates, hydrates or derivatives of any of the preceding elements.

5. The therapeutic composition of claim 4, wherein the at least one chelating agent consists essentially of ethylenediaminetetraacetic acid (EDTA).

6. The therapeutic composition of claim 2, wherein the cationic anticoagulation enhancer is selected from the group consisting of: magnesium stearate and other magnesium salts, monoammonium salt, diammonium salt, triammonium salt, benzyldimethyltetradecylammonium salt, tridodecylmethylammonium salt, a benzalkonium, and analogues, solvates, hydrates, or derivatives of any of the preceding elements.

7. The therapeutic composition of claim 2, wherein the cationic anticoagulation enhancer is selected from the group consisting of: a cationic polymer, poly(L-lysine) (PLL), linear polyethyleneimine (PEI), branch polyethyleneimine (PEI), chitosan, PAMAM dendrimers, poly(2-dimethylamino)ethyl methacrylate (pDMAEMA), protamine, polylysine, a polybetaaminoester (PBAE), Histone, ethylenediamine, methylenediamine, ammonium chloride, melamine, histamine, histidine, and analogues, solvates, hydrates and derivatives of any of the preceding.

8. The therapeutic composition of claim 2, wherein the cationic anticoagulation enhancer consists essentially of benzyldimethyltetradecylammonium chloride.

9. The therapeutic composition of claim 2, wherein the cationic anticoagulation enhancer consists essentially of linear polyethyleneimine (PEI).

10. The therapeutic composition of claim 2, wherein the therapeutic composition further comprises a factor XI/XIa inhibitor or a protein Z-dependent protease inhibitor consisting essentially of milvexian.

11. The therapeutic composition of claim 2, wherein the therapeutic composition is formulated to release the anti-coagulant at a rate equal to that of the at least one chelating agent.

12. The therapeutic composition of claim 2, wherein the therapeutic composition is formulated to release the anti-coagulant at a rate slower than that of the at least one chelating agent.

13. The therapeutic composition of claim 2, wherein the therapeutic composition is formulated to release at least one anti-coagulant at a rate faster than that of the at least one chelating agent.

14. The therapeutic composition of claim 2, wherein the anticoagulant is selected from the group consisting of a direct factor IIa inhibitor and a direct factor Xa inhibitor.

15. The therapeutic composition of claim 14, wherein the direct factor IIa inhibitor is selected from the group consisting of argatroban, dabigatran, ximelagatran, melagatran, efegatran, inogatran, atecegatran metoxil (AZD-0837), hirudin, hirudin analogs, bivalirudin, desirudin, and lepirudin.

16. The therapeutic composition of claim 15, wherein the direct factor IIa inhibitor comprises argatroban or a salt, isomer, solvate, analog, derivative, metabolite, or prodrug thereof.

17. The therapeutic composition of claim 14, wherein the direct factor Xa inhibitor is selected from the group consisting of apixaban, betrixaban, edoxaban, otamixaban, razaxaban, rivaroxaban, (r)-n-(2-(4-(1-methylpiperidin-4-yl) piperazin-1-yl)-2-oxo-1-phenylethyl)-lh-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl] acetic acid (YM-466 or YM-60828), eribaxaban (PD 0348292), and carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052).

18. The therapeutic composition of claim 17, wherein the direct factor Xa inhibitor comprises apixaban or a salt, isomer, solvate, analog, derivative, metabolite, or prodrug thereof.

19. The therapeutic composition of claim 17, wherein the direct factor Xa inhibitor comprises rivaroxaban or a salt, isomer, solvate, analog, derivative, metabolite, or prodrug thereof.

20. The therapeutic composition of claim 14, wherein the direct factor IIa inhibitor is selected from the group consisting of argatroban or a salt, isomer, solvate, derivative, analog, metabolite, or prodrug thereof, and wherein the direct factor Xa inhibitor comprises at least one of apixaban, rivaroxaban, or a salt, isomer, solvate, derivative, analog, metabolite, or prodrug of either apixaban or rivaroxaban.

21. The therapeutic composition of claim 2, wherein the mTOR inhibitor is selected from the group consisting of sirolimus, biolimus, everolimus, myolimus, novolimus, ridaforolimus, temsirolimus, zotarolimus, and salts, isomers, solvates, analogs, derivatives, metabolites, or prodrugs of any of the preceding.

22. The therapeutic composition of claim 21, wherein the mTOR inhibitor comprises sirolimus or a salt, isomer, solvate, analog, derivative, metabolite, or prodrug thereof.

23. The therapeutic composition of claim 2, wherein the therapeutic composition further comprises an antiproliferative agent selected from the group consisting of mycophenolate mofetil, mycophenolate sodium, and azathioprine.

24. The therapeutic composition of claim 2, wherein the antiplatelet drug comprises at least tirofiban or a salt, isomer, solvate, analog, derivative, metabolite, or prodrug thereof.

25. The therapeutic composition of claim 2, wherein therapeutic composition comprises additional active and/or inactive substances.

26. The therapeutic composition of claim 25, wherein the additional active and/or inactive substances are present in the therapeutic composition at a weight percent from 20% to 90%.

27. The therapeutic composition of claim 2, wherein the therapeutic composition comprises at least two of the following additional substances:
(a) a cationic anti-coagulation enhancer;
(b) an anti-coagulant;
(c) an mTOR inhibitor;
(d) paclitaxel or a salt, isomer, solvate, derivative, analog, metabolite, or prodrug thereof; or
(e) an antiplatelet drug.

28. The therapeutic composition of claim 2, wherein the therapeutic composition comprises at least three of the following additional substances:
(a) a cationic anti-coagulation enhancer;
(b) an anti-coagulant;
(c) an mTOR inhibitor;
(d) paclitaxel or a salt, isomer, solvate, derivative, analog, metabolite, or prodrug thereof; or
(e) an antiplatelet drug.

29. The therapeutic composition of claim 2, wherein the therapeutic composition comprises at least four of the following additional substances:
(a) a cationic anti-coagulation enhancer;
(b) an anti-coagulant;
(c) an mTOR inhibitor;
(d) paclitaxel or a salt, isomer, solvate, derivative, analog, metabolite, or prodrug thereof; or
(e) an antiplatelet drug.

30. The therapeutic composition of claim 2, wherein the therapeutic composition comprises all five of the following additional substances:
(a) a cationic anti-coagulation enhancer;
(b) an anti-coagulant;
(c) an mTOR inhibitor;
(d) paclitaxel or a salt, isomer, solvate, derivative, analog, metabolite, or prodrug thereof; or
(e) an antiplatelet drug.

31. An implantable scaffold comprising:
a scaffold structure having a surface configured to be expanded in a vascular environment in a patient's body; and
the therapeutic composition of claim 2 present on a surface of the scaffold structure.

32. The implantable scaffold of claim 31, wherein the therapeutic composition is formulated to release at least 50%, preferably at least 75%, by weight of the at least one chelating agent into a vascular environment within 72 hours of implantation, preferably within 24 hours of implantation, more preferably within 6 hours of implantation, and even more preferably within 4 hour of implantation.

33. The implantable scaffold of claim 32, wherein the therapeutic composition is formulated to release additional amounts of the at least one chelating agent into the vascular environment for a period of at least 3 days, preferably at least 7 days, more preferably 21 days, still more preferably at least 28 days, even more preferably at least 3 months, and often 6 months or more after implantation.

34. The implantable scaffold of claim 31, wherein the scaffold structure is configured to be expanded in a vascular lumen in the patient's body.

35. The implantable scaffold of claim 31, wherein the therapeutic composition is present at least partly on the surface of the scaffold structure.

36. The implantable scaffold of claim 31, wherein the therapeutic composition is present at least partly within a cavity or reservoir within the scaffold structure.
